SEQUENCE LISTING (80 sequences)

SEQ ID NO: 1 - RNA, 525 nt, Homo sapiens
gggagacaagcuggccgccaugccaagagaagaugcucacuucaucuaugguuaccccaagaaggggcacggccacucuuacaccacggcugaagaggccgcugggaucggcauccugacagugauccugggagucuuacugcucaucggcuguugguauuguagaagcgaaauggauacagagccuugauggauaaaagucuucauguuggcacucaaugugccuuaacaagaagaugcccacaagaaggguuugaucaucgggacagcaaagugucucuucaagagaaaaacugugaaccugugguucccaaugcuccaccugcuuaugagaaacucucugcagaacagucaccaccaccuuauucaccuuaagcggcccuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugcaggucgacucuag

SEQ ID NO: 2 - RNA, 2028 nt, Homo sapiens
gggagacaagcuaauuccugcuccuggcuguuuuguacugccugcuguggaguuuccagaccuccgcuggccauuucccuagagccugugucuccucuaagaaccugauggagaaggaaugcuguccaccguggagcgggacaggagucuguggccagcuuucaggcagagguuccugucagaauauccuucuguccaaugcaccacuugggccucaauuucccuucacagggguggaugaccgggagucguggccuuccgucuuuuauaauaggaccugccagugcucuggcaacuucaugggauucaacuguggaaacugcaaguuuggcuuuuggggaccaaacugcacagagagacgacucuuggugagaagaaacaucuucgauuugagugccccagagaaggacaaauuuuuugccuaccucacuuuagcaaagcauaccaucagcucagacuaugucauccccauagggaccuauggccaaaugaaaaauggaucaacacccauguuuaacgacaucaauauuuaugaccucuuugucuggaugcauuauuaugugucaauggaugcacugcuugggggauaugaaaucuggagagacauugauuuugcccaugaagcaccagcuuuucugccuuggcauagacucuucuuguugcggugggaacaagaaauccagaagcugacaggagaugaaaacuucacuauuccauauugggacuggcggggaugcagaaagugugacauuugcacagaugaguacaugggaggucagcaccccacaaaucuuaacuuacucagcccagcaucauucuucuccucuuggcagauugucuguagccgauuggaggaguacaacagccaucagucuuuaugcaauggaacgcccgagggaccuuuacggcguaaucuuggaaaccaugacaaauccacaaccccaaggcucccccucucagcugauguagaauuuugccugaguuugacccaauaugaaucugguuccauggauaaagcugccaauuucagcuuuagaaauacacuggaagaauuugcuaguccacuuacugggauagcggaugccucucaaagcagcaugcacaaugccuugcacaucuauaugaauggacaugcuccagguacaggacucgccaacgauccuaucuuccuucuucaccaugcauuuguugacaguauuuuugagcaguggcuccaaaggcaccguccucuucaagaaguuuauccagaagccaaugcacccauuggacauaaccgggaauccuacaugguucuuuuuauaccacuguacagaaauggugauuucuuuauuucauccaaagaucugggcuaugacuauagcuaucuacaagauucagacccagacucuuuucaagacuacauuaaguccuauuuggaacaagcgagucggaucuggucauggcuccuuggggcggcgaugguaggggccguccucacugcccugcuggcagggccugugagcuugcugugucgucacaagagaaagcagcuuccugaagaaaagcagccacuccucauggagaaagaaggauuaccacagcuuguaucagagccauuuauaaaaggcuuaggcaauagaguagggccaaaaagcccugaccucacucuaacucaaaguaauguccagguuccagagaauaucugcugguauuuucuguaaagaccauuugcaaaauuguaaccuaauacaaaguguagccuucuuccaacucagguagaacacaccugucuuugucuugcuguuuucacucagcccuuuuaacauuuuccccuaagcccauaugucuaaggaaaggaugcuauuugguaaugaggaacuguuauuuguaugugaauuaaaagugcucuuaggaauucuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugca

SEQ ID NO: 3 - RNA, 1086 nt, Homo sapiens
gggagacaagcuucaucaugucucuugagcagaggagucugcacugcaagccugaggaagcccuugaggcccaacaagaggcccugggccuggugugugugcaggcugccaccuccuccuccucuccucugguccugggcacccuggaggaggugcccacugcugggucaacagauccuccccagaguccucagggagccuccgccuuucccacuaccaucaacuucacucgacagaggcaacccagugaggguuccagcagccgugaagaggaggggccaagcaccucuuguauccuggaguccuuguuccgagcaguaaucacuaagaagguggcugauuugguugguuuucugcuccucaaauaucgagccagggagccagucacaaaggcagaaaugcuggagagugucaucaaaaauuacaagcacuguuuuccugagaucuucggcaaagccucugaguccuugcagcuggucuuuggcauugacgugaaggaagcagaccccaccggccacuccuauguccuugucaccugccuaggucucuccuaugauggccugcugggugauaaucagaucaugcccaagacaggcuuccugauaauuguccuggucaugauugcaauggagggcggccaugcuccugaggaggaaaucugggaggagcugagugugauggagguguaugaugggagggagcacagugccuauggggagcccaggaagcugcucacccaagauuuggugcaggaaaaguaccuggaguaccggcaggugccggacagugaucccgcacgcuaugaguuccugugggguccaagggcccucgcugaaaccagcuaugugaaaguccuugaguaugugaucaaggucagugcaagaguucgcuuuuucuucccaucccugcgugaagcagcuuugagagaggaggaagagggagucugagcaugaacuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugca

SEQ ID NO: 4 - RNA, 1098 nt, Homo sapiens
gggagacaagcuucaucaugccucuugagcagaggagucagcacugcaagccugaagaaggccuugaggcccgaggagaggcccugggccuggugggugcgcaggcuccugcuacugaggagcaggaggcugccuccuccucuucuacucuaguugaagucacccugggggaggugccugcugccgagucaccagauccuccccagaguccucagggagccuccagccuccccacuaccaugaacuacccucucuggagccaauccuaugaggacuccagcaaccaagaagaggaggggccaagcaccuucccugaccuggaguccgaguuccaagcagcacucaguaggaagguggccgaguugguucauuuucugcuccucaaguaucgagccagggagccggucacaaaggcagaaaugcuggggagugucgucggaaauuggcaguauuucuuuccugugaucuucagcaaagcuuccaguuccuugcagcuggucuuuggcaucgagcugauggaaguggaccccaucggccacuuguacaucuuugccaccugccugggccucuccuacgauggccugcugggugacaaucagaucaugcccaaggcaggccuccugauaaucguccuggccauaaucgcaagagagggcgacugugccccugaggagaaaaucugggaggagcugaguguguuagagguguuugaggggagggaagacaguaucuugggggaucccaagaagcugcucacccaacauuucgugcaggaaaacuaccuggaguaccggcagguccccggcagugauccugcauguuaugaauuccugugggguccaagggcccucguugaaaccagcuaugugaaaguccugcaccauaugguaaagaucaguggaggaccucacauuuccuacccaccccugcaugaguggguuuugagagagggggaagagugagucacuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugca

SEQ ID NO: 5 - RNA, 1098 nt, Homo sapiens; identical to SEQ ID NO: 4

SEQ ID NO: 6 - RNA, 3920 nt, Homo sapiens
gggagacaagcuuagcaccauggagcuggcggccuugugccgcugggggcuccuccucgcccucuugccccccggagccgcgagcacccaagugugcaccggcacagacaugaagcugcggcucccugccagucccgagacccaccuggacaugcuccgccaccucuaccagggcugccagguggugcagggaaaccuggaacucaccuaccugcccaccaaugccagccuguccuuccugcaggauauccaggaggugcagggcuacgugcucaucgcucacaaccaagugaggcaggucccacugcagaggcugcggauugugcgaggcacccagcucuuugaggacaacuaugcccuggccgugcuagacaauggagacccgcugaacaauaccaccccugucacaggggccuccccaggaggccugcgggagcugcagcuucgaagccucacagagaucuugaaaggaggggucuugauccagcggaacccccagcucugcuaccaggacacgauuuuguggaaggacaucuuccacaagaacaaccagcuggcucucacacugauagacaccaaccgcucucgggccugccaccccuguucuccgauguguaagggcucccgcugcuggggagagaguucugaggauugucagagccugacgcgcacugucugugccgguggcugugcccgcugcaaggggccacugcccacugacugcugccaugagcagugugcugccggcugcacgggccccaagcacucugacugccuggccugccuccacuucaaccacaguggcaucugugagcugcacugcccagcccuggucaccuacaacacagacacguuugaguccaugcccaaucccgagggccgguauacauucggcgccagcugugugacugccugucccuacaacuaccuuucuacggacgugggauccugcacccucgucugcccccugcacaaccaagaggugacagcagaggauggaacacagcggugugagaagugcagcaagcccugugcccgagugugcuauggucugggcauggagcacuugcgagaggugagggcaguuaccagugccaauauccaggaguuugcuggcugcaagaagaucuuugggagccuggcauuucugccggagagcuuugauggggacccagccuccaacacugccccgcuccagccagagcagcuccaaguguuugagacucuggaagagaucacagguuaccuauacaucucagcauggccggacagccugccugaccucagcgucuuccagaaccugcaaguaauccggggacgaauucugcacaauggcgccuacucgcugacccugcaagggcugggcaucagcuggcuggggcugcgcucacugagggaacugggcaguggacuggcccucauccaccauaacacccaccucugcuucgugcacacggugcccugggaccagcucuuucggaacccgcaccaagcucugcuccacacugccaaccggccagaggacgagugugugggcgagggccuggccugccaccagcugugcgcccgagggcacugcugggguccagggcccacccagugugucaacugcagccaguuccuucggggccaggagugcguggaggaaugccgaguacugcaggggcuccccagggaguaugugaaugccaggcacuguuugccgugccacccugagugucagccccagaauggcucagugaccuguuuuggaccggaggcugaccaguguguggccugugcccacuauaaggacccucccuucugcguggcccgcugccccagcggugugaaaccugaccucuccuacaugcccaucuggaaguuuccagaugaggagggcgcaugccagccuugccccaucaacugcacccacuccuguguggaccuggaugacaagggcugccccgccgagcagagagccagcccucugacguccaucgucucugcggugguuggcauucugcuggucguggucuuggggguggucuuugggauccucaucaagcgacggcagcagaagauccggaaguacacgaugcggagacugcugcaggaaacggagcugguggagccgcugacaccuagcggagcgaugcccaaccaggcgcagaugcggauccugaaagagacggagcugaggaaggugaaggugcuuggaucuggcgcuuuuggcacagucuacaagggcaucuggaucccugauggggagaaugugaaaauuccaguggccaucaaaguguugagggaaaacacaucccccaaagccaacaaagaaaucuuagacgaagcauacgugauggcuggugugggcuccccauaugucucccgccuucugggcaucugccugacauccacggugcagcuggugacacagcuuaugcccuauggcugccucuuagaccauguccgggaaaaccgcggacgccugggcucccaggaccugcugaacugguguaugcagauugccaaggggaugagcuaccuggaggaugugcggcucguacacagggacuuggccgcucggaacgugcuggucaagagucccaaccaugucaaaauuacagacuucgggcuggcucggcugcuggacauugacgagacagaguaccaugcagaugggggcaaggugcccaucaaguggauggcgcuggaguccauucuccgccggcgguucacccaccagagugauguguggaguuauggugugacugugugggagcugaugacuuuuggggccaaaccuuacgaugggaucccagcccgggagaucccugaccugcuggaaaagggggagcggcugccccagccccccaucugcaccauugaugucuacaugaucauggucaaauguuggaugauugacucugaaugucggccaagauuccgggaguuggugucugaauucucccgcauggccagggacccccagcgcuuuguggucauccagaaugaggacuugggcccagccagucccuuggacagcaccuucuaccgcucacugcuggaggacgaugacaugggggaccugguggaugcugaggaguaucugguaccccagcagggcuucuucuguccagacccugccccgggcgcugggggcaugguccaccacaggcaccgcagcucaucuaccaggaguggcgguggggaccugacacuagggcuggagcccucugaagaggaggcccccaggucuccacuggcacccuccgaaggggcuggcuccgauguauuugauggugaccugggaaugggggcagccaaggggcugcaaagccuccccacacaugaccccagcccucuacagcgguacagugaggaccccacaguaccccugcccucugagacugauggcuacguugccccccugaccugcagcccccagccugaauaugugaaccagccagauguucggccccagcccccuucgccccgagagggcccucugccugcugcccgaccugcuggugccacucuggaaagggccaagacucucuccccagggaagaauggggucgucaaagacguuuuugccuuugggggugccguggagaaccccgaguacuugacaccccagggaggagcugccccucagccccacccuccuccugccuucagcccagccuucgacaaccucuauuacugggaccaggacccaccagagcggggggcuccacccagcaccuucaaagggacaccuacggcagagaacccagaguaccugggucuggacgugccagugugaacuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugca

SEQ ID NO: 7 - RNA, 2263 nt, Homo sapiens
gggagacaagcuuauggagucucccucggccccuccccacagauggugcauccccuggcagaggcuccugcucacagccucacuucuaaccuucuggaacccgcccaccacugccaagcucacuauugaauccacgccguucaaugucgcagaggggaaggaggugcuucuacuuguccacaaucugccccagcaucuuuuuggcuacagcugguacaaaggugaaagaguggauggcaaccgucaaauuauaggauauguaauaggaacucaacaagcuaccccagggcccgcauacaguggucgagagauaauauaccccaaugcaucccugcugauccagaacaucauccagaaugacacaggauucuacacccuacacgucauaaagucagaucuugugaaugaagaagcaacuggccaguuccggguauacccggagcugcccaagcccuccaucuccagcaacaacuccaaacccguggaggacaaggaugcuguggccuucaccugugaaccugagacucaggacgcaaccuaccugugguggguaaacaaucagagccucccggucagucccaggcugcagcuguccaauggcaacaggacccucacucuauucaaugucacaagaaaugacacagcaagcuacaaaugugaaacccagaacccagugagugccaggcgcagugauucagucauccugaauguccucuauggcccggaugcccccaccauuuccccucuaaacacaucuuacagaucaggggaaaaucugaaccucuccugccaugcagccucuaacccaccugcacaguacucuugguuugucaaugggacuuuccagcaauccacccaagagcucuuuauccccaacaucacugugaauaauaguggauccuauacgugccaagcccauaacucagacacuggccucaauaggaccacagucacgacgaucacagucuaugcagagccacccaaacccuucaucaccagcaacaacuccaaccccguggaggaugaggaugcuguagccuuaaccugugaaccugagauucagaacacaaccuaccugugguggguaaauaaucagagccucccggucagucccaggcugcagcuguccaaugacaacaggacccucacucuacucagugucacaaggaaugauguaggacccuaugaguguggaauccagaacgaauuaaguguugaccacagcgacccagucauccugaauguccucuauggcccagacgaccccaccauuucccccucauacaccuauuaccguccaggggugaaccucagccucuccugccaugcagccucuaacccaccugcacaguauucuuggcugauugaugggaacauccagcaacacacacaagagcucuuuaucuccaacaucacugagaagaacagcggacucuauaccugccaggccaauaacucagccaguggccacagcaggacuacagucaagacaaucacagucucugcggagcugcccaagcccuccaucuccagcaacaacuccaaacccguggaggacaaggaugcuguggccuucaccugugaaccugaggcucagaacacaaccuaccugugguggguaaauggucagagccucccagucagucccaggcugcagcuguccaauggcaacaggacccucacucuauucaaugucacaagaaaugacgcaagagccuauguauguggaauccagaacucagugagugcaaaccgcagugacccagucacccuggauguccucuaugggccggacacccccaucauuucccccccagacucgucuuaccuuucgggagcgaaccucaaccucuccugccacucggccucuaacccauccccgcaguauucuuggcguaucaaugggauaccgcagcaacacacacaaguucucuuuaucgccaaaaucacgccaaauaauaacgggaccuaugccuguuuugucucuaacuuggcuacuggccgcaauaauuccauagucaagagcaucacagucucugcaucuggaacuucuccuggucucucagcuggggccacugucggcaucaugauuggagugcugguugggguugcucugauacuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugcaggucgacucuag

SEQ ID NO: 8 - RNA, 4246 nt, Homo sapiens
gggagacaagcuccgcuccaccucucaagcagccagcgccugccugaaucuguucugcccccuccccacccauuucaccaccaccaugacaccgggcacccagucuccuuucuuccugcugcugcuccucacagugcuuacaguuguuacagguucuggucaugcaagcucuaccccagguggagaaaaggagacuucggcuacccagagaaguucagugcccagcucuacugagaagaaugcugugaguaugaccagcagcguacucuccagccacagccccgguucaggcuccuccaccacucagggacaggaugucacucuggccccggccacggaaccagcuucagguucagcugccaccuggggacaggaugucaccucggucccagucaccaggccagcccugggcuccaccaccccgccagcccacgaugucaccucagccccggacaacaagccagccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccacggugucaccucggccccggacaccaggccggccccgggcuccaccgcccccccagcccauggugucaccucggccccggacaacaggcccgccuugggcuccaccgccccuccaguccacaaugucaccucggccucaggcucugcaucaggcucagcuucuacucuggugcacaacggcaccucugccagggcuaccacaaccccagccagcaagagcacuccauucucaauucccagccaccacucugauacuccuaccacccuugccagccauagcaccaagacugaugccaguagcacucaccauagcucgguaccuccucucaccuccuccaaucacagcacuucuccccaguugucuacuggggucucuuucuuuuuccugucuuuucacauuucaaaccuccaguuuaauuccucucuggaagaucccagcaccgacuacuaccaagagcugcagagagacauuucugaaauguuuuugcagauuuauaaacaaggggguuuucugggccucuccaauauuaaguucaggccaggaucuguggugguacaauugacucuggccuuccgagaagguaccaucaauguccacgacguggagacacaguucaaucaguauaaaacggaagcagccucucgauauaaccugacgaucucagacgucagcgugagugaugugccauuuccuuucucugcccagucuggggcuggggugccaggcuggggcaucgcgcugcuggugcuggucuguguucugguugcgcuggccauugucuaucucauugccuuggcugucugucagugccgccgaaagaacuacgggcagcuggacaucuuuccagcccgggauaccuaccauccuaugagcgaguaccccaccuaccacacccaugggcgcuaugugcccccuagcaguaccgaucguagccccuaugagaagguuucugcagguaacgguggcagcagccucucuuacacaaacccagcaguggcagccgcuucugccaacuuguagggcacgucgccgcugagcugaguggccagccagugccauuccacuccacucagguucuucaggccagagccccugcacccuguuugggcuggugagcugggaguucaggugggcugcucacagccuccuucagaggccccaccaauuucucggacacuucucaguguguggaagcucaugugggccccugaggcucaugccugggaaguguugugggggcucccaggaggacuggcccagagagcccugagauagcggggaucugugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugca

SEQ ID NO: 9 - RNA, 2173 nt, Homo sapiens
gggagacaagcuacacaauggaucuggugcuaaaaagaugccuucuucauuuggcugugauaggugcuuugcuggcugugggggcuacaaaaguacccagaaaccaggacuggcuuggugucucaaggcaacucagaaccaaagccuggaacaggcagcuguauccagaguggacagaagcccagagacuugacugcuggagagguggucaagugucccucaaggucaguaaugaugggccuacacugauuggugcaaaugccuccuucucuauugccuugaacuucccuggaagccaaaagguauugccagaugggcagguuaucugggucaacaauaccaucaucaaugggagccagguguggggaggacagccaguguauccccaggaaacugacgaugccugcaucuucccugaugguggaccuugcccaucuggcucuuggucucagaagagaagcuuuguuuaugucuggaagaccuggggccaauacuggcaaguucuagggggcccagugucugggcugagcauugggacaggcagggcaaugcugggcacacacaccauggaagugacugucuaccaucgccggggaucccggagcuaugugccucuugcucauuccagcucagccuucaccauuacugaccaggugccuuucuccgugagcgugucccaguugcgggccuuggauggagggaacaagcacuuccugagaaaucagccucugaccuuugcccuccagcuccaugacccuaguggcuaucuggcugaagcugaccucuccuacaccugggacuuuggagacaguaguggaacccugaucucucgggcaccuguggucacucauacuuaccuggagccuggcccagucacugcccaggugguccugcaggcugccauuccucucaccuccuguggcuccuccccaguuccaggcaccacagaugggcacaggccaacugcagaggccccuaacaccacagcuggccaagugccuacuacagaaguuguggguacuacaccuggucaggcgccaacugcagagcccucuggaaccacaucugugcaggugccaaccacugaagucauaagcacugcaccugugcagaugccaacugcagagagcacagguaugacaccugagaaggugccaguuucagaggucauggguaccacacuggcagagaugucaacuccagaggcuacagguaugacaccugcagagguaucaauuguggugcuuucuggaaccacagcugcacagguaacaacuacagaguggguggagaccacagcuagagagcuaccuaucccugagccugaagguccagaugccagcucaaucaugucuacggaaaguauuacagguucccugggcccccugcuggaugguacagccaccuuaaggcuggugaagagacaagucccccuggauuguguucuguaucgauaugguuccuuuuccgucacccuggacauuguccaggguauugaaagugccgagauccugcaggcugugccguccggugagggggaugcauuugagcugacuguguccugccaaggcgggcugcccaaggaagccugcauggagaucucaucgccagggugccagcccccugcccagcggcugugccagccugugcuacccagcccagccugccagcugguucugcaccagauacugaaggguggcucggggacauacugccucaaugugucucuggcugauaccaacagccuggcaguggucagcacccagcuuaucaugccugugccugggauucuucucacaggucaagaagcaggccuugggcagguucggcugaucgugggcaucuugcugguguugauggcugugguccuugcaucucugauauauaggcgcagacuuaugaagcaagacuucuccguaccccaguugccacauagcagcagucacuggcugcgucuaccccgcaucuucugcucuugucccauuggugagaauagcccccuccucagugggcagcaggucugaguacacuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugcaggucgacucuag

SEQ ID NO: 10 - RNA, 980 nt, Influenza virus
gggagacaagcuugcuuguucuuuuugcagaagcucagaauaaacgcucaacuuuggcagaucuggcagaucuaaagaugagucuucuaaccgaggucgaaacguacguucucucuaucgucccgucaggcccccucaaagccgagaucgcacagagacuugaagaugucuuugcugggaagaacacagaucuugaggcucucauggaauggcuaaagacaagaccaauccugucaccucugacuaaggggauuuugggauuuguauucacgcucaccgugcccagugagcgaggacugcagcguagacgcuuuguccaaaaugcccucaaugggaauggggauccaaauaacauggacagagcaguuaaacuguauagaaaacuuaagagggagauaacauuccauggggccaaagaaauagcacucaguuauucugcuggugcacuugccaguugcaugggccucauauacaacaggaugggggcugugaccacugaaguggccuuuggccugguaugugcaaccugugaacagauugcugacucccagcauaggucucauaggcaaauggugacaacaaccaaucccuaauaagacaugagaacagaaugguucuggccagcacuacagcuaaggcuauggagcaaauggcuggaucgagugagcaagcagcagaggccauggagguugcuagucaggcuaggcaaauggugcaagcgaugagaaccauugggacucauccuagcuccagugcuggucugaaaaaugaucuucuugaaaauuugcaggccuaucagaaacgaaugggggugcagaugcaacgguucaagugaacuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugcaggucgacucuag

SEQ ID NO: 11 - RNA, 972 nt, Artificial Sequence
  synthetic construct
gggagacaagcuugcuuguucuuuuugcagaagcucagaauaaacgcucaacuuuggcagaucuaaagaugagccugcugaccgagguggagaccuacgugcugagcaucauccccagcggcccccugaaggccgagaucgcccagaggcuggaggacguguucgccggcaagaacaccgaccuggaggugcugauggaguggcugaagaccaggcccauccugagcccccugaccaagggcauccugggcuucguguucacccugaccgugcccagcgagcgcggccugcagcgccgccgcuucgugcagaacgcccugaacggcaacggcgaccccaacaacauggacaaggccgugaagcuguacaggaagcugaagagggagaucaccuuccacggcgccaaggagaucagccugagcuacagcgccggcgcccuggccagcugcaugggccugaucuacaacaggaugggcgccgugaccaccgagguggccuucggccuggugugcgccaccugcgagcagaucgccgacagccagcaccgcagccacaggcagauggugaccaccaccaacccccugaucaggcacgagaacaggauggugcuggccagcaccaccgccaaggccauggagcagauggccggcagcagcgagcaggccgccgaggccauggagguggccagccaggccaggcagauggugcaggccaugaggaccaucggcacccaccccagcagcagcgccggccugaagaacgaccugcuggagaaccugcaggccuaccagaagcgcaugggcgugcagaugcagcgcuucaagugaacuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugcaggucgacucuag

SEQ ID NO: 12 - RNA, 1254 nt, Hepatitis B virus
gggagacgaauucccaaccuuccccaaacucugcaagaucccagagugagaggccuguauuucccugcugguggcuccaguucaggaacaguaaacccuguucugacuacugccucucccuuaucgucaaucuucucgaggauuggggacccugcgcugaacauggagaacaucacaucaggauuccuaggaccccuucucguguuacaggcgggguuuuucuuguugacaagaauccucacaauaccgcagagucuagacucgugguggacuucucucaauuuucuagggggaacuaccgugugucuuggccaaaauucgcaguccccaaccuccaaucacucaccaaccucuuguccuccaacuuguccugguuaucgcuggaugugucugcggcguuuuaucaucuuccucuucauccugcugcuaugccucaucuucuuguugguucuucuggacuaucaagguauguugcccguuuguccucuaauuccaggauccucaacaaccagcacgggaccaugccggaccugcaugacuacugcucaaggaaccucuauguaucccuccuguugcuguaccaaaccuucggacggaaauugcaccuguauucccaucccaucauccugggcuuucggaaaauuccuaugggagugggccucagcccguuucuccuggcucaguuuacuagugccauuuguucagugguucguagggcuuucccccacuguuuggcuuucaguuauauggaugaugugguauugggggccaagucuguacagcaucuugagucccuuuuuaccgcuguuaccaauuuucuuuugucuuuggguauacauuuaaacccuaacaaaacaaagagaugggguuacucucuaaauuuuauggguuaugucauuggauguuauggguccuugccacaagaacacaucauacaaaaaaucaaagaauguuuuagaaaacuuccuauuaacaggccuauugauuggaaaguaugucaacgaauugugggucuuuuggguuuugcugccccuuuuacacaaugugguuauccugcguugaugccuuuguacuagcuagugacugacuagcccgcugggccucccaacgggcccuccuccccuccuugcaccaaaaaaaaaaaaaaaaaaaaaaaaaaaaaaaaaaaaaaaaaaaaaaaaaaaaaaaaaaaaaaaaaaaaaacugcaggucgacucuagaggauccccggguaccgagcucgaauucguauuc

SEQ ID NO: 13 - RNA, 573 nt, Artificial Sequence
  synthetic construct
augaacuuccugcucagcugggugcacuggucgcuggcccugcugcuguaccugcaccacgcgaaguggagccaggccgccccgauggccgagggcgggggccagaaccaccacgaggucgugaaguucauggacguguaccagcggagcuacugccaccccaucgagacccucguggacaucuuccaggaguacccggacgagaucgaguacaucuucaagccgagcugcgugccgcugaugcgcugcggcggcugcugcaacgacgagggccuggagugcgugccgacggaggagagcaacaucaccaugcagaucaugcggaucaagccgcaccagggccagcacaucggggagaugagcuuccugcagcacaacaagugcgagugccggcccaagaaggaccgggcccggcaggagaacccgugcggcccgugcucggagcggcgcaagcaccuguucguccaggacccgcagaccugcaagugcagcugcaagaacaccgacagccggugcaaggcccggcagcuggagcucaacgagcggaccugccggugcgacaagccgcggcgc

SEQ ID NO: 14 - RNA, 4068 nt, Artificial Sequence
  synthetic construct
auggagagcaaggugcugcucgccgucgcgcuguggcugugcguggagacccgggccgccucggugggccugccgagcgugagccuggaccugccccgccucagcauccagaaggacauccugacgaucaaggccaacaccacccugcagaucaccugccgggggcagcgggaccuggacuggcuguggccgaacaaccagagcggcagcgagcagcggguggaggugaccgagugcucggacggccuguucugcaagacccucacgaucccgaaggucaucggcaacgacaccggcgccuacaagugcuucuaccgggagaccgaccuggccagcgugaucuacguguacgugcaggacuaccggagcccguucaucgcgagcgugagcgaccagcacggcguggucuacaucaccgagaacaagaacaagaccguggugaucccgugccuggggagcaucucgaaccugaacgugagccugugcgcccgcuacccggagaagcgguucgugcccgacggcaaccggaucagcugggacagcaagaagggcuucaccaucccgagcuacaugaucagcuacgccggcaugguguucugcgaggccaagaucaacgacgagucguaccagagcaucauguacaucgucgugguggugggcuaccggaucuacgacguggugcugagcccgagccacggcaucgagcucagcgucggggagaagcuggugcugaacugcacggcccggaccgagcugaacgugggcaucgacuucaacugggaguacccgagcucgaagcaccagcacaagaagcuggugaaccgggaccugaagacccagagcggcagcgagaugaagaaguuccucagcacccugaccaucgacggcgugacccgcagcgaccagggccuguacacgugcgccgcgagcucgggccugaugaccaagaagaacagcaccuucgugcggguccacgagaagccguucguggccuucgggagcggcauggagagccugguggaggccaccgugggcgagcgggugcggaucccggccaaguaccugggcuacccgccgcccgagaucaagugguacaagaacggcaucccgcucgagagcaaccacaccaucaaggccggccacgugcugaccaucauggaggucagcgagcgggacacggggaacuacaccgugauccugaccaacccgaucucgaaggagaagcagagccacguggugagccuggugguguacgucccgccccagaucggcgagaagagccugaucagcccgguggacagcuaccaguacggcaccacccagacccugacgugcaccguguacgccaucccgccgccgcaccacauccacugguacuggcagcucgaggaggagugcgcgaacgagccgucgcaggccgugagcgugaccaaccccuacccgugcgaggaguggcggagcguggaggacuuccagggcggcaacaagaucgaggucaacaagaaccaguucgcccugaucgagggcaagaacaagaccgugagcacccuggugauccaggccgccaacgugagcgcccuguacaagugcgaggcggugaacaagguggggcgcggcgagcgggucaucagcuuccacgugacccggggcccggagaucacgcugcagccggacaugcagccgaccgagcaggagucggugagccuguggugcaccgccgaccggagcaccuucgagaaccucaccugguacaagcugggcccgcagccccugccgauccacgugggcgagcugccgaccccggugugcaagaaccuggacacgcuguggaagcucaacgccaccauguucagcaacagcaccaacgacauccugaucauggagcugaagaacgccagccugcaggaccagggcgacuacgugugccuggcccaggaccggaagaccaagaagcggcacugcgucgugcgccagcugaccgugcucgagcggguggccccgaccaucacggggaaccuggagaaccagaccaccucgaucggcgagagcaucgaggugagcugcaccgcgagcggcaacccgcccccgcagaucaugugguucaaggacaacgagacccugguggaggacagcggcaucguccugaaggacggcaaccggaaccugaccauccggcgggugcggaaggaggacgagggccuguacacgugccaggccugcagcgugcucgggugcgccaagguggaggccuucuucaucaucgagggcgcccaggagaagaccaaccuggagaucaucauccuggugggcaccgccgugaucgcgauguucuucuggcugcugcuggucaucauccuccgcaccgugaagcgggccaacggcggcgagcugaagaccggcuaccugucgaucgugauggacccggacgagcugccgcuggacgagcacugcgagcggcugccguacgacgccagcaagugggaguucccgcgggaccggcucaagcuggggaagccccugggccggggcgccuucggccaggugaucgaggccgacgccuucggcaucgacaagaccgcgacgugccgcaccguggccgugaagaugcugaaggagggcgccacccacagcgagcaccgggcccugaugagcgagcugaagauccucauccacaucgggcaccaccugaacgucgugaaccugcugggcgccugcaccaagccgggcggcccgcugauggugaucguggaguucugcaaguucggcaaccugagcaccuaccuccggagcaagcggaacgaguucgugccguacaagaccaagggcgcccgguuccggcaggggaaggacuacgugggcgcgaucccggucgaccugaagcgccggcuggacucgaucacgagcagccagagcagcgccagcucgggcuucguggaggagaagagccugagcgacguggaggaggaggaggccccggaggaccuguacaaggacuuccugacccucgagcaccugaucugcuacagcuuccagguggccaagggcauggaguuccuggccagccggaagugcauccaccgggaccuggccgcgcggaacauccugcugagcgagaagaacguggugaagaucugcgacuucggccucgcccgggacaucuacaaggaccccgacuacguccgcaagggcgacgcccggcugccgcugaaguggauggccccggagaccaucuucgaccggguguacaccauccagucggacguguggagcuucggggugcugcugugggagaucuucagccugggcgccagcccguacccgggcgugaagaucgacgaggaguucugccggcggcucaaggagggcacccggaugcgcgccccggacuacaccacgcccgagauguaccagaccaugcuggacugcuggcacggcgagccgagccagcggccgaccuucagcgagcugguggagcaccugggcaaccugcugcaggcgaacgcccagcaggacgggaaggacuacaucguccucccgaucucggagacccugagcauggaggaggacagcggccugagccugccgaccagcccggugagcugcauggaggaggaggaggugugcgaccccaaguuccacuacgacaacaccgccggcaucucgcaguaccugcagaacagcaagcggaagagccggccggugagcgugaagacguucgaggacaucccgcuggaggagccggaggugaaggucaucccggacgacaaccagaccgacagcggcauggugcucgccagcgaggagcugaagacccuggaggaccggaccaagcugucgccgagcuucggcggcauggugcccagcaagagccgggagagcguggccagcgaggggucgaaccagaccagcggcuaccagagcggcuaccacagcgacgacaccgacacgaccguguacagcagcgaggaggccgagcugcugaagcucaucgagaucggcgugcagaccggcucgaccgcgcagauccugcagccggacagcggcaccacccugagcagcccgccgguc

SEQ ID NO: 15 - RNA, 1276 nt, Artificial Sequence
  synthetic construct
augcagggccggggcgaggaggccuuccgcgcgccgcacgggccccacggcgccgccggccugccgagccagccgucgugcuuccuccagaccggcgugcggagcgccaacaagggcccgcugauggucaucguggaguacugcaaguacgggaaccugagcaacuaccugaagagcaaguacgaccuguucuuccuggacaaggacguggccagccacauggagcggaagaaggagaagauggagccgggccucgagcagggcaagaagccgaagcuggacagcaucacgucgagcgagagcuucggcagcagcaaguuccaggaggacaagaaccugagcgacguggaggaggaggaggacucggacggcuucuaccaggagcccaucaccauggaggaccugaucagcuacagcuuccagguggcccggggcaugaaguuccugagcagccggaagugcauccacugggaccuggcggcccggaacauccuccugagcgagaacaacguggucaagaucugcgacuucgggcuggcccaggacaucuacaagaacgccgacuacgugcgcaagggcggcggcucgccguacccgggcgugcagauggacgagcacuucugcagcugccugcgggagggcaugcggaugcgggccgccgaguacagcaccccggagaucuaccagaucaugcuggacugccggcacaaggacccgaaggagcggccgcgcuucgcggagcugguggagaagcucggggaccugcugcaggccaacgugncaggacggcaaggacuacaucccccugagcgccauccugaccgagaacagcggcuucaccuacagcaccccggccuucucggaggacuucuucaaggagggcaucagcgccccgaaguucagcagcggcagcagcgacgacgugcgguacgucaacgccuucaaguucaugucgcuggagcggaucaagacguucgaggagcuccugccgaacgcgaccagcauguucgacgacuaccagggcgacagcagcgcccugcuggccagcccgaugcugaagcgguucacccggaccgacagcaagccgaaggccucgcugaagaucgaccuccggcugaccagcaagagcaagaagagcgggcugagcgacgugagccgccccucguucugccacagcaacagcggccacaucagcaagggcaagggccgguucaccuacgacaacgccgagcuggagcggaagacggccugcugcagcccgccgcugugggagcug

SEQ ID NO: 16 - RNA, 3318 nt, Artificial Sequence
  synthetic construct
augcggcugccgggcgccaugcccgcgcucgcccugaagggggagcugcugcugcugagccuccugcugcugcuggagccgcagaucucgcagggccugguggucaccccgccgggcccggagcucgugcugaacgugagcagcacguucgugcugaccugcagcggcagcgccccgguggugugggagcgcaugagccaggagcccccgcaggagauggccaaggcccaggacggcaccuucucgagcguccugacccugaccaaccugaccggccucgacacgggggaguacuucugcacccacaacgacagccggggccuggagaccgacgagcggaagcggcuguacaucuucgugccggacccgaccgugggcuuccugccgaacgacgccgaggagcuguucaucuuccugaccgagaucaccgagaucacgaucccgugccgggugaccgacccccagcucguggugacccugcacgagaagaagggcgacgucgcgcugccggugccguacgaccaccagcggggcuucuucggcaucuucgaggaccgcagcuacaucugcaagaccaccaucggggaccgggagguggacagcgacgccuacuacguguaccggcugcaggugagcucgaucaacgugagcgucaacgccgugcagaccguggugcggcagggcgagaacaucacgcugaugugcaucgugaucggcaacgagguggucaacuucgaguggaccuacccgcggaaggagagcggccggcugguggagccggugaccgacuuccuccuggacaugccguaccacauccgcagcauccugcacauccccagcgccgagcuggaggacagcggcaccuacaccugcaacgugaccgagucggugaacgaccaccaggacgagaaggccaucaacaucacgguggucgagagcggcuacgugcggcugcugggggaggugggcacccuccaguucgccgagcugcaccggagccggacccugcaggugguguucgaggcguacccgccgccgaccgugcugugguucaaggacaaccggacccugggcgacagcagcgccggcgagaucgcccugagcacccggaacgucucggagacgcgcuacgugagcgagcucacccuggugcgggugaagguggccgaggccggccacuacaccaugcgggccuuccacgaggacgcggaggugcagcugagcuuccagcugcagaucaacgucccggugcgggugcuggagcugagcgagagccacccggacagcggcgagcagaccgugcggugccgggggcgcggcaugccccagccgaacaucaucuggucggccugccgggaccucaagcggugcccgcgggagcugccgccgacccugcugggcaacagcagcgaggaggagagccagcuggagaccaacgugacguacugggaggaggagcaggaguucgagguggucagcacccugcggcuccagcacguggaccggccgcugagcgugcgcugcacccugcggaacgccgugggccaggacacccaggaggugaucguggucccccacucgcugccguucaaggugguggugaucagcgccauccuggcccugguggugcucaccaucaucagccugaucauccugaucaugcuguggcagaagaagccgcgguacgagauccgguggaaggucaucgagagcgugagcagcgacggccacgaguacaucuacguggacccgaugcagcugccguacgacucgaccugggagcugccgcgggaccagcucgugcugggccggacgcuggggagcggcgccuucggccaggugguggaggcgaccgcccacggccugagccacagccaggccaccaugaaggucgccgugaagaugcugaagagcaccgcccgcagcucggagaagcaggcccugaugagcgagcucaagaucaugagccaccugggcccccaccugaacguggugaaccugcugggcgcgugcaccaagggcgggccgaucuacaucaucaccgaguacugccgguacggcgaccugguggacuaccuccaccggaacaagcacacguuccugcagcaccacagcgacaagcggcggccgccgagcgccgagcuguacagcaacgcccugccggugggccugccgcugcccucgcacgucagccucaccggcgagagcgacggcggguacauggacaugagcaaggacgagagcguggacuacgugccgaugcuggacaugaagggcgacgugaaguacgccgacaucgagagcucgaacuacauggccccguacgacaacuacgugccgagcgccccggagcggaccugccgcgcgacccugaucaacgagagcccggugcugagcuacauggaccuggucggcuucagcuaccagguggccaacggcauggaguuccuggccagcaagaacugcgugcaccgggaccucgccgcccggaacgugcugaucugcgagggcaagcuggugaagaucugcgacuucggccuggcccgggacaucaugcgggacucgaacuacaucagcaaggggagcaccuuccugccccugaaguggauggcgccggagagcaucuucaacagccucuacaccacgcugagcgacguguggucguucggcauccugcugugggagaucuucacccugggcggcaccccguacccggagcugccgaugaacgagcaguucuacaacgccaucaagcggggcuaccgcauggcccagccggcccacgccagcgacgagaucuacgagaucaugcagaagugcugggaggagaaguucgagauccggcccccguucagccagcucguccugcugcuggagcggcugcugggcgagggguacaagaagaaguaccagcagguggacgaggaguuccuccggagcgaccacccggccauccugcggagccaggcgcggcugccgggcuuccacggccugcgcagcccgcuggacaccucgagcgugcuguacaccgccgugcagccgaacgagggcgacaacgacuacaucaucccccucccggacccgaagccggagguggccgacgagggcccgcuggagggcagcccgagccuggccagcagcacccugaacgaggugaacacgucgagcaccaucagcugcgacagcccccuggagccgcaggacgagccggagccggagccgcagcuggagcuccaggucgagccggagcccgagcuggagcagcugccggacagcgggugcccggccccgcgggccgaggcggaggacagcuuccug

SEQ ID NO: 17 - RNA, 4026 nt, Artificial Sequence
  synthetic construct
augcgggccaacgacgcgcugcaggugcucggccugcuguucagccuggcccgcgggucggaggucggcaacagccaggccgugugcccgggcacccugaacggccugagcgugacgggcgacgccgagaaccaguaccagacccucuacaagcuguacgagcggugcgagguggugaugggcaaccuggagaucgugcugaccgggcacaacgccgaccugagcuuccugcaguggauccgggaggucaccggcuacgugcucguggccaugaacgaguucagcacccugccccugccgaaccugcggguggugcggggcacccagguguacgacggcaaguucgcgaucuucgucaugcugaacuacaacacgaacagcucgcacgcccugcggcagcuccgccugacccagcugaccgagauccugagcggcggcguguacaucgagaagaacgacaagcugugccacauggacaccaucgacuggcgggacaucgugcgggaccgggacgccgagaucguggugaaggacaacgggcggagcugcccgccgugccacgaggugugcaagggccggugcuggggcccgggcagcgaggacugccagacccugaccaagacgaucugcgccccgcagugcaacggccacugcuucggccccaacccgaaccagugcugccacgacgagugcgccgggggcugcagcggcccgcaggacaccgacugcuucgccugccgccacuucaacgacagcggcgcgugcgucccgcggugcccgcagccgcucguguacaacaagcugaccuuccagcuggagcccaacccgcacaccaaguaccaguacggcggcgugugcguggccucgugcccgcacaacuucgugguggaccagaccagcugcguccgggccugcccgccggacaagauggagguggacaagaacgggcugaagaugugcgagccgugcggcggccugugccccaaggccugcgagggcaccggcagcggcagccgguuccagacgguggacagcagcaacaucgacggguucgugaacugcaccaagauccugggcaaccucgacuuccugaucaccggccugaacggcgacccguggcacaagaucccggcccuggacccggagaagcugaacguguuccggaccgugcgggagaucaccggcuaccugaacauccagucguggccgccgcacaugcacaacuucagcgucuucagcaaccucaccacgaucggcgggcgcagccuguacaaccggggcuucagccugcugaucaugaagaaccugaacgugaccagccugggcuuccggucgcucaaggagaucagcgccggccggaucuacaucagcgcgaaccggcagcugugcuaccaccacagccugaacuggaccaaggugcugcggggccccaccgaggagcgccuggacaucaagcacaaccggccgcggcgggacugcguggccgagggcaaggugugcgacccgcugugcagcagcgggggcugcuggggcccgggcccgggccagugccucucgugccggaacuacagccggggcggggugugcgucacccacugcaacuuccugaacggcgagccgcgcgaguucgcccacgaggccgagugcuucagcugccaccccgagugccagccgaugggcggcaccgccacgugcaacggcagcggcagcgacaccugcgcccagugcgcgcacuuccgggacgggccgcacugcgugagcucgugcccgcacggcgugcugggcgccaagggcccgaucuacaaguacccggacgugcagaacgagugccggcccugccacgagaacugcacccagggcugcaagggcccggagcugcaggacugccuggggcagacccuggugcucaucggcaagacccaccugaccauggcccugacggugaucgccggccuggucgugaucuucaugaugcugggcggcaccuuccuguacuggcggggccggcggauccagaacaagcgcgccaugcggcgguaccucgagcggggcgagagcaucgagccgcuggacccgagcgagaaggccaacaaggugcuggcgcggaucuucaaggagaccgagcugcggaagcugaaggugcuggggagcggcguguucggcaccgugcacaagggcgucuggaucccggagggcgagagcaucaagaucccggugugcaucaaggugaucgaggacaagagcgggcgccagucguuccaggccgugaccgaccacaugcucgccaucggcagccuggaccacgcccacaucgugcggcugcugggccugugccccggcagcagccugcagcucgugacccaguaccugccgcugggcagccugcuggaccacguccggcagcaccggggcgcccuggggccgcagcuccugcugaacuggggcgugcagaucgccaagggcauguacuaccuggaggagcacggcauggugcaccggaaccuggcggcccggaacgugcugcucaagagcccgucgcaggugcagguggccgacuucggcgucgccgaccugcugccgccggacgacaagcagcugcuguacagcgaggccaagacgcccaucaaguggauggcccuggagagcauccacuucggcaaguacacccaccagagcgacguguggagcuacggggugaccgugugggagcucaugaccuucggcgcggagccguacgccggccugcgccuggccgaggugccggaccugcuggagaagggcgagcggcuggcccagccgcagaucugcaccaucgacguguacauggucauggugaagugcuggaugaucgacgagaacauccggccgaccuucaaggagcucgccaacgaguucacgcggauggcccgggacccgccccgguaccuggugaucaagcgcgagagcggcccgggcaucgcgccggggccggagccgcacggccugaccaacaagaagcuggaggagguggagcuggagccggagcuggaccucgaccuggaccuggaggccgaggaggacaaccuggccaccaccacccugggcucggcccugagccuccccgugggcacccugaaccggccgcggggcagccagagccugcugagcccgagcucgggcuacaugccgaugaaccaggggaaccugggcggcagcugccaggagagcgccgugagcggcagcagcgagcggugcccgcggccggucucgcugcaccccaugccgcggggcugccucgccagcgagagcagcgagggccacgugacggggagcgaggcggagcugcaggagaaggugagcaugugccgcucgcggagccggagccggagcccgcggccgcggggcgacagcgccuaccacagccagcgccacucgcugcugaccccggugaccccgcugagcccgcccggccuggaggaggaggacgugaacggcuacgugaugccggacacccaccucaagggcaccccgagcagccgggagggcacccugagcagcgucgggcugucgagcgugcugggcacggaggaggaggacgaggacgaggaguacgaguacaugaaccggcggcggcggcacagcccgccgcacccgccgcgccccagcagccuggaggagcugggcuacgaguacauggacgugggcagcgaccucucggccagccugggcagcacccagagcugcccgcugcacccggugccgaucaugcccaccgccggcaccaccccggacgaggacuacgaguacaugaaccggcagcgggacgggggcggcccgggcggcgacuacgccgccaugggcgcgugcccggccagcgagcagggguacgaggagaugcgggccuuccagggcccgggccaccaggccccgcacgugcacuacgcccggcugaagacccugcggagccuggaggccacggacucggcguucgacaaccccgacuacuggcacagccgccucuucccgaaggccaacgcccagcggacc

SEQ ID NO: 18 - RNA, 942 nt, Artificial Sequence
  synthetic construct
auggccccgccccaggugcuggcguucggccuccugcuggccgccgccaccgccacguucgccgcggcccaggaggagugcgucugcgagaacuacaagcuggccgugaacugcuucgugaacaacaaccggcagugccagugcaccagcgugggggcccagaacaccgugaucugcucgaagcuggccgccaagugccuggugaugaaggcggagaugaacggcagcaagcucggccgccgggccaagccggagggcgcccugcagaacaacgacggccuguacgacccggacugcgacgagagcggccuguucaaggccaagcagugcaacgggaccagcaccugcuggugcgucaacaccgccggcgugcggcggacggacaaggacaccgagaucaccugcagcgagcgggugcggaccuacuggaucaucaucgagcugaagcacaaggcccgcgagaagccguacgacagcaagucgcugcggaccgcgcuccagaaggagaucaccacgcgguaccagcuggacccgaaguucaucaccagcauccuguacgagaacaacgugaucaccaucgaccuggugcagaacagcagccagaagacccagaacgacguggacaucgccgacgucgccuacuacuucgagaaggacgugaagggcgagagccuguuccacagcaagaagauggaccugaccgugaacggcgagcagcucgaccuggacccgggccagacccugaucuacuacguggacgagaaggcccccgaguucucgaugcagggccugaaggccggggugaucgccgugaucgucgugguggugaucgcggugguggccggcaucgucgugcuggugaucagccggaagaagcggauggccaaguacgagaaggccgagaucaaggagaugggcgagaugcaccgggagcugaacgcc

SEQ ID NO: 19 - RNA, 2250 nt, Artificial Sequence
  synthetic construct
auguggaaccugcuccacgagaccgacagcgccguggcgacggcccggcgcccgcgguggcugugcgccggcgcccugguccuggccgggggcuucuuccugcugggcuuccucuucggcugguucaucaagucgagcaacgaggccaccaacaucacccccaagcacaacaugaaggcguuccuggacgagcugaaggccgagaacaucaagaaguuccugcacaacuucacccagaucccgcaccuggccggcaccgagcagaacuuccagcuggccaagcagauccagagccaguggaaggaguucggccucgacagcguggagcuggcccacuacgacgugcugcugagcuacccgaacaagacccacccgaacuacaucagcaucaucaacgaggacgggaacgagaucuucaacacgucgcuguucgagccgccgcccccgggcuacgagaacgugagcgacaucgugccgccguucagcgccuucagcccgcagggcaugccggagggcgaccugguguacgucaacuacgcgcggaccgaggacuucuucaagcucgagcgggacaugaagaucaacugcagcggcaagaucgugaucgcccgguacggcaagguguuccgggggaacaaggugaagaacgcccagcuggccggcgccaagggcgugauccuguacagcgaccccgccgacuacuucgcgccgggcgugaagucguacccggacggcuggaaccugccgggcgggggcguccagcgcggcaacauccugaaccugaacggcgccggcgacccgcucaccccgggcuaccccgccaacgaguacgccuaccggcgggggaucgccgaggccgugggccugccgagcaucccggugcacccgaucggcuacuacgacgcgcagaagcugcuggagaagaugggcggcagcgccccgccggacagcagcuggcggggcagccugaaggugcccuacaacguggggccgggcuucaccggcaacuucucgacccagaaggugaagaugcacauccacagcaccaacgaggucacgcggaucuacaacgugaucggcacccugcggggcgccguggagccggaccgcuacgugauccucggcgggcaccgggacagcuggguguucggcggcaucgacccgcagagcggcgccgccgugguccacgagaucgugcggagcuucggcacccugaagaaggagggcuggcggccgcggcggaccauccuguucgccagcugggacgcggaggaguucgggcugcugggcucgaccgagugggccgaggagaacagccgccugcuccaggagcggggcguggccuacaucaacgccgacagcagcaucgagggcaacuacacccugcggguggacugcacgccgcugauguacagccuggugcacaaccugaccaaggagcugaagagccccgacgagggcuucgagggcaagucgcucuacgagagcuggaccaagaagagcccgagcccggaguucagcgggaugccgcggaucagcaagcugggcucgggcaacgacuucgagguguucuuccagcggcugggcaucgccagcggccgggcccgcuacaccaagaacugggagaccaacaaguucagcggcuacccgcuguaccacagcgucuacgagaccuacgagcugguggagaaguucuacgacccgauguucaaguaccaccugacgguggcgcaggugcggggcgggaugguguucgagcucgccaacagcaucgugcugcccuucgacugccgggacuacgccgucgugcugcggaaguacgccgacaagaucuacagcaucucgaugaagcacccgcaggagaugaagaccuacagcgugagcuucgacagccuguucagcgccgugaagaacuucaccgagaucgccagcaaguucucggagcggcugcaggacuucgacaagagcaacccgaucgugcugcggaugaugaacgaccagcucauguuccuggagcgcgcguucaucgacccgcugggccugccggaccggccguucuaccggcacgugaucuacgcccccagcagccacaacaaguacgccggcgagagcuucccgggcaucuacgacgcccuguucgacaucgagagcaaggucgacccgucgaaggccuggggcgaggugaagcggcagaucuacguggccgcguucaccgugcaggccgccgccgagacccugagcgagguggcc

SEQ ID NO: 20 - RNA, 783 nt, Artificial Sequence
  synthetic construct
augugggugccggucguguuccugacccucagcgugacguggaucggcgccgcgccccugauccugucgcggaucgugggcgggugggagugcgagaagcacagccagccguggcaggugcugguggccagccgcggccgggccgucugcggcggcgugcuggugcacccgcagugggugcugaccgccgcccacugcauccggaacaagagcgugauccuccugggccggcacagccuguuccacccggaggacaccgggcagguguuccaggucagccacucguucccgcacccgcuguacgacaugagccugcugaagaaccgguuccuccggcccggcgacgacagcagccacgaccugaugcugcugcgccugagcgagccggccgagcugaccgacgcggugaaggugauggaccucccgacccaggagccggcccugggcaccacgugcuacgccagcggcuggggcucgaucgagccggaggaguuccugaccccgaagaagcugcagugcguggaccugcacgugaucagcaacgacgugugcgcccagguccacccccagaaggugaccaaguucaugcugugcgccggccgguggaccgggggcaagagcaccugcagcggcgacagcggcggcccgcucgugugcaacggcgugcugcaggggaucaccagcuggggcucggagccgugcgcccugccggagcggccgagccuguacacgaagguggugcacuaccggaaguggaucaaggacaccaucgucgcgaacccg

SEQ ID NO: 21 - RNA, 369 nt, Artificial Sequence
  synthetic construct
augaaggccgugcugcucgcgcugcugauggccggccuggcccugcagccggggaccgcccugcucugcuacagcugcaaggcccaggucucgaacgaggacugccugcagguggagaacugcacgcagcugggcgagcagugcuggaccgcccggauccgcgcggugggccugcugaccgugaucagcaagggcugcagccugaacugcguggacgacagccaggacuacuacgugggcaagaagaacaucaccugcugcgacaccgaccucugcaacgccagcggcgcccacgcccugcagcccgccgccgcgauccuggcccugcugccggcccuggggcuccugcuguggggcccgggccagcug

SEQ ID NO: 22 - RNA, 1398 nt, Artificial Sequence
  synthetic construct
augagcacccggucggugagcagcagcagcuaccgccggauguucggcgggccgggcacggccagccggcccucgagcagccggagcuacgucaccaccagcacccggaccuacagccugggcucggcgcuccggccgagcaccagccgcagccuguacgccagcagcccgggcggcguguacgccacgcggucgagcgccgugcggcugcggagcagcgugccgggcgugcggcugcugcaggacagcguggacuucagccuggccgacgccaucaacaccgaguucaagaacacccggaccaacgagaaggucgagcuccaggagcugaacgaccgcuucgcgaacuacaucgacaaggugcgguuccuggagcagcagaacaagauccugcuggccgagcuggagcagcucaaggggcagggcaagucgcggcugggcgaccuguacgaggaggagaugcgggagcugcggcggcagguggaccagcugaccaacgacaaggcccgcguggagguggagcgggacaaccuggccgaggacaucaugcggcuccgggagaagcugcaggaggagaugcugcagcgggaggaggccgagaacacccugcagagcuuccggcaggacguggacaacgccagccuggcgcgccuggaccucgagcggaaggucgagagccugcaggaggagaucgccuuccugaagaagcugcacgaggaggagauccaggagcugcaggcccagauccaggagcagcacgugcagaucgacguggacgugagcaagccggaccugacggccgcccuccgggacgugcggcagcaguacgagagcguggccgcgaagaaccugcaggaggccgaggagugguacaagucgaaguucgccgaccugagcgaggccgccaaccggaacaacgacgcccugcggcaggcgaagcaggagagcaccgaguaccgccggcagguccagagccugaccugcgagguggacgcccugaagggcaccaacgagagccucgagcggcagaugcgggagauggaggagaacuucgccguggaggccgccaacuaccaggacaccaucggccggcugcaggacgagauccagaacaugaaggaggagauggcccggcaccugcgcgaguaccaggaccugcugaacgugaagauggcgcuggacaucgagaucgccaccuaccggaagcuccuggagggcgaggagagccggaucucgcugccgcugcccaacuucagcagccugaaccugcgggagacgaaccucgacagccugccgcugguggacacccacagcaagcggaccuuccugaucaagaccguggagacccgggacgggcaggucaucaacgagaccagccagcaccacgacgaccuggag

SEQ ID NO: 23 - RNA, 1311 nt, Artificial Sequence
  synthetic construct
auggccagcguggcggucgacccgcagcccucgguggugacccggguggugaaccugccgcucgugagcagcacguacgaccugaugagcagcgccuaccugagcaccaaggaccaguacccguaccugaagucggucugcgagauggccgagaacggcgugaagaccaucaccagcguggccaugaccagcgcccugccgaucauccagaagcuggagccgcagaucgccguggcgaacaccuacgccugcaaggggcucgaccgcaucgaggagcggcugccgauccugaaccagcccagcacgcagaucguggccaacgccaagggcgccgugaccggcgccaaggacgcggucaccaccaccgugaccggcgccaaggacagcguggccagcacgaucaccggcgugauggacaagaccaagggcgccgugaccgggucgguggagaagaccaagagcgucgugagcggcagcaucaacaccgugcugggcagccggaugaugcagcuggugagcucgggcguggagaacgcccugacgaagagcgagcuccugguggagcaguaccugccgcugaccgaggaggagcuggagaaggaggccaagaaggucgagggcuucgaccuggugcagaagccgagcuacuacgugcggcucggcagccugagcaccaagcugcacagccgggcguaccagcaggcccugucgcgggugaaggaggccaagcagaagagccagcagaccaucagccagcugcacagcaccgugcaccugaucgaguucgcccgcaagaacguguacagcgccaaccagaagauccaggacgcccaggacaagcucuaccugagcugggucgaguggaagcggucgaucggguacgacgacaccgacgagagccacugcgcggagcaguucgagagccggacgcuggccaucgcccggaaccugacccagcagcugcagaccaccugccacacccugcucagcaacauccagggcgugccgcagaacauccaggaccaggccaagcacaugggcgugauggccggcgacaucuacagcguguuccggaacgccgcgagcuucaaggaggugucggacagccugcugaccagcagcaagggccagcugcagaagaugaaggagagccuggacgacgugauggacuaccuggucaacaacacgccgcucaacuggcuggugggcccguucuacccccagcugaccgagagccagaacgcccaggaccagggggccgagauggacaagucgagccaggagacccagcggagcgagcacaagacccac

SEQ ID NO: 24 - RNA, 1125 nt, Artificial Sequence
  synthetic construct
auggacgacgacaucgccgcgcugguggucgacaacggcagcgggaugugcaaggccggcuucgccggcgacgacgccccgcgggccguguuccccucgaucgugggccgcccgcggcaccagggcgugauggugggcauggggcagaaggacagcuacgugggcgacgaggcccagagcaagcggggcauccucacccugaaguacccgaucgagcacggcaucgucacgaacugggacgacauggagaagaucuggcaccacaccuucuacaacgagcugcggguggcgccggaggagcacccggugcugcugaccgaggccccgcugaaccccaaggccaaccgggagaagaugacccagaucauguucgagaccuucaacaccccggccauguacguggccauccaggccgugcucagccuguacgcgagcggccggacgaccggcaucgugauggacagcggggacggcgucacccacaccgugccgaucuacgagggcuacgcccugccgcacgccauccugcgccuggaccuggccggccgggaccucaccgacuaccugaugaagauccugaccgagcggggcuacucguucacgaccaccgccgagcgggagaucgugcgggacaucaaggagaagcugugcuacguggcccuggacuucgagcaggagauggcgaccgccgccagcagcagcagccuggagaagagcuacgagcucccggacggccaggugaucaccaucgggaacgagcgguuccgcugcccggaggcccuguuccagcccucguuccugggcauggagagcugcggcauccacgagaccacguucaacagcaucaugaagugcgacguggacauccggaaggaccuguacgccaacaccguccugagcggcggcaccaccauguacccgggcaucgccgaccggaugcagaaggagaucaccgcgcuggccccgagcaccaugaagaucaagaucaucgccccgccggagcggaaguacagcguguggaucgggggcucgauccucgccagccugagcacguuccagcagauguggaucagcaagcaggaguacgacgagagcggcccgagcaucgugcaccggaagugcuuc

SEQ ID NO: 25 - RNA, 4224 nt, Artificial Sequence
  synthetic construct
augaaggccccggcggugcuggcccccggcauccucguccugcuguucacccuggugcagcggagcaacggggagugcaaggaggcccuggccaagucggagaugaacgugaacaugaaguaccagcugccgaacuucacggccgagaccccgauccagaacgugauccuccacgagcaccacaucuuccugggcgccaccaacuacaucuacgugcugaacgaggaggaccugcagaagguggcggaguacaagaccggcccgguccuggagcacccggacugcuucccgugccaggacugcagcagcaaggccaaccugagcggcggcguguggaaggacaacaucaacauggcccucgugguggacaccuacuacgacgaccagcugaucagcugcggcagcgugaaccgcgggaccugccagcggcacguguucccccacaaccacacggccgacauccagucggagguccacugcaucuucagcccgcagaucgaggagccgagccagugcccggacugcguggugagcgcccugggcgccaaggugcugagcagcgugaaggaccgguucaucaacuucuucgugggcaacaccaucaacucgagcuacuucccggaccacccgcugcacagcaucagcguccggcggcugaaggagaccaaggacggcuucauguuccucaccgaccagagcuacaucgacgugcugcccgaguuccgggacagcuacccgaucaaguacgugcacgcguucgagucgaacaacuucaucuacuuccugaccgugcagcgcgagacccuggacgcccagacguuccacacccggaucauccgguucugcagcaucaacagcggccugcacagcuacauggagaugccgcuggagugcauccucaccgagaagcggaagaagcggagcaccaagaaggagguguucaacauccugcaggccgccuacgugagcaagccgggcgcccagcuggcccggcagaucggggcgucgcugaacgacgacauccuguucggcgucuucgcccagagcaagccggacagcgccgagccgauggaccgcagcgccaugugcgccuuccccaucaaguacgugaacgacuucuucaacaagaucgugaacaagaacaacgugcggugccugcagcacuucuacggcccgaaccacgagcacugcuucaaccggacccuccugcggaacagcagcggcugcgaggcccggcgggacgaguaccgcaccgaguucacgaccgcgcugcagcggguggaccuguucaugggccaguucucggaggugcugcugaccagcaucagcaccuucaucaagggcgaccucaccaucgccaaccuggggaccagcgagggccgguucaugcaggucguggugagccggagcggcccgucgacgccgcacgugaacuuccugcuggacagccacccggugagcccggaggugaucgucgagcacacccugaaccagaacggcuacacccuggugaucaccggcaagaagaucaccaagaucccccucaacggccuggggugccggcacuuccagagcugcagccagugccugagcgccccgccguucgugcagugcggcuggugccacgacaagugcgugcggucggaggagugccugagcggcaccuggacgcagcagaucugccugccggccaucuacaagguguucccgaacagcgccccgcuggagggcggcacccgccucaccaucugcggcugggacuucggguuccggcggaacaacaaguucgaccugaagaagacccgggugcugcugggcaacgagagcugcacccugacccugagcgagagcacgaugaacacccucaagugcaccgucggccccgccaugaacaagcacuucaacaugucgaucaucaucagcaacggccacggcaccacccaguacagcaccuucagcuacguggacccggugaucacgagcaucagcccgaaguacggcccgauggcgggcgggacccugcugacccugaccggcaacuaccugaacucgggcaacagccggcacaucagcaucggcggcaagaccugcacccugaagagcgugagcaacagcauccucgagugcuacacgccggcccagaccaucucgaccgaguucgccgugaagcugaagaucgaccuggccaaccgggagaccagcaucuucagcuaccgcgaggacccgaucguguacgagauccaccccaccaagagcuucaucagcaccugguggaaggagccgcugaacaucgucagcuuccuguucugcuucgccucgggcgggagcacgaucaccggcgugggcaagaaccugaacagcgugagcgugccgcggauggugaucaacgugcacgaggccggccggaacuucaccgucgcgugccagcaccggagcaacagcgagaucaucugcugcaccaccccgucgcuccagcagcugaaccugcagcugccgcugaagaccaaggccuucuucaugcuggacggcauccucagcaaguacuucgaccugaucuacgugcacaacccgguguucaagcccuucgagaagccggugaugaucagcauggggaacgagaacgugcuggagaucaagggcaacgacaucgacccggaggccgugaagggcgagguccugaaggugggcaacaagagcugcgagaacauccaccugcacagcgaggccgugcugugcacggugccgaacgaccuccugaagcugaacagcgagcugaacaucgaguggaagcaggccaucucgagcaccgugcugggcaaggugaucguccagccggaccagaacuucaccggccugaucgccgggguggugagcaucagcaccgcgcuccugcugcugcugggcuucuuccuguggcucaagaagcggaagcagaucaaggaccugggcagcgagcuggugcgguacgacgcccgcgugcacaccccgcaccuggaccggcuggugagcgcccggucggucagccccaccacggagauggugagcaacgagagcguggacuaccgggccaccuucccggaggaccaguucccgaacagcagccagaacggcucgugccggcaggugcaguacccgcugaccgacaugagcccgauccucaccagcggcgacagcgacaucagcagcccgcugcugcagaacaccgugcacaucgaccugucggcccugaaccccgagcuggugcaggccguccagcacguggugaucggcccgagcagccucaucgugcacuucaacgaggugaucgggcggggccacuucggcugcguguaccacggcacccugcuggacaacgacggcaagaagauccacugcgcggucaagagccugaaccgcaucacggacaucggcgaggugagccaguuccugaccgaggggaucaucaugaaggacuucagccacccgaacgugcugucgcuccugggcaucugccugcggagcgagggcagcccgcugguggugcugccguacaugaagcacggcgaccugcggaacuucauccggaacgagacccacaacccgaccgugaaggaccucaucggcuucggccugcaggucgccaaggccaugaaguaccuggccagcaagaaguucgugcaccgggaccuggccgcccggaacugcaugcuggacgagaaguucaccgugaagguggcggacuucgggcuggcccgcgacauguacgacaaggaguacuacagcgugcacaacaagaccggcgccaagcuccccgugaaguggauggcccuggagagccugcagacgcagaaguucaccaccaagucggacgucuggagcuucggcguggugcugugggagcugaugacccggggcgccccgccguacccggacgugaacaccuucgacaucaccguguaccugcuccagggccggcggcugcugcagccggaguacugcccggacccccuguacgaggugaugcugaagugcuggcacccgaaggccgagaugcggccgagcuucagcgagcuggucagccggaucagcgcgaucuucucgacguucaucggcgagcacuacgugcacgugaacgccaccuacgugaacgugaagugcguggccccguacccgagccuccugagcagcgaggacaacgccgacgacgaggucgacacccgcccggccagcuucugggagaccagc

SEQ ID NO: 26 - RNA, 1377 nt, Artificial Sequence
  synthetic construct
auggccccgcugugccccagcccguggcucccgcugcugaucccggcgccggccccgggccugaccgugcagcugcugcucucgcugcugcugcugaugcccguccacccgcagcggcugccgcgcaugcaggaggacagcccgcucgggggcggcagcagcggcgaggacgacccgcugggcgaggaggaccugccgagcgaggaggacagcccccgggaggaggacccgccgggcgaggaggaccugccgggggaggaggaccugccgggcgaggaggaccugccggaggugaagcccaagucggaggaggagggcagccucaagcuggaggaccugccgacgguggaggccccgggcgacccgcaggagccgcagaacaacgcccaccgggacaaggagggcgacgaccagagccacuggcgguacggcggggacccgcccuggccgcgggugagcccggccugcgccggccgguuccagagcccgguggacauccgcccgcagcuggcggccuucugcccggcccugcggccccuggagcuccugggcuuccagcugccgccgcugccggagcugcggcugcggaacaacggccacagcgugcagcucacccugccgccgggccuggagauggcccugggccccgggcgggaguaccgggcccugcagcugcaccuccacuggggcgccgcgggccgcccgggcucggagcacaccgucgagggccaccgguucccggccgagauccacguggugcaccugagcaccgccuucgcccggguggacgaggcccugggccggccgggcgggcuggccgugcuggcggccuuccuggaggagggcccggaggagaacagcgccuacgagcagcuccugagccggcuggaggagaucgccgaggagggcagcgagacccaggugccgggccuggacaucagcgcccugcugcccucggacuucagccgguacuuccaguacgagggcagccucaccacgccgccgugcgcccagggggucaucuggaccguguucaaccagaccgugaugcugagcgcgaagcagcugcacacccugagcgacacccuguggggcccgggcgacagccgccugcagcucaacuuccgggccacccagccgcugaacggccgggugaucgaggccucguucccggccggcguggacagcagcccccgggccgccgagccggugcagcugaacagcugccuggcggccggcgacauccuggcccuggucuucgggcuccuguucgccgugacgagcguggccuuccuggugcagaugcggcggcagcaccgccggggcaccaagggcggcgugagcuaccggccggccgagguggcggagaccggcgcc

SEQ ID NO: 27 - RNA, 1629 nt, Artificial Sequence
  synthetic construct
augggcaccagccugucgccgaacgaccccuggccgcucaacccgcugagcauccagcagacgacccugcugcugcugcucagcgugcuggccaccguccacguggggcagcggcugcugcgccagcggcggcggcagcugcggagcgcgccgccgggcccguucgccuggccccugaucggcaacgccgccgccgugggccaggccgcgcaccucagcuucgcccggcuggcccgccgguacggcgacguguuccagauccggcugggcagcugcccgaucguggugcugaacggggagcgggccauccaccaggcccugguccagcagggcucggccuucgcggaccggccggccuucgccagcuuccggguggugagcggcggccgcagcauggccuucggccacuacagcgagcacuggaaggugcagcggcgggccgcccacagcaugaugcggaacuucuucacccggcagccgcggucgcgccaggugcuggagggccacgugcucagcgaggcgcgggagcuggucgcccugcuggugcgggggagcgccgacggcgccuuccuggacccgcggccgcugaccgugguggccguggccaacgugaugagcgcggucugcuucggcugccgguacagccacgacgaccccgaguuccgggagcuccugagccacaacgaggaguucggccgcaccgugggcgccggcucgcugguggacgugaugccguggcugcaguacuucccgaacccggugcggacgguguuccgggaguucgagcagcugaaccggaacuucagcaacuucauccuggacaaguuccuccggcacugcgagagccugcggccgggggccgccccgcgcgacaugauggacgccuucauccugagcgccgagaagaaggcggccggcgacagccacggcggcggcgcccggcuggaccuggagaacguccccgccaccaucaccgacaucuucggcgccagccaggacacccugucgaccgcccuccaguggcugcugcugcuguucacccgguacccggacgugcagacgcgggugcaggcggagcuggaccaggugguggggcgggaccggcucccgugcaugggcgaccagccgaaccugccguacgugcuggccuuccuguacgaggccaugcgcuucagcagcuucgucccggugaccaucccccacgccaccaccgccaacaccagcgugcugggcuaccacaucccgaaggacaccgugguguucgugaaccaguggagcgucaaccacgacccgcugaaguggccgaacccggagaacuucgacccggcccgguuccucgacaaggacggccugaucaacaaggaccugacgagccgggugaugaucuucucggugggcaagcggcggugcaucggcgaggagcugagcaagaugcagcuguuccuguucaucagcauccucgcgcaccagugcgacuuccgggccaaccccaacgagccggccaagaugaacuucagcuacgggcugaccaucaagccgaagagcuucaaggugaacgugacccugcgcgagagcauggagcugcuggacucggccgugcagaaccugcaggccaaggagaccugccag

SEQ ID NO: 28 - RNA, 885 nt, Artificial Sequence
  synthetic construct
auggagcaccagcugcucugcugcgagguggagaccauccggcgcgccuacccggacgcgaaccugcugaacgaccggguccugcgggccaugcugaaggccgaggagacgugcgcccccagcgugucguacuucaagugcgugcagaaggaggugcugccgagcaugcggaagaucguggccaccuggaugcucgaggugugcgaggagcagaagugcgaggaggaggucuucccgcuggccaugaacuaccuggaccgguuccugagccuggagccggugaagaagagccggcugcagcuccugggcgcgaccugcauguucguggccagcaagaugaaggagaccaucccgcugaccgccgagaagcugugcaucuacaccgacaacagcauccgcccggaggagcugcugcagauggagcuccugcuggugaacaagcugaaguggaaccuggccgccaugacgccccacgacuucaucgagcacuuccugucgaagaugccggaggccgaggagaacaagcagaucauccggaagcacgcgcagaccuucguggcccucugcgccaccgacgugaaguucaucagcaacccgccgagcauggucgccgccgggagcgugguggccgcggugcagggccugaaccugcggagcccgaacaacuuccugagcuacuaccggcugacccgguuccugucgcgggugaucaagugcgacccggacugccuccgcgccugccaggagcagaucgaggcccugcuggagagcagccugcggcaggcccagcagaacauggaccccaaggccgccgaggaggaggaggaggaggaggaggagguggaccuggcgugcaccccgaccgacguccgggacguggacauc

SEQ ID NO: 29 - RNA, 783 nt, Artificial Sequence
  synthetic construct
auguucgaggcccggcuggugcagggcagcauccucaagaagguccuggaggcgcugaaggaccugaucaacgaggccugcugggacaucucgagcagcggggugaaccugcagagcauggacagcagccacgugucgcuggugcagcucacccugcgcagcgagggcuucgacacguaccggugcgaccggaaccuggccaugggcgugaaccugaccagcaugagcaagauccugaagugcgccggcaacgaggacaucaucacccugcgggccgaggacaacgccgacacccucgcgcugguguucgaggccccgaaccaggagaaggucagcgacuacgagaugaagcugauggaccuggacguggagcagcugggcauccccgagcaggaguacagcugcguggugaagaugccgucgggcgaguucgcccggaucugccgggaccugagccacaucggggacgccguggugaucagcugcgccaaggacggcgucaaguucagcgccagcggcgagcucggcaacggcaacaucaagcugagccagaccucgaacguggacaaggaggaggaggcggugaccaucgagaugaacgagccggugcagcugacguucgcccugcgcuaccugaacuucuucaccaaggccaccccgcugagcagcaccgugacccucagcaugagcgccgacgugccgcuggucguggaguacaagaucgccgacaugggccaccugaaguacuaccuggccccgaagaucgaggacgaggaggggagc

SEQ ID NO: 30 - RNA, 1041 nt, Artificial Sequence
  synthetic construct
augccgcggggccagaagagcaagcugcgcgcccgggagaagcggcggaaggcgcgggaggagacccaggggcucaaggugcggcacgccacggccgccgagaaggaggagugccccucgagcagcccgguccugggcgacaccccgaccagcagcccggccgccggcaucccgcagaagccgcagggcgcgcccccgaccaccaccgccgccgccgccgugagcugcacggagucggacgagggcgccaagugccagggcgaggagaacgcgagcuucagccaggccaccaccagcaccgagagcagcgugaaggacccgguggccugggaggccgggaugcugaugcacuucauccugcgcaaguacaagaugcgggagccgaucaugaaggccgacaugcugaaggugguggacgagaaguacaaggaccacuucaccgagauccugaacggcgccucgcggcggcucgagcuggucuucggccuggaccugaaggaggacaacccgagcagccacaccuacacgcuggugagcaagcugaaccucaccaacgacggcaaccugagcaacgacugggacuucccgcggaacggccugcugaugccccugcugggcgugaucuuccucaaggggaacagcgcgaccgaggaggagaucuggaaguucaugaacgugcugggcgccuacgacggcgaggagcaccugaucuacggcgagccgcggaaguucaucacccaggaccuggugcaggagaaguaccugaaguacgagcaggugccgaacucggacccgccgcgcuaccaguuccuguggggcccgcgggccuacgccgagaccaccaagaugaagguccucgaguuccuggccaagaugaacggcgccacgccccgggacuucccgagccacuacgaggaggcgcugcgggacgaggaggagcgggcccaggugcggagcagcgugcgcgcccggcggcggaccaccgccaccaccuuccgggcccggagccgcgccccguucagccggucgagccacccgaug

SEQ ID NO: 31 - RNA, 2361 nt, Artificial Sequence
  synthetic construct
augagcaccaacccgaagccccagcggaagacgaagcgcaacaccaaccggcggccgcaggacgugaaguucccgggcgggggccagaucgucggcggcguguaccugcucccgcggcggggcccgcggcugggcgugcgcgccacccggaagaccucggagcggagccagccgcgggggcggcggcagcccaucccgaaggcgcgccagccggagggccgggccugggcccagccgggcuacccguggccgcuguacggcaacgagggccugggcugggccggguggcugcugagcccccggggcagccggccgagcuggggcccgaccgacccgcggcggcgcagccggaaccucggcaaggugaucgacacccugacgugcggcuucgccgaccugaugggcuacaucccgcuggugggggccccgcugggcggcgcggcccgggcccuggcccacggcgugcggguccucgaggacggcgugaacuacgccaccggcaaccugcccgggugcucguucagcaucuuccugcuggcccugcugagcugccucaccaucccggcgagcgccuacgaggugcggaacgugagcggcguguaccacgugaccaacgacugcagcaacucgagcaucgucuacgaggccgccgacaugaucaugcacaccccgggcugcgugccgugcgugcgggagaacaacuucagccgcugcuggguggcccugaccccgacgcuggccgcgcggaacagcagcaucccgaccaccaccauccggcggcacaucgaccugcuggugggcgccgccgccuucugcagcgccauguacgugggcgaccugugcggcucggucuuccucgugagccagcuguucaccuucagcccccggcgguacgagaccgugcaggacugcaacugcagcaucuacccggggcacgugagcggccaccgcauggccugggacaugaugaugaacuggagcccgacgaccgcgcugguggugucgcagcugcugcggauccggcaggccguccuggacaugguggccggcgcccacuggggcgugcucgccggccuggccuacuacagcauggugggcaacugggcgaaggugcugaucgugaugcugcuguucgccggggucgacggcgcccagcggcuggacacccgggugaccggcgcccaggccggccggaccacccagggcuuccgcagccucuucaugccgggccaggagcagaagauccagcugauccacacgaacgggagcuggcacaucaaccggaccgcccugaacugcaacgacagccugcagaccggcuuccuggcggcccuguucuacacccacagcuucaacucgagcggcggcccggagcggauggcccagugccggccgaucgaccgguucgcccagggcuggggccccaucacccacgacgccagcgggaaccucgaccagcggccguacugcagcagcuacgccccgaagccgugcggcaucgugccggcgagccaggugugcggcccgguguacugcuucacccccucgccgguggucgugggcacgaccgaccgcuucggcgugccgaccuacagcuggggcgccaacgagaccgacgugcugcugcugaacaacacccugccgccgcaggggaacugguucggcugcaccuggaugaacggcacgggcagcaccaagaccugcggccggccgcccugcaagaucggcggggugggcaacaacacccugaucugcccgaccgacugcuuccggaagcacccggaggccaccuacacgaagugcggcagcggcccguggcuccggccgcggugcaugguggacuacccguaccggcuguggcacuaccccugcaccgucaacuucagcguguucaaggugcgcauguacgugggcggcguggagcagcggcugaacgccgccugcaacuggacccggggcgagcggugcgaccuggaggaccgggaccggagcgagcugucgccgcugcuccugagcaccaccgaguggcagauccugccgugcagcuucaccacgcugccggcccugagcaccgggcugauccaccuccaccgcaacaucguggacguccaguaccuguacggcgugggcagcgugguggugagcuucgcgaucaagugggaguacaucgugcugcuguuccugcugcucgccgacgcccgggucugcgccugccuguggaugaugcugcugguggcccaggccgaggcggcccuggagaaccugguggugcucaacgccgccuacguggccggcgcccacggcauccugucguuccugguguucuucugcgcggccugguacaucaaggggaagcugguc

SEQ ID NO: 32 - RNA, 1179 nt, Artificial Sequence
  synthetic construct
auggaggagccgcagagcgaccccucgguggagccgccgcugagccaggagaccuucagcgaccucuggaagcugcugccggagaacaacguccugagcccgcugccgagccaggccauggacgaccugaugcucagccccgacgacaucgagcagugguucacggaggacccgggcccggacgaggcgccgcggaugccggaggccgccccgcccguggccccggccccggccaccccgaccccggcggccccggcccccgccccgucguggccgcugagcagcagcgugccgagccagaagaccuaccaggggagcuacggcuuccgccugggcuuccugcacucgggcaccgccaagagcgugaccugcacguacagcccggcccugaacaagauguucugccagcuggcgaagaccugcccggugcagcucuggguggacagcaccccgccgcccggcacccggguccgggccauggccaucuacaagcagagccagcacaugaccgagguggugcggcggugcccgcaccacgagcggugcagcgacucggacggccuggccccgccgcagcaccugauccgcguggaggggaaccugcggguggaguaccuggacgaccggaacaccuuccggcacagcguggucgugcccuacgagccgccggaggugggcagcgacugcacgaccauccacuacaacuacaugugcaacagcagcugcaugggcggcaugaaccggcggccgauccugaccaucaucacccucgaggacagcucgggcaaccugcugggccgcaacagcuucgaggugcgggugugcgccugcccggggcgggaccggcggaccgaggaggagaaccugcggaagaagggcgagccgcaccacgagcugcccccgggcagcaccaagcgcgcccugccgaacaacacgagcagcagcccgcagccgaagaagaagccgcucgacggcgaguacuucacccugcagauccggggccgggagcgguucgagauguuccgggagcugaacgaggcgcuggagcugaaggacgcccaggccggcaaggagcccgggggcucgcgggcccacagcagccaccugaagagcaagaagggccagagcaccagccgccacaagaagcucauguucaagaccgagggcccggacucggac

SEQ ID NO: 33 - RNA, 1527 nt, Artificial Sequence
  synthetic construct
auggagcggcgccggcuguggggcagcauccagucgcgguacaucagcaugagcguguggaccagcccgcggcggcucgucgagcuggccgggcagagccugcugaaggacgaggcgcuggccaucgccgcccuggagcuccugccccgggagcuguucccgccgcuguucauggccgccuucgacggccgccacagccagacgcugaaggcgauggugcaggccuggccguucaccugccugccgcucggcgugcugaugaagggccagcaccugcaccuggagaccuucaaggccgugcuggacggccuggacgugcuccuggcccaggaggugcggccgcggcgguggaagcugcagguccuggaccugcggaagaacucgcaccaggacuucuggaccguguggagcggcaaccgggccagccuguacagcuuccccgagccggaggccgcgcagccgaugaccaagaagcgcaagguggacgggcucagcaccgaggccgagcagccguucaucccgguggaggugcugguggaccuguuccugaaggagggcgccugcgacgagcuguucagcuaccugaucgagaaggucaagcggaagaagaacgugcuccggcugugcugcaagaagcugaagaucuucgccaugccgaugcaggacaucaagaugauccugaagauggugcagcuggacucgaucgaggaccuggaggugacgugcaccuggaagcuccccacccuggccaaguucagcccguaccugggccagaugaucaaccugcggcggcugcugcucagccacauccacgccagcagcuacaucagcccggagaaggaggagcaguacaucgcgcaguucaccucgcaguuccugagccugcagugccugcaggcccuguacguggacagccuguucuuccuccggggccgccuggaccagcugcugcggcacgugaugaacccgcuggagacccugagcaucaccaacugccggcucagcgagggcgacgucaugcaccugagccagucgccgagcgugagccagcugagcgugcugagccugagcggcgugaugcugacggacgugucgccggagccccuccaggcccugcuggagcgggccagcgccacccugcaggaccugguguucgacgagugcgggaucaccgacgaccagcugcucgcccugcugccgagccugagccacugcagccagcugaccacccugagcuucuacggcaacucgaucagcaucagcgcgcuccagagccugcugcagcaccugaucggccugagcaaccugacccacguccucuacccggugccgcuggagagcuacgaggacauccacggcacgcugcaccuggagcggcuggccuaccugcacgcccggcuccgcgagcugcugugcgagcugggccggccgucgaugguguggcugagcgccaacccgugcccccacugcggcgaccggaccuucuacgacccggagccgauccugugcccgugcuucaugccgaac

SEQ ID NO: 34 - RNA, 219 nt, Artificial Sequence
  synthetic construct
auggagcugccgaagcucaagcugagcggcgugguccggcugucgagcuacagcagccccacccugcagagcgugcuggggagcggcacgaacggccgcgugccggugcugcggccgcucaagugcaucccggccucgaagaagaccgacccgcagaaggaccugaagccggcgccccagcagugccggcugccgaccaucgugcggaagggcggccgg

SEQ ID NO: 35 - RNA, 1467 nt, Artificial Sequence
  synthetic construct
augugcaacaccaacaugagcgugucgacggagggcgccgcgagcaccagccagaucccggccagcgagcaggagacccugguccggcccaagccgcuccugcugaagcugcugaagagcgugggggcccagaacgacaccuacaccaugaaggagaucaucuucuacaucggccaguacaucaugaccaagcgccuguacgacgagaagcagcagcacaucguguacugcagcaacgaccuccugggcgacguguucggcgugccgucguucagcgugaaggagcaccggaagaucuacgccaugaucuaccggaaccuggucgccgugagccagcaggacagcggcacgagccugagcgagucgcggcggcagccggagggcgggagcgaccugaaggacccgcugcaggccccgcccgaggagaagccgagcagcagcgaccucaucagccggcugucgaccagcagccgccggcggagcaucagcgagaccgaggagaacaccgacgagcugccgggcgagcggcaccggaagcggcgccggagccugucguucgacccgagccugggccugugcgagcuccgggagaugugcagcggcggcaccagcagcagcucgagcagcagcagcgagagcaccgagacgccgucgcaccaggaccuggacgacggcgugagcgagcacagcggggacugccuggaccaggacagcgugagcgaccaguucagcguggaguucgagguggagucgcuggacagcgaggacuacagccugagcgacgagggccacgagcugagcgacgaggacgacgaggucuaccgggugaccguguaccagaccggcgagagcgacaccgacucguucgagggcgacccggagaucagccucgcggacuacuggaagugcaccagcugcaacgagaugaacccgccgcugcccagccacugcaagcggugcuggacccugcgggagaacuggcugccggacgacaagggcaaggacaagguggagaucagcgagaaggccaagcuggagaacagcgcccaggccgaggagggccuggacgugccggacgggaagaagcucacggagaacgacgccaaggagccgugcgccgaggaggacucggaggagaaggcggagcagaccccgcugagccaggagagcgacgacuacagccagcccagcaccagcucgagcaucguguacagcagccaggagagcgucaaggagcugaaggaggagacccagcacaaggacgagagcguggagucgagcuucagccugaacgccaucgagccgugcgugaucugccagggccgcccgaagaacggcugcaucgugcacggcaagaccggccaccugaugagcugcuucaccugcgccaagaagcugaagaagcggaacaagccgugcccggugugccggcagcccauccagaugaucgugcucagcuacuucaac

SEQ ID NO: 36 - RNA, 2064 nt, Artificial Sequence
  synthetic construct
auggacaaggugcugaaccgggaggagagcauggagcucauggaccugcugggccuggaccgcucggccugggggaacaucccggucaugcggaaggcguaccugaagaagugcaaggagcugcaccccgacaagggcggcgacgaggacaagaugaagcggaugaacuuccucuacaagaagauggagcagggcgugaagguggcccaccagccggacuucggcaccuggaacagcagcgaggugccgacguacggcaccgacgagugggagagcugguggaacaccuucaacgagaagugggacgaggaccuguucugccacgaggagauguucgccagcgacgacgagaacaccgggagccagcacucgaccccgccgaagaagaagaagaagguggaggacccgaaggacuuccccguggaccugcacgccuuccugagccaggccgucuucagcaaccggaccguggccagccuggcgguguacacgaccaaggagaaggcccagauccuguacaagaagcucauggagaaguacagcgugaccuucaucagccggcacggcuucggcggccacaacauccuguucuuccugaccccgcaccggcaccgcgugucggccaucaacaacuacugccagaagcugugcaccuucagcuuccugaucugcaagggcgugaacaaggaguaccuguucuacagcgcccucugccggcagccguacgccgucguggaggagagcauccagggcgggcugaaggagcacgacuucaacccggaggagccggaggagaccaagcaggugagcuggaagcuggugacgcaguacgcccuggagaccaagugcgaggacguguuccugcugaugggcauguaccucgacuuccaggagaacccgcagcagugcaagaagugcgagaagaaggaccagcccaaccacuucaaccaccacgagaagcacuacuacaacgcgcagaucuucgccgacagcaagaaccagaagucgaucugccagcaggccguggacaccgucgccgccaagcagcggguggacagcauccacaugacccgggaggagaugcugguggagcgguucaacuuccugcuggacaagauggaccugaucuucggcgcccacggcaacgcggugcuggagcaguacauggccggcguggccuggauccacugccuccugccgcagauggacaccgugaucuacgacuuccugaagugcaucguccugaacaucccgaagaagcgguacuggcuguucaagggcccgaucgacagcgggaagaccacgcuggccgccgcccuccuggaccugugcggcggcaagagccugaacgugaacaugccgcuggagcgccugaacuucgagcucggcgugggcaucgaccaguucauggugguguucgaggacgugaagggcaccggggcggagagccgggaccugccgagcggccacggcaucucgaaccuggacugccugcgggacuaccuggacggcagcgucaaggugaaccuggagcggaagcaccagaacaagcggacccagguguucccgcccggcaucgugaccaugaacgaguacagcgugccgcggacccuccaggcccgcuucgugcggcagaucgacuuccggccgaaggccuaccugcggaagagccugagcugcagcgaguaccugcuggagaagcggauccugcagucgggcaugacccuccugcugcugcugaucugguuccggccggucgccgacuucgccgccgcgauccacgagcgcaucgugcaguggaaggagcggcuggaccucgagaucagcauguacacguucagcaccaugaaggccaacguggggaugggccggccgauccuggacuucccccgggaggaggacagcgaggccgaggacagcggccacggcagcucgaccgagagccagagccagugcagcagccaggugagcgaggccucgggcgccgacacccaggagaacugcaccuuccacaucugcaagggcuuccagugcuucaagaagccgaagaccccgccgccgaag

SEQ ID NO: 37 - RNA, 231 nt, Artificial Sequence
  synthetic construct
augauccuguuccagagcaacaccacgaccucguacgccuacaccaacauccagccgaaguacgcgaugcagcucgagaucaccauccugaucgugaucggcauccugauccugagcgucauccuguacuucaucuucugccggcagauccccaacgugcaccgcaacagcaagcggcggccgaucuacagcccgaugaucagccggccgcacauggcccugaacgagauc

SEQ ID NO: 38 - RNA, 2652 nt, Artificial Sequence
  synthetic construct
augggcgacuacauggcccaggaggacgacugggaccgggaccugcuccuggacccggcgugggagaagcagcagcgcaagaccuucacggccuggugcaacagccaccugcggaaggccgggacccagaucgagaacaucgacgaggacuuccgggacggccugaagcugaugcugcuccuggaggugaucucgggcgagcggcugcccaagccggagcggggcaagaugcggguccacaagaucaacaacgugaacaaggcccuggacuucaucgccagcaagggcgugaagcuggugagcaucggcgccgaggagaucguggacgggaacgcgaagaugacccugggcaugaucuggaccaucauccuccgcuucgccauccaggacaucagcguggaggagaccagcgccaaggagggccugcugcuguggugccagcggaagaccgccccguacaagaacgucaacgugcagaacuuccacaucagcuggaaggacggccuggccuucaacgcccugauccaccggcaccggccggagcucaucgaguacgacaagcugcggaaggacgacccggugacgaaccugaacaacgcguucgagguggccgagaaguaccuggacaucccgaagaugcuggacgccgaggacaucgugaacaccgcccggcccgacgagaaggccaucaugaccuacgugucgagcuucuaccacgccuucagcggcgcgcagaaggccgagaccgccgccaaccgcaucugcaagguccuggccgugaaccaggagaacgagcaccucauggaggacuacgagaagcuggccagcgaccugcuggaguggauccggcggaccaucccguggcuggaggaccgggugccgcagaagaccauccaggagaugcagcagaagcuggaggacuuccgggacuaccggcgcgugcacaagccgccgaaggugcaggagaagugccagcucgagaucaacuucaacacgcugcagaccaagcugcggcugagcaaccggccggcguucaugcccagcgagggcaagauggugucggacaucaacaacggguggcagcaccuggagcaggccgagaagggcuacgaggaguggcugcucaacgagauccggcggcuggagcggcuggaccaccuggccgagaaguuccgccagaaggccagcauccacgaggccuggaccgacggcaaggaggccaugcugaagcaccgggacuacgagaccgcgacccugagcgacaucaaggcccucauccggaagcacgaggccuucgagagcgaccuggccgcccaccaggaccgggucgagcagaucgccgcgaucgcccaggagcugaacgagcuggacuacuacgacagccacaacgugaacacccggugccagaagaucugcgaccagugggacgcccugggcagccugacgcacucgcggcgcgaggcccucgagaagaccgagaagcagcuggaggccaucgaccagcugcaccuggaguacgccaagcgggcggccccguucaacaacuggauggagagcgccauggaggaccugcaggacauguucaucgugcacaccaucgaggagaucgagggccugaucagcgcccacgaccaguucaagagcacccucccggacgccgaccgggagcgggaggccauccuggcgauccacaaggaggcccagcggaucgccgagagcaaccacaucaagcugagcggcucgaacccguacaccaccgugacgccgcagaucaucaacagcaagugggagaaggugcagcagcuggugccgaagcgggaccacgcccugcuggaggagcagagcaagcagcagagcaacgagcaccuccgccggcaguucgccagccaggccaacgucguggggcccuggauccagaccaagauggaggagaucggccggaucagcaucgagaugaacggcacccuggaggaccagcugucgcaccugaagcaguacgagcggagcaucguggacuacaagccgaaccuggaccugcucgagcagcagcaccagcugauccaggaggcgcugaucuucgacaacaagcacaccaacuacaccauggagcacauccgggugggcugggagcagcugcugaccacgaucgcccggaccaucaacgagguggagaaccagauccugacccgcgacgccaagggcaucagccaggagcagaugcaggaguuccgggccagcuucaaccacuucgacaaggaccacggcggggcccucggcccggaggaguucaaggccugccugaucagccugggcuacgacguggagaacgaccggcagggcgaggcggaguucaaccggaucaugagccuggucgacccgaaccacucgggccuggugaccuuccaggccuucaucgacuucaugagccgggagaccaccgacacggacaccgccgaccaggugaucgccagcuucaaggugcuggccggcgacaagaacuucaucaccgccgaggagcuccggcgcgagcugccgccggaccaggcggaguacugcaucgcccggauggcccccuaccaggggccggacgccgugccgggcgcccuggacuacaagagcuucagcaccgcccuguacggcgagagcgaccug

SEQ ID NO: 39 - RNA, 327 nt, Artificial Sequence
  synthetic construct
augcugauggcccaggaggcgcucgccuuccugauggcccagggcgccaugcuggccgcccaggagcggcgcgugccgcgggcggccgagguccccggggcccagggccagcagggcccgcggggccgggaggaggccccgcggggcgugcggauggccgugccgcugcugcgccggauggagggcgccccggcggggccgggcggccggaccgccgccugcuucagcugcacgucgcggugccugagccggcggcccuggaagcgcagcuggagcgccggcagcugcccgggcaugccgcaccucagcccggaccagggccgguuc

SEQ ID NO: 40 - RNA, 630 nt, Artificial Sequence
  synthetic construct
augcaggccgagggccaggggaccggcggcagcacgggcgacgcggacggcccgggcgggcccggcaucccggacggcccgggcggcaacgccggcgggccgggcgaggccggcgccaccggcggccggggcccgcgcggggccggcgccgcgcgggccucgggcccgcggggcggcgccccgcggggcccccacgggggcgccgccagcgcccaggacggccggugcccgugcggcgcgcggcgcccggacagccggcugcuccagcugcacaucaccaugccguucagcagcccgauggaggccgagcuggugcggcggauccugagccgggacgccgccccgcugccccggccgggcgccguccugaaggacuucaccgugucgggcaaccuccuguucaugagcgugcgcgaccaggaccgggagggggccggccggaugcggguggugggcuggggccugggcagcgcgagcccggagggccagaaggcccgggaccugcggaccccgaagcacaaggugagcgagcagcgcccggggacccccggcccgccgccgccggagggcgcccagggcgacggcugccggggcgucgccuucaacgugauguucagcgccccgcacauc

SEQ ID NO: 41 - RNA, 909 nt, Artificial Sequence
  synthetic construct
auggccaccagccgguacgagccgguggcggagaucggcgucggggccuacggcacgguguacaaggcccgcgacccccacucgggccacuucguggcccugaagagcgugcgggugccgaacggcggcggcgggggcggcggccucccgaucagcaccgugcgggaggucgcccugcugcggcggcuggaggccuucgagcacccgaacguggugcggcugauggacgugugcgcgaccagccgcaccgaccgggagaucaaggugacccugguguucgagcacgucgaccaggaccuccggaccuaccuggacaaggccccgccgcccggccugccggccgagacgaucaaggaccugaugcggcaguuccugcggggccuggacuuccuccacgccaacugcaucgugcaccgggaccugaagccggagaacauccuggugaccagcgggggcaccgugaagcuggccgacuucggccuggcccgcaucuacagcuaccagauggcgcugaccccggugguggucacccucugguaccgggccccggaggugcugcugcagucgaccuacgccacgccgguggacauguggagcgugggcugcaucuucgccgagauguuccggcggaagccccuguucugcggcaacagcgaggccgaccagcugggcaagaucuucgaccugaucgggcucccgccggaggacgacuggccgcgggacgugagccugccgcggggcgccuucccgccccgcggcccgcggccggugcagagcgucgugccggagauggaggagagcggcgcgcagcugcugcuggagaugcugaccuucaacccgcacaagcggaucucggccuuccgggcccuccagcacagcuaccugcacaaggacgagggcaacccggag

SEQ ID NO: 42 - RNA, 1524 nt, Artificial Sequence
  synthetic construct
augugggagauccugcggcgcaaggacugcgacaaggagaagcgggugaagcucaugagcgaccugcagaagcugauccagggcaagaucaagaccaucgccuucgcgcacgacucgacgcgggucauccagugcuacauccaguacgggaacgaggagcagcggaagcaggccuucgaggagcugcgggacgaccugguggagcugagcaaggccaaguacagccggaacaucgugaagaaguuccucauguacggcagcaagccgcagaucgccgagaucauccgcagcuucaagggccacgugcggaagaugcugcggcacgccgaggccagcgcgaucguggaguacgccuacaacgacaaggccauccuggagcagcggaacaugcugaccgaggagcuguacggcaacaccuuccagcuguacaagucggccgaccaccggacccucgacaaggugcuggagguccagcccgagaagcuggagcugaucauggacgagaugaagcagauccugaccccgauggcccagaaggaggccgugaucaagcacagccuggugcacaagguguuccucgacuucuucaccuacgcgccgccgaagcugcggagcgagaugaucgaggccauccgcgaggccgugguguaccuggcccacacgcacgacggcgcccgggucgccaugcacugccuguggcacggcaccccgaaggaccggaaggugaucgugaagaccaugaagaccuacguggagaagguggcgaacgggcaguacagccaccuggugcugcucgccgccuucgacugcaucgacgacaccaagcuggucaagcagaucaucaucagcgagaucaucagcucgcugccgagcaucgugaacgacaaguacggccggaaggugcugcuguaccugcucagcccccgggacccggcccacaccgugcgggagaucaucgaggugcugcagaagggcgacggcaacgcccacagcaagaaggacacggaggugcgccggcgggagcugcuggagagcaucagcccggcccugcugucguaccuccaggagcacgcgcaggaggucgugcuggacaagagcgccugcgugcuggugagcgacauccugggcagcgccaccggcgacgugcagccgaccaugaacgccaucgccagccuggccgcgaccgggcugcacccgggcggcaaggacggcgagcuccacaucgccgagcacccggccggccaccuggugcugaaguggcugaucgagcaggacaagaagaugaaggagaacggccgggaggggugcuucgccaagacccuggucgagcacgugggcaugaagaaccugaagagcugggccucggugaaccggggcgccaucauccucagcagccugcugcagagcugcgaccuggagguggcgaacaaggugaaggccgcccugaagagccugauccccacccucgagaagacgaagagcaccucgaagggcaucgagauccugcuggagaagcugagcacc

SEQ ID NO: 43 - RNA, 942 nt, Artificial Sequence
  synthetic construct
augccgcuggagcagcggagccagcacugcaagcccgaggagggccucgaggcccgcggggaggcgcugggccuggugggcgcccaggccccggccaccgaggagcagcagacggccucgagcagcagcacccuggucgaggugacccugggcgaggugccggccgcggacagcccgagcccgccgcacucgccccagggcgccagcagcuucagcaccaccaucaacuacacccuguggcggcagagcgacgagggcagcucgaaccaggaggaggaggggccgcggauguucccggaccucgagagcgaguuccaggccgccaucagccggaagaugguggagcuggugcacuuccugcugcugaaguaccgggcccgggagccggugacgaaggccgagaugcuggagagcguccuccgcaacugccaggacuucuucccggugaucuucagcaaggcgagcgaguaccugcagcugguguucggcaucgaggugguggagguggucccgaucucgcaccuguacauccuggugaccugccugggccucagcuacgacggccugcugggcgacaaccaggugaugcccaagaccggccugcugaucaucgugcuggccaucaucgccaucgagggggacugcgccccggaggagaagaucugggaggagcucagcaugcuggagguguucgagggccgggaggacagcguguucgcccacccgcggaagcugcugaugcaggaccugguccaggagaacuaccuggaguaccggcaggugccgggcagcgacccggccugcuacgaguuccucuggggcccgcgggcgcugaucgagaccagcuacgugaaggugcugcaccacacccugaagaucggcggcgagccccacaucucguacccgccgcugcacgagcgggcccugcgcgagggggaggag

SEQ ID NO: 44 - RNA, 951 nt, Artificial Sequence
  synthetic construct
augagcucggagcagaagagccagcacugcaagccggaggagggcguggaggcccaggaggaggcgcuggggcucgucggcgcccaggcccccaccacggaggagcaggaggccgccgugagcagcagcagcccgcuggugccgggcacccuggaggaggugccggccgcggagucggccggcccgccgcagagcccccagggcgccagcgcccugccgaccaccaucagcuucaccugcuggcggcagccgaacgagggcagcagcucgcaggaggaggaggggccgagcaccagcccggacgccgagagccuguuccgcgaggcccugagcaacaagguggacgagcucgcgcacuuccugcugcggaaguaccgggccaaggagcuggugacgaaggccgagaugcuggagcgggucaucaagaacuacaagcggugcuucccggugaucuucggcaaggccagcgagucgcugaagaugaucuucggcaucgacgugaaggagguggaccccgccagcaacaccuacacccucgugaccugccugggccugagcuacgacggccugcugggcaacaaccagaucuucccgaagaccgggcugcucaucaucgugcugggcaccaucgccauggagggcgacagcgcgagcgaggaggagaucugggaggagcugggcgucaugggcguguacgacggccgggagcacacgguguacggggagccgcgcaagcugcugacccaggacugggugcaggagaacuaccuggaguaccggcaggugccgggcagcaacccggcccgguacgaguuccucuggggcccgcgggcccuggccgagaccucguacgugaagguccuggagcacguggugcgggugaacgcccgggugcgcaucgccuaccccagccugcgggaggcggcccugcuggaggaggaggagggcgug

SEQ ID NO: 45 - RNA, 951 nt, Artificial Sequence; identical to SEQ ID NO: 44
  synthetic construct

SEQ ID NO: 46 - RNA, 951 nt, Artificial Sequence
  synthetic construct
auggccgugcagggcagccagcggcgccugcucgggucgcugaacagcaccccgacggcgaucccccagcugggccuggccgccaaccagaccggcgcccggugccuggaggucagcaucagcgacggccuguuccucagccugggccuggugagccugguggagaacgcccuggugguggccaccaucgcgaagaaccggaaccugcacucgccgauguacugcuucaucugcugccucgcccugagcgaccugcuggugagcggcaccaacguccuggagaccgccgugauccugcuccuggaggccggggcccugguggcccgggcggccgugcugcagcagcuggacaacgugaucgacgugaucaccugcagcagcaugcugagcucgcucugcuuccugggcgccaucgccgucgaccgguacaucagcaucuucuacgcccugcgguaccacagcaucgugacgcugccgcgcgccccgcgggcgguggccgccaucuggguggccagcgugguguucagcacccuguucaucgccuacuacgaccacgucgccgugcugcucugccuggugguguucuuccuggcgaugcuggugcugauggccgugcuguacguccacaugcucgcccgggccugccagcacgcccagggcaucgcccggcugcacaagcggcagcggccggugcaccagggcuucggccugaagggcgcggugacccugaccauccugcuggggaucuucuuccucugcuggggcccguucuuccugcaccugacccugaucgugcugugccccgagcacccgaccugcggcugcaucuucaagaacuucaaccuguuccucgcccugaucaucugcaacgccaucaucgacccgcugaucuacgccuuccacagccaggagcugcgccggacgcugaaggaggugcugaccugcucgugg

SEQ ID NO: 47 - RNA, 2850 nt, Artificial Sequence
  synthetic construct
uucgagaagggccggaucuacaccuacaucggggaggugcuggucagcgugaacccguaccaggagcucccccuguacggcccggaggccaucgcgcgcuaccagggccgggagcuguacgagcggccgccgcaccuguacgccguggccaacgccgccuacaaggccaugaagcaccggucgcgggacacgugcaucgugaucagcggcgagagcggcgcgggcaagaccgaggccagcaagcacaucaugcaguacaucgccgccgugaccaacccgagccagcgggccgagguggagcgcgucaaggacgugcugcugaagagcaccugcgugcucgaggccuucgggaacgcgcggaccaaccggaaccacaacucgagccgguucggcaaguacauggacaucaacuucgacuucaagggcgacccgaucggcggccacauccacagcuaccugcuggagaagagccgggugcugaagcagcacgugggcgagcggaacuuccacgccuucuaccagcugcugcgcgggagcgaggacaagcagcuccacgagcugcaccuggagcggaaccccgccguguacaacuucacccaccagggcgccggccugaacaugacgguccacagcgcccuggacucggacgagcagagccaccaggccgugaccgaggcgaugcgggugaucggcuucagcccggaggagguggagagcgugcaccggauccuggccgccauccuccaccugggcaacaucgaguucguggagaccgaggagggcgggcugcagaaggagggccuggccgucgccgaggaggcccugguggaccacguggcggagcugaccgccaccccgcgggaccucgugcugcggagccugcuggcccgcaccguggccagcggcggccgggagcugaucgagaagggccacacggccgccgaggcgucguacgcccgggacgccugcgccaaggccguguaccagcggcuguucgagugggucgugaaccggaucaacagcgugauggagcuccggggccgcgacccgcggcgggacgggaaggacaccgugaucggcgugcuggacaucuacggcuucgagguguucccggucaacagcuucgagcaguucugcaucaacuacugcaacgagaagcugcagcagcuguucauccagcugauccugaagcaggagcaggaggaguacgagcgggagggcaucaccuggcagagcguggaguacuucaacaacgccaccaucguggaccucguggagcggccgcaccggggcauccuggcggugcuggacgaggccugcagcagcgccggcaccaucaccgaccgcaucuuccugcagacgcuggacacccaccaccggcaccaccugcacuacaccucgcggcagcucugccccaccgacaagaccauggaguucgggcgggacuuccggaucaagcacuacgccggcgacgugaccuacagcgucgagggcuucaucgacaagaaccgggacuuccuguuccaggacuucaagcgccugcuguacaacagcacggacccgacccugcgggccauguggccggacggccagcaggacaucaccgaggugaccaagcggccgcugaccgccggcacccucuucaagaacagcaugguggcgcugguggagaaccuggccagcaaggagccguucuacgugcggugcaucaagccgaacgaggacaagguggccggcaagcuggacgagaaccacugccggcaccaggucgccuaccuggggcugcucgagaacgugcgggugcgccgggccggcuucgccagccggcagcccuacucgcgguuccugcugcgguacuggcaccugacgccgaucaccccgugggcgaucgugccggugauggccgugcagggcagccagcggcgccugcugggcagccucaacagcaccccgaccgccaucccgcagcugggccuggccgccaaccagaccggcgcccggugccuggaggucagcaucagcgacgggcuguuccugucgcucggccuggugagccugguggagaacgcgcuggugguggccaccaucgccaagaaccggaaccugcacagccccauguacugcuucaucugcugccuggcccucagcgaccugcuggugagcggcacgaacguccuggagaccgccgugauccugcugcucgaggccggcgcgcugguggcccgggccgccgugcugcagcagcuggacaacgugaucgacgugaucaccugcagcucgaugcugagcagccugugcuuccucggcgccaucgccgucgaccgguacaucagcaucuucuacgcgcugcgguaccacagcaucgugacccugccgcgcgccccgcgggccguggccgccaucuggguggccagcgugguguucucgacccuguucaucgcguacuacgaccacgucgccgugcugcugugccucgugguguucuuccuggccaugcuggugcugauggccgugcuguacguccacaugcuggcccgggccugccagcacgcgcagggcaucgcccggcuccacaagcggcagcggccggugcaccagggguucggccugaagggcgccgugacccugacgauccugcugggcaucuucuuccugugcuggggcccguucuuccuccaccugacccugaucgugcugugcccggagcaccccaccugcggcugcaucuucaagaacuucaaccuguuccuggcccucaucaucugcaacgccaucaucgacccgcugaucuacgccuuccacagccaggagcugcgccggacccugaaggaggugcugaccugcagcugg

SEQ ID NO: 48 - RNA, 2004 nt, Artificial Sequence
  synthetic construct
auggaccuggugcucaagcggugccugcugcaccuggccgucaucggcgcgcugcuggccgugggggccaccaaggugccgcgcaaccaggacuggcucggcgugagccggcagcugcggacgaaggccuggaaccggcagcuguaccccgaguggaccgaggcccagcggcuggacugcuggcggggcggccaggugucgcugaaggugagcaacgacggcccgacccugaucggcgccaacgcgagcuucagcaucgcccucaacuucccggggagccagaagguccugccggacggccaggugaucugggugaacaacaccaucaucaacggcagccagguguggggcggccagccgguguacccgcaggagaccgacgacgccugcaucuuccccgacggcgggccgugcccgucgggcagcuggagccagaagcgcagcuucguguacgucuggaagaccuggggccaguacuggcaggugcugggcggcccggugagcggccugagcaucgggacgggccgggccaugcugggcacccacaccauggaggugaccguguaccaccggcggggcucgcggagcuacgugccgcuggcccacagcagcagcgccuucaccaucaccgaccaggucccguucagcgugucggugagccagcuccgggcgcuggacggcggcaacaagcacuuccugcgcaaccagccccugacguucgcccugcagcugcacgacccgagcggguaccucgccgaggccgaccugagcuacaccugggacuucggcgacagcagcggcacccugaucucgcgggccccgguggugacccacaccuaccuggagccgggcccggugaccgcccaggucgugcugcaggcggccaucccgcugacgagcugcggcagcagccccgugccgggcaccaccgacgggcaccggccgaccgccgaggccccgaacaccaccgccggccaggugccgacgaccgagguggugggcaccaccccgggccaggcccccaccgcggagccgagcggcaccacgagcguccaggugccgaccaccgaggugaucucgaccgccccggugcagaugccgaccgccgagagcaccggcaugacgccggagaaggugcccgugagcgaggucauggggaccacccucgccgagaugagcaccccggaggccaccggcaugaccccggccgaggugagcaucguggugcugagcggcacgaccgcggcccaggugaccaccaccgaguggguggagaccacggcccgggagcugccgaucccggagccggagggccccgacgccucgagcaucaugagcaccgagagcaucaccggcagccugggcccgcugcuggacgggaccgccacccuccggcuggucaagcggcaggugccgcuggacugcgugcuguaccgcuacggcagcuucucggugacccuggacaucgugcagggcaucgagagcgccgagauccugcaggcggugccgagcggcgagggcgacgccuucgagcucacggucagcugccagggcgggcugccgaaggaggccugcauggagaucagcagcccgggcugccagcccccggcccagcggcugugccagccggugcugccgucgccggccugccagcuggugcugcaccagauccucaagggcggcagcggcaccuacugccugaacgugagccuggccgacaccaacagccuggcgguggugagcacccagcugaucaugccgguccccggcauccugcucaccgggcaggaggccggccugggccaggugcggcugaucgugggcauccugcuggugcugauggccguggugcucgccagccugaucuaccggcggcggcugaugaagcaggacuucucggucccgcagcugccgcacagcagcagccacuggcugcgccugccgcggaucuucugcagcugcccgaucggcgagaacagcccgcuccugucgggccagcaggug

SEQ ID NO: 49 - RNA, 711 nt, Artificial Sequence
  synthetic construct
agcggcgccgcgcgggccgccgagaucgugggcgggcacgaggcccagccgcacucgcgccccuacauggccagccugcagaugcggggcaacccgggcagccacuucugcggcggcacccucauccacccgagcuucguccugacggccgcgcacugccugcgggacaucccgcagcggcuggugaacguggugcugggggcccacaacgugcggacccaggagccgacccagcagcacuucagcguggcccaggucuuccugaacaacuacgacgccgagaacaagcucaacgacauccugcugauccagcugagcucgccggccaaccugagcgccagcgugaccagcgugcagcugccccagcaggaccagccggugccgcacggcacccagugccucgcgaugggcuggggccgggugggcgcccacgacccgccggcccaggugcugcaggagcugaacgucaccguggugacguucuucugccgcccgcacaacaucugcaccuucgugccccggcggaaggccggcaucugcuucggggacagcggcggcccgcugaucugcgacggcaucauccagggcaucgacagcuucgugaucuggggcugcgccacccggcuguucccggacuucuucacccggguggcccuguacgucgacuggauccggucgacccuccgccggguggaggcgaaggggcggccg

SEQ ID NO: 50 - RNA, 564 nt, Artificial Sequence
  synthetic construct
augaacggcgacgacgccuucgcgcggcgcccgaccgugggggcccagauccccgagaagauccagaaggccuucgacgacaucgccaaguacuucagcaaggaggagugggagaagaugaaggccucggagaagaucuucuacgucuacaugaagcggaaguacgaggccaugacgaagcugggcuucaaggcgacccucccgccguucaugugcaacaagcgggccgaggacuuccagggcaacgaccuggacaacgacccgaaccggggcaaccagguggagcggccgcagaugaccuucggccggcugcagggcaucagcccgaagaucaugcccaagaagccggccgaggaggggaacgacagcgaggaggugccggaggccagcggcccgcagaacgacggcaaggagcugugcccgccgggcaagcccaccaccagcgagaagauccacgagcgcagcggcccgaagcggggcgagcacgccuggacccaccggcugcgggagcggaagcagcuggugaucuacgaggagaucucggacccggaggaggacgacgag

SEQ ID NO: 51 - RNA, 747 nt, Artificial Sequence
  synthetic construct
augggcagcgacgugcgggaccugaacgcccuccugccggcgguccccucgcugggcggcgggggcggcugcgcccugccggugagcggcgccgcccagugggccccggugcuggacuucgccccgccgggggcgagcgccuacggcagccugggcggcccggccccgccgcccgccccgccgccgcccccgccgccgccgccgcacagcuucaucaagcaggagcccagcuggggcggcgccgagccgcacgaggagcagugccucucggccuucaccgugcacuucagcgggcaguucacgggcaccgcgggcgccugccgcuacggcccguucggcccgccgccgcccagccaggccagcagcggccaggcccggauguucccgaacgccccguaccugccgagcugccuggagucgcagccggccauccggaaccagggguacagcaccgugaccuucgacggcaccccgagcuacggccacacccccagccaccacgcggcccaguucccgaaccacagcuucaagcacgaggacccgaugggccagcagggcagccugggcgagcagcaguacucggugccgccgccggucuacgggugccacacgcccaccgacagcugcaccggcagccaggcccugcugcuccggaccccguacagcagcgacaaccuguaccagaugaccagccagcuggagugcaugaccuggaaccagaugaaccugggcgccacgcugaagggc

SEQ ID NO: 52 - RNA, 1056 nt, Artificial Sequence
  synthetic construct
auggacuuccugcggaaccucuucagccagacccugucgcugggcagccagaaggagcgccugcuggacgagcugacgcucgaggggguggcccgguacaugcagagcgagcggugccggcgggucaucugccuggugggcgcgggcaucagcaccagcgccggcaucccggacuuccggagccccucgaccggccuguacgacaaccuggagaaguaccaccugccguacccggaggccaucuucgagaucagcuacuucaagaagcacccggagccguucuucgcccuggccaaggagcucuacccgggccaguucaagcccaccaucugccacuacuucaugcgccugcugaaggacaaggggcugcugcugcggugcuacacccagaacaucgacacccucgagcggaucgccggccuggagcaggaggaccugguggaggcgcacggcacguucuacaccagccacugcgugagcgccagcugccggcacgaguacccgcugagcuggaugaaggagaagaucuucucggaggugaccccgaagugcgaggacugccagagccuggugaagccggacaucgucuucuucggcgagagccugccggcccgguucuucagcugcaugcagagcgacuuccucaagguggaccugcugcuggugaugggcaccagccugcaggugcagccguucgccucgcugaucagcaaggccccgcucagcaccccgcggcugcugaucaacaaggagaaggccggccagagcgaccccuuccuggggaugaucaugggccugggcggcggcauggacuucgacagcaagaaggcguaccgcgacguggccuggcugggcgagugcgaccaggggugccucgcccuggccgagcugcugggcuggaagaaggagcuggaggaccuggugcggcgggagcacgccagcaucgacgcccagucgggcgcgggcgucccgaacccgagcaccagcgccagcccgaagaagagcccgccgcccgccaaggacgaggcccggacgaccgagcgggagaagccgcag

SEQ ID NO: 53 - RNA, 2400 nt, Artificial Sequence
  synthetic construct
augggcagcucgaagaagcaccggggcgagaaggaggccgcggggaccacggccgccgccggcaccggcggcgccaccgagcagccgccccgccaccgggagcacaagaagcacaagcaccggagcggcgggagcggcggcagcggcggcgagcggcggaagcggagccgcgagcggggcggggagcggggcagcggccggcggggcgccgaggcggaggcccggucgagcacccacggccgcgagcggagccaggccgagccgagcgagcggcgggugaagcgggagaagcgggacgacggcuacgaggccgccgccagcagcaagaccucgagcggggacgcgagcagccugagcaucgaggagaccaacaagcuccgcgccaagcugggccugaagccgcuggaggucaacgccaucaagaaggaggccggcacgaaggaggagccggugaccgccgacgugaucaacccgauggcccugcggcagcgggaggagcugcgggagaagcucgcggccgccaaggagaagcggcugcugaaccagaagcugggcaagaucaagacccugggcgaggacgacccguggcuggacgacaccgccgccuggaucgagcggagccgccagcuccagaaggagaaggaccuggccgagaagcgggcgaagcugcuggaggagauggaccaggaguucggcgugucgacccugguggaggaggaguucgggcagcggcggcaggaccuguacagcgcccgggaccuccagggccugaccguggagcacgccaucgacagcuuccgggagggcgagacgaugauccugacccugaaggacaagggcguccugcaggaggaggaggacgugcuggugaacgugaaccucguggacaaggagcgcgccgagaagaacguggagcugcggaagaagaagcccgacuaccugccguacgccgaggacgagagcgucgacgaccuggcccagcagaagccgcggagcauccugagcaaguacgacgaggagcuggagggcgagcggccgcacucguuccggcucgagcagggcgggaccgcggacggccugcgggagcgcgagcuggaggagauccgggccaagcugcggcugcaggcccagagccugagcaccgugggcccgcggcucgccagcgaguaccugaccccggaggagauggugaccuucaagaagacgaagcggcgggugaagaagauccgcaagaaggagaaggagguggugguccgggccgacgaccugcugccccugggcgaccagacccaggacggcgacuucggcagccggcugcgggggcggggccggcgccgggugagcgagguggaggaggagaaggagccggugccgcagccgcucccgucggacgacacccggguggagaacauggacaucagcgacgaggaggagggcggcgccccgcccccgggcagcccgcaggugcuggaggaggacgaggcggagcuggagcugcagaagcagcuggagaagggccggcggcugcggcagcuccagcagcugcagcagcugcgcgacagcggggagaaggucguggagaucgugaagaagcuggagagccggcagcggggcugggaggaggacgaggacccggagcggaagggcgccaucguguucaacgccaccagcgaguucugccggacccugggcgagaucccgaccuacggccuggccggcaaccgggaggagcaggaggagcucauggacuucgagcgcgacgaggagcggucggccaacgggggcagcgagagcgacggcgaggagaacaucggcuggagcacggugaaccuggacgaggagaagcagcagcaggacuucagcgccagcucgaccaccauccuggacgaggagccgaucgugaaccggggccuggcggccgcccugcugcucugccagaacaagggccugcuggagaccaccguccagaagguggcccgggugaaggcccccaacaagagccugccgagcgccguguacugcaucgaggacaagauggcgaucgacgacaaguacagccggcgggaggaguaccgcggguucacccaggacuucaaggagaaggacggcuacaagccggacgugaagaucgaguacguggacgagacgggccggaagcugaccccgaaggaggccuuccggcagcugagccaccgguuccacggcaagggcagcggcaagaugaagaccgagcggcggaugaagaagcucgacgaggaggcccugcugaagaagaugucgagcagcgacaccccgcuggggaccgucgcccugcugcaggagaagcagaaggcccagaagaccccguacaucgugcucagcggcagcggcaagagcaugaacgccaacacgaucaccaag

SEQ ID NO: 54 - RNA, 2874 nt, Artificial Sequence
  synthetic construct
augcggacccacacgcgcggcgccccgagcguguucuucaucuaccugcucugcuucgucucggcguacaucaccgacgagaaccccgaggugaugaucccguucaccaacgccaacuacgacagccacccgaugcuguacuucagccgggccgagguggccgagcugcagcugcgggccgccagcagccacgagcacaucgcggcccggcugaccgaggccgugcacaccaugcugagcucgccgcucgaguaccugccgccgugggaccccaaggacuacagcgcccgguggaacgagaucuucgggaacaaccugggcgcccuggccauguucugcgugcuguacccggagaacaucgaggcgcgggacauggccaaggacuacauggagcgcauggccgcccagccgagcuggcuggugaaggacgccccgugggacgaggucccgcucgcccacagccuggugggcuucgcgaccgccuacgacuuccuguacaacuaccugagcaagacgcagcaggagaaguuccuggaggugaucgccaacgccagcggcuacauguacgagaccucguaccggcggggcuggggcuuccaguaccugcacaaccaccagccgaccaacugcauggcccuccugaccgggagccuggugcugaugaaccagggcuaccugcaggaggccuaccuguggaccaagcaggugcucaccaucauggagaagagccuggugcugcugcgggaggucacggacggcagccuguacgagggcguggcguacggcagcuacaccacccggagccuguuccaguacauguuccucgugcagcggcacuucaacaucaaccacuucggccaccccuggcugaagcagcacuucgccuucauguaccgcaccauccugccgggguuccagcggaccguggccaucgccgacucgaacuacaacugguucuacggcccggagagccagcugguguuccuggacaaguucgugaugcggaacggcagcggcaacuggcuggccgaccagauccggcggaaccgggucguggagggcccgggcaccccgagcaaggggcagcgcuggugcacgcuccacaccgaguuccugugguacgacggcagccugaagagcgugccgcccccggacuucggcaccccgacccugcacuacuucgaggacuggggcguggugaccuacggcucggcccugccggcggagaucaaccggagcuuccugagcuucaagagcggcaagcucgggggccgggccaucuacgacaucgugcaccggaacaaguacaaggacuggaucaagggcuggcggaacuucaacgccggccacgagcacccggaccagaacagcuucaccuucgccccgaacggcguccccuucaucacggaggcccuguacggcccgaaguacaccuucuucaacaacgugcugauguucagcccggccgugucgaagagcugcuucagcccguggguggggcaggugaccgaggacugcagcagcaaguggagcaaguacaagcacgaccuggcggccucgugccagggccggguggucgccgccgaggagaagaacggcgugguguucauccgcggcgagggcgugggcgccuacaacccgcagcugaaccugaagaacgugcagcggaaccucauccugcugcacccgcagcugcugcugcucguggaccagauccaccugggggaggagagcccccuggagaccgccgcgagcuucuuccacaacgucgacgugccguucgaggagaccgugguggacggcgugcacggcgccuucauccggcagcgggacggccuguacaagauguacuggauggacgacaccggcuacagcgagaaggccacguucgccagcgugaccuacccgcggggcuacccguacaacgggaccaacuacgucaacgugaccaugcaccugcggagcccgaucacccgcgccgccuaccuguucaucggcccgucgaucgacgugcagagcuucaccgugcacggcgacagccagcagcucgacguguucaucgcgacgagcaagcacgccuacgccaccuaccuguggaccggcgaggccaccggccagagcgccuucgcccaggugaucgcggaccggcacaagauccuguucgaccggaacagcgccaucaagucgagcaucguccccgaggugaaggacuacgccgccaucguggagcagaaccugcagcacuucaagccgguguuccagcugcuggagaagcagauccucagccgggugcggaacaccgccagcuuccggaagaccgccgagcgccugcugcgguucagcgacaagcggcagacggaggaggcgaucgaccggaucuucgccaucagccagcagcagcagcagcagucgaagagcaagaagaaccggcgggccggcaagcgcuacaaguucguggacgccgucccggacaucuucgcccagaucgaggugaacgagaagaagauccggcagaaggcccagauccuggcgcagaaggagcugccgaucgacgaggacgaggagaugaaggaccugcucgacuucgccgacgugaccuacgagaagcacaagaacgggggccugaucaagggccgguucggccaggcccggauggugaccaccacccacagccgggccccgagccugagcgccagcuacacccggcuguuccugauccugaacaucgccaucuucuucgugaugcucgcgaugcagcugacguacuuccagcgcgcccagucgcugcacggccagcggugccuguacgccgugcugcugaucgacagcugcauccuccuguggcuguacagcagcugcagccagagccagugc

SEQ ID NO: 55 - RNA, 2889 nt, Artificial Sequence
  synthetic construct
auggccaccgcggccgagacgagcgccucggagccggaggccgagagcaaggccggccccaaggccgacggggaggaggacgaggugaaggcggcccggacccgccggaagguccugagccgggccguggccgccgccaccuacaagaccaugggcccggcgugggaccagcaggaggagggcgugagcgagagcgacggcgacgaguacgccauggccagcucggccgagagcagcccgggcgaguacgagugggaguacgacgaggaggaggagaagaaccagcucgagaucgagcggcuggaggagcagcugagcaucaacguguacgacuacaacugccacguggaccugauccggcugcugcggcucgagggcgagcugaccaaggugcgcauggcccggcagaagaugagcgagaucuucccgcugaccgaggagcuguggcuggaguggcugcacgacgagaucagcauggcccaggacgggcucgaccgggagcacgucuacgaccuguucgagaaggcggugaaggacuacaucugcccgaacaucuggcuggaguacggccaguacucggugggcggcaucggccagaagggcgggcuggagaaggugcggagcguguucgagcgggcccugagcagcgugggccugcacaugacgaagggccucgcccugugggaggccuaccgggaguucgagagcgccaucgucgaggccgcgcgccuggagaaggugcacagccuguuccggcggcagcuggccaucccgcuguacgacauggaggccaccuucgccgaguacgaggaguggucggaggaccccaucccggagagcgugauccagaacuacaacaaggcccuccagcagcuggagaaguacaagccguacgaggaggcccugcugcaggcggaggccccgcggcuggccgaguaccaggccuacaucgacuucgagaugaagaucggcgacccggcccggauccagcugaucuucgagcgggcccucguggagaacugccuggugccggaccuguggauccgcuacagccaguaccuggaccggcagcugaaggugaaggaccugguccucagcgugcacaaccgggcgauccggaacugccccuggaccguggcccuguggagccgguaccugcuggccauggagcggcacggcguggaccaccaggugaucagcgugaccuucgagaaggcccugaacgccggcuucauccaggccaccgacuacgucgagaucuggcaggcguaccuggacuaccuccgccggcggguggacuucaagcaggacucgagcaaggagcuggaggagcugcgggccgccuucacccgggcccuggaguaccugaagcaggagguggaggagcgguucaacgagagcggggacccgagcugcgugaucaugcagaacugggcccgcaucgaggcccggcugugcaacaacaugcagaaggcgcgggagcucugggacagcaucaugacgcggggcaacgccaaguacgccaacauguggcuggaguacuacaaccuggagcgggcccacggcgacacccagcacugccggaaggcccugcaccgcgccgugcagugcaccagcgacuacccggagcacgugugcgagguccugcugaccauggagcggaccgagggcucgcucgaggacugggacaucgcggugcagaagaccgagacgcggcuggcccgggugaacgagcagcggaugaaggccgccgagaaggaggccgcccuggugcagcaggaggaggagaaggcggagcagcggaagcgcgcccgggccgagaagaaggcccugaagaagaagaagaagauccggggcccggagaagcggggcgccgacgaggacgacgagaaggaguggggcgacgacgaggaggagcagccgagcaagcggcggcgcguggagaacagcaucccggccgcgggggagacccagaacguggaggucgccgccggccccgccggcaagugcgccgccguggacguggagccgccgagcaagcagaaggagaaggcggccagccugaagcgggacaugccgaaggugcugcacgacagcucgaaggacagcaucaccguguucgugagcaaccucccguacagcaugcaggagccggacaccaagcugcggccccuguucgaggccugcggcgaggucgugcagauccggccgaucuucagcaaccggggcgacuuccggggguacugcuacguggaguucaaggaggagaagagcgcccugcaggcccuggagauggaccgcaagucgguggagggccggccgauguucgugagcccgugcguggacaagagcaagaacccggacuucaaggucuuccgguacagcaccagccuggagaagcacaagcucuucaucagcggccugccguucucgugcaccaaggaggagcuggaggagaucugcaaggcccacggcacggugaaggaccugcggcuggugaccaaccgggcgggcaagcccaagggccuggccuacguggaguacgagaacgagagccaggccagccaggccgugaugaagauggacgggaugaccaucaaggagaacaucaucaagguggccaucagcaacccgccgcagcggaaggucccggagaagccggagacccgcaaggccccgggcggccccaugcuccugccgcagaccuacggcgcgcggggcaagggccggacccagcugagccugcugccgcgggcccugcagcggccgagcgccgccgccccgcaggccgagaacgggccggcggccgcccccgccguggccgccccggcggccacggaggccccgaagaugucgaacgccgacuucgccaagcucuuccugcggaag

SEQ ID NO: 56 - RNA, 879 nt, Artificial Sequence
  synthetic construct
auggccgacgacgcgggcgccgccgggggcccgggcggccccggcggcccggggaugggcaaccggggcggcuuccgcggcggcuucgggagcggcauccggggccggggccggggccggggccgggggcgcggccggggccggggcgcccggggcggcaaggccgaggacaaggaguggaugccggugaccaagcuggggcggcucgucaaggacaugaagaucaagucgcuggaggagaucuaccuguucagccugccgaucaaggagagcgagaucaucgacuucuuccugggcgccagccugaaggacgaggugcucaagaucaugccggugcagaagcagacgcgggcgggccagcgcacccgguucaaggccuucguggccaucggcgacuacaacggccacgugggccugggggugaagugcagcaaggaggucgccaccgccauccggggcgccaucauccuggcgaagcugagcaucgugccggugcggcggggcuacuggggcaacaagaucggcaagccccacaccgugccgugcaaggugaccggccggugcgggucggugcugguccgccugaucccggccccgcggggcaccggcaucgugagcgccccggugccgaagaagcuccugaugauggccggcaucgacgacugcuacacgagcgcccggggcugcaccgccacccugggcaacuucgcgaaggccaccuucgacgccaucagcaagaccuacagcuaccugacccccgaccuguggaaggagacgguguucaccaagagcccguaccaggaguucaccgaccaccuggugaagacccacacccgggugucgguccagcggacccaggccccggccguggccacgacc

SEQ ID NO: 57 - RNA, 351 nt, Artificial Sequence
  synthetic construct
augagcuggcggggccgcucgaccuacuacuggccgcggccccggcgguacgugcagccgccggagaugaucgggccgaugcggccggagcaguucagcgacgaggucgagccggccacgcccgaggagggcgagccggcgacccagcggcaggacccggccgccgcccaggagggcgaggacgagggcgccagcgccggccagggcccgaagccggaggcggacagccaggagcaggggcacccgcagaccggcugcgagugcgaggacggccccgacggccaggagauggacccgccgaacccggaggaggugaagaccccggaggagggcgagaagcagagccagugc

SEQ ID NO: 58 - RNA, 1236 nt, Artificial Sequence
  synthetic construct
auggccccgguggagcacgucguggcggacgccggcgccuuccugcggcacgccgcccuccaggacaucgggaagaacaucuacaccauccgcgagguggugacggagauccgggacaaggccacccggcggcggcuggcggugcugcccuacgagcugcgguucaaggagccgcugccggaguacgugcgccuggucaccgaguucagcaagaagaccggcgacuacccgucgcucagcgccaccgacauccaggugcuggcccugaccuaccagcuggaggccgaguucgugggcgugagccaccugaagcaggagccgcagaaggugaaggugagcagcagcauccagcacccggagacgccccugcacaucucgggcuuccaccucccguacaagccgaagccgccgcaggagaccgagaagggccacagcgccugcgagccggagaaccuggaguucagcagcuucauguucuggcggaacccccugccgaacaucgaccacgagcugcaggagcugcugaucgaccggggcgaggacgucccgagcgaggaggaggaggaggaggagaacggguucgaggaccggaaggacgacagcgacgacgacggcggcggcuggaucaccccgucgaacaucaagcagauccagcaggagcucgagcagugcgacgugccggaggacgugcgggugggcugccugaccaccgacuucgccaugcagaacgugcugcugcagaugggccugcacgugcuggcggucaacgggaugcucauccgggaggcccgcagcuacauccugcggugccacggcugcuucaagaccacgagcgacaugagccggguguucugcagccacugcggcaacaagacccugaagaaggugagcgugaccgugucggacgacggcacccugcacaugcacuucagccggaacccgaaggugcugaacccccggggccugcgguacagccucccgaccccgaagggcgggaaguacgccaucaacccgcaccugaccgaggaccagcgcuucccgcagcugcggcugagccagaaggcccggcagaagacgaacgucuucgccccggacuacaucgccggcgugagccccuucguggagaacgacaucagcucgcggagcgcgacccugcaggugcgggacagcacccugggcgccggccggcgccggcucaacccgaacgccagccggaagaaguucgugaagaagcgg

SEQ ID NO: 59 - RNA, 4581 nt, Artificial Sequence
  synthetic construct
auggacgcccugugcggcagcggggagcucggcucgaaguucugggacagcaaccugagcgugcacaccgagaacccggaccugacgcccugcuuccagaacagccugcuggcgugggucccgcggaucuaccuguggguggcccucccgugcuaccugcuguaccugcgccaccacugccggggcuacaucauccugagccaccugagcaagcucaagauggugcugggcgugcugcuguggugcgugucgugggccgaccuguucuacagcuuccacggccuggugcacggccgggccccggccccggucuucuucgugaccccgcucguggugggggugaccaugcugcuggccacccugcugauccaguacgagcggcugcagggcgugcagagcagcggcguccucaucaucuucugguuccugugcguggugugcgcgaucgugcccuuccggagcaagauccugcuggccaaggccgagggcgagaucagcgacccguuccgguucaccaccuucuacauccacuucgcccuggugcugucggcccucauccuggccugcuuccgcgagaagccgccguucuucagcgcgaagaacguggacccgaacccguaccccgagacgagcgccggcuuccugagccggcuguucuucuggugguucaccaagauggccaucuacggcuaccggcacccgcuggaggagaaggaccuguggagccucaaggaggaggaccggagccagauggucgugcagcagcugcuggaggccuggcggaagcaggagaagcagaccgcccggcacaaggccucggcggccccggggaagaacgccagcggcgaggacgaggugcugcugggcgcccgcccgcggccgcggaagccgagcuuccugaaggcccuccuggccaccuucggcagcagcuuccugaucagcgcgugcuucaagcugauccaggaccugcugucguucaucaacccccagcuccugagcauccugauccgguucaucagcaacccgauggccccgagcugguggggcuuccugguggccggccugauguuccugugcagcaugaugcagagccucauccugcagcacuacuaccacuacaucuucgugaccggggugaaguuccggaccggcaucaugggcgucaucuaccggaaggcccuggugaucacgaacucggugaagcgcgccagcaccgugggcgagaucgugaaccugaugagcguggacgcccagcgguucauggaccuggcgccguuccugaaccuccuguggagcgccccgcugcagaucauccuggccaucuacuuccuguggcagaaccugggcccgagcguccucgccggcguggccuucauggugcugcugaucccccugaacggggccguggcggugaagaugcgggccuuccaggugaagcagaugaagcugaaggacagccggaucaagcugaugucggagauccucaacggcaucaagguccugaagcuguacgccugggagccgagcuuccugaagcagguggagggcauccggcagggcgagcugcagcugcuccggaccgccgccuaccugcacaccaccaccacguucaccuggaugugcagcccguuccuggugacccugaucacccuguggguguacguguacguggacccgaacaacguccuggacgccgagaaggcguucgugagcgugagccucuucaacauccugcgccugccgcugaacaugcugccgcagcugaucagcaaccucacccaggccucggugagccugaagcggauccagcaguuccugagccaggaggagcuggacccccagagcguggagcggaagaccaucagcccgggcuacgccaucacgauccacagcggcaccuucaccugggcccaggaccugccgccgacccugcacucgcucgacauccaggugccgaagggggcccuggucgccguggugggcccggugggcugcggcaagagcagccuggugagcgcgcugcugggcgagauggagaagcuggagggcaaggugcacaugaaggggagcgucgccuacgugccccagcaggccuggauccagaacugcacccuccaggagaacgugcuguucggcaaggcccugaacccgaagcgguaccagcagacccuggaggccugcgcccugcuggcggaccucgagaugcugccgggcggcgaccagacggagaucggcgagaagggcaucaaccugagcgggggccagcggcagcgggugucgcuggcccgcgccguguacagcgacgccgacaucuuccugcuggacgacccgcucagcgccguggacagccacgucgccaagcacaucuucgaccacgugaucggcccggagggcgugcuggcgggcaagacccgggugcuggugacccacggcaucagcuuccugccgcagaccgacuucaucaucgugcuggccgacgggcaggucagcgagaugggccccuacccggcccugcuccagcggaacggcucguucgccaacuuccugugcaacuacgccccggacgaggaccagggccaccuggaggacagcuggaccgcccuggagggcgcggaggacaaggaggcccugcugaucgaggacacccucagcaaccacacggaccugaccgacaacgacccggugaccuacguggugcagaagcaguucaugcggcagcugagcgcccugagcagcgacggcgaggggcagggccggccggugccgcggcgccaccugggccccucggagaaggugcaggucaccgaggccaaggccgacggcgcccugacccaggaggagaaggcggccaucggcaccguggagcucagcguguucugggacuacgccaaggccgugggccugugcacgacccuggccaucugccugcuguacguggggcagagcgccgcggccaucggcgccaacguguggcugagcgccuggaccaacgacgccauggccgacagccggcagaacaacaccagccuccggcugggcgucuacgcggcccugggcauccugcagggcuuccuggugaugcuggccgccauggccauggccgcgggcgggauccaggccgcccgggugcuccaccaggcccugcugcacaacaagauccggucgccgcagagcuucuucgacaccaccccgagcggccggauccugaacugcuucagcaaggacaucuacgugguggacgaggugcuggccccggucauccugaugcuccugaacagcuucuucaacgccaucagcacgcugguggugaucauggcgucgaccccgcuguucaccguggugauccugccgcuggccgugcucuacacccugguccagcgcuucuacgccgccaccagccggcagcugaagcggcuggagagcgugagccggagccccaucuacagccacuucucggagaccgugacgggcgccagcgugauccgggccuacaaccggagccgcgacuucgagaucaucagcgacaccaagguggacgcgaaccagcggagcugcuacccguacaucaucagcaaccgguggcugucgaucggcguggaguucgucggcaacugcguggugcuguucgccgcccucuucgccgugaucggccggagcagccugaacccggggcuggugggccugagcgugagcuacagccugcaggucaccuucgcccugaacuggaugauccggaugaugucggaccucgagagcaacaucguggccguggagcgggugaaggaguacagcaagaccgagaccgaggcgccgugggugguggagggcagccgcccgccggagggcuggcccccgcggggcgaggucgaguuccggaacuacagcgugcgguaccggccgggccuggaccuggugcugcgggaccugagccugcacgugcacgggggcgagaaggugggcaucgugggccgcaccggcgccggcaagucgagcaugacgcucugccuguuccggauccuggaggccgccaagggggagauccggaucgacggccugaacgucgccgacaucggccugcacgaccugcggagccagcucaccaucaucccgcaggacccgauccuguucagcggcacccugcggaugaaccuggacccguucggcagcuacagcgaggaggacaucuggugggcccuggagcugucgcaccuccacaccuucgugagcagccagcccgcgggccuggacuuccagugcagcgagggcggcgagaaccugagcguggggcagcggcagcuggugugccuggcccgcgcccugcuccggaagagccggauccuggugcuggacgaggccaccgccgccaucgaccuggagaccgacaaccugauccaggcgacgauccggacccaguucgacaccugcaccgugcugaccaucgcccaccggcucaacaccaucauggacuacacgcggguccuggugcuggacaagggcgugguggccgaguucgacucgccggccaaccugaucgccgcccgcggcaucuucuacggcauggcgcgggacgccgggcuggcc

SEQ ID NO: 60 - RNA, 729 nt, Artificial Sequence
  synthetic construct
augcggcacaucaucugccacggcggggugaucaccgaggagauggccgcgagccugcucgaccagcugaucgaggagguccuggccgacaaccugccgcccccgucgcacuucgagccgccgacgcugcacgagcuguacgaccucgacgugaccgccccggaggacccgaacgaggaggccgugagccagaucuuccccgagagcgugaugcuggccgugcaggagggcaucgaccuguucaccuucccgccggccccgggcagcccggagccgccccaccugagccgccagccggagcagccggagcagcgggcgcugggcccggugagcaugccgaaccuggucccggaggugaucgaccucaccugccacgaggccggcuucccgcccucggacgacgaggacgaggagggcccggugagcgagccggagccggagccggagcccgagccggagccggcccggccgacccggcggccgaagcuggugccggccauccugcggcgccccaccagcccggugagccgggagugcaacagcagcacggacucgugcgacagcgggccgagcaacaccccgccggagauccacccgguggucccccugugcccgaucaagccgguggccgugcgggugggcggccggcggcaggccguggagugcaucgaggaccugcugaacgagagcggccagccgcucgaccugagcugcaagcggccgcgcccg

SEQ ID NO: 61 - RNA, 1485 nt, Artificial Sequence
  synthetic construct
auggagcggcgcaacccgagcgagcggggcgugcccgccggguucucgggccacgcgagcgucgagagcggcggcgagacccaggagagcccggccacggugguguuccggccgccgggcaacaacaccgacggcggggccaccgccggcggcagccaggccgccgcggccgccggcgccgagccgauggagccggagagccggcccggcccgucgggcaugaacguggugcagguggccgagcuguucccggagcuccggcggauccugaccaucaacgaggacgggcagggccugaagggcgucaagcgcgagcggggcgccagcgaggcgaccgaggaggcccggaaccugaccuucagccugaugacgcggcaccggccggagugcgugaccuuccagcagaucaaggacaacugcgccaacgagcuggaccuccuggcccagaaguacagcaucgagcagcugaccaccuacuggcugcagccgggcgacgacuucgaggaggccauccggguguacgccaagguggcgcugcgcccggacugcaaguacaagaucagcaagcuggugaacauccggaacugcugcuacaucagcggcaacggggccgagguggagaucgacaccgaggaccgggucgccuuccggugcucgaugaucaacauguggcccggcgugcucggcauggacggcguggugaucaugaacgugcgguucaccggcccgaacuucagcggcacgguguuccuggccaacaccaaccugauccugcacggggucagcuucuacggcuucaacaacaccugcguggaggccuggaccgacgugcgggugcgcggcugcgccuucuacugcugcuggaagggcguggugugccggccgaagagccgggcgagcaucaagaagugccuguucgagcggugcacccugggcauccucagcgagggcaacucgcggguccggcacaacguggccagcgacugcgggugcuucaugcuggugaagagcguggccgugaucaagcacaacauggugugcggcaacugcgaggaccgcgccagccagaugcugaccugcagcgacggcaacugccaccugcugaagacgauccacgucgccagccacucgcggaaggccuggccgguguucgagcacaacauccugacccggugcagccuccaccugggcaaccggcggggcguguuccugccguaccagugcaaccugagccacaccaagauccugcuggagccggagagcaugagcaaggugaaccucaacggcguguucgacaugaccaugaagaucuggaaggugcugcgguacgacgagacccgcacccggugccggcccugcgagugcgggggcaagcacauccggaaccagccggucaugcuggacgugacggaggagcugcggccggaccaccuggugcuggcgugcacccgggccgaguucggcagcucggacgaggacaccgac

SEQ ID NO: 62 - RNA, 6093 nt, Artificial Sequence
  synthetic construct
augguggacccggucggcuucgccgaggcguggaaggcccaguuccccgacagcgagccgccgcggauggagcugcgcucggugggggacaucgagcaggagcucgagcggugcaaggccagcauccggcggcuggagcaggaggugaaccaggagcgguuccggaugaucuaccugcagacccugcuggccaaggagaagaagagcuacgaccgccagcgguggggcuuccggcgggccgcccaggcgccggacggcgccagcgagccgcgggccagcgccagccggccgcagcccgccccggccgacggcgcggacccgccgccggccgaggagccggaggcccgccccgacggcgagggcucgccggggaaggcccggccgggcacggcccggcggccgggcgccgcggccagcggcgagcgggacgaccggggcccgccggccagcguggccgcccugcgcagcaacuucgagcggauccggaagggccacgggcagcccggcgccgacgcggagaagccguucuacgugaacguggaguuccaccacgagcggggccucgucaaggugaacgacaaggaggugagcgaccggaucagcucgcugggcagccaggccaugcagauggagcggaagaagagccagcacggcgccggcagcagcgugggggacgccagccgcccgccguaccggggccggucgagcgagagcagcugcggcguggacggcgacuacgaggacgccgagcugaacccgcgguuccugaaggacaaccugaucgacgccaacggcggcagccggccgcccuggccgccgcuggaguaccagccguaccagagcaucuacgugggcgggaucauggagggcgagggcaagggcccgcuccugcggucgcagagcaccagcgagcaggagaagcgccugaccuggccgcggcggagcuacagcccccggagcuucgaggacugcggcgggggcuacaccccggacugcucgagcaacgagaaccugaccagcagcgaggaggacuucagcagcggccagucgagccgggucagcccgagcccgaccacguaccggauguuccgcgacaagagccggagcccgucgcagaacagccagcagagcuucgacagcagcagcccgcccaccccgcagugccacaagcggcaccggcacugcccggugguggugucggaggcgaccaucgugggcgugcggaagaccggccagaucuggccgaacgacgacgagggcgccuuccacggggacgccgacggcagcuucggcaccccgccgggcuacggcugcgccgccgaccgggccgaggagcagcgccggcaccaggacggccugcccuacaucgacgacagcccgagcagcagcccgcaccugucgagcaaggggcggggcagccgggacgcgcucgucagcggcgcccugaagagcaccaaggccagcgagcuggaccuggagaagggccuggagaugcggaagugggugcugucgggcauccucgccagcgaggagacguaccugagccaccuggaggcccugcugcugccgaugaagccgcucaaggccgcggccaccaccagccagccggugcugaccagccagcagaucgagaccaucuucuucaaggugcccgagcuguacgagauccacaaggagagcuacgacggccuguucccgcgggugcagcaguggucgcaccagcagcgcgugggggaccuguuccagaagcuggccagccagcucggcgucuaccgggccuucguggacaacuacggcguggccauggagauggccgagaagugcugccaggcgaacgcccaguucgccgagaucagcgagaaccugcgggcccggagcaacaaggacgccaaggacccgaccacgaagaacagccuggagacccugcuguacaagccgguggaccgggugacccggagcacccuggugcuccacgaccugcugaagcacaccccggccucgcacccggaccacccccugcugcaggacgcgcugcgcaucagccagaacuuccucagcagcaucaacgaggagaucaccccgcggcggcagagcaugacggucaagaagggcgagcaccggcagcugcugaaggacagcuucaugguggagcugguggagggcgcccggaagcugcggcacguguuccuguucaccgaccuccugcugugcaccaagcugaagaagcagucgggcgggaagacccagcaguacgacugcaagugguacaucccgcugaccgaccugagcuuccagaugguggacgagcucgaggccgugccgaacaucccgcuggucccggacgaggagcuggacgcccugaagaucaagaucagccagaucaagagcgacauccagcgcgagaagcgggccaacaagggcagcaaggccaccgagcggcugaagaagaagcugagcgagcaggagucgcuccugcugcugaugagccccagcauggcguuccgggugcacagccggaacggcaagagcuacacguuccugaucagcucggacuacgagcgggccgaguggcgcgagaacauccgggagcagcagaagaagugcuuccggagcuucagccugaccagcguggagcuccagaugcugaccaacagcugcgugaagcugcagaccgugcacagcaucccgcugaccaucaacaaggaggacgacgagucgccgggccuguacggcuuccugaacgugaucguccacagcgccaccggcuucaagcagagcagcaaggcccuccagcggccgguggccagcgacuucgagccgcaggggcugagcgaggccgcgcgguggaacucgaaggagaaccugcuggccggcccgagcgagaacgaccccaaccuguucguggcccuguacgacuucguggccagcggcgacaacacgcucagcaucaccaagggcgagaagcugcgggugcugggcuacaaccacaacggcgaguggugcgaggcccagaccaagaacgggcagggcugggugccgagcaacuacaucaccccggucaacagccuggagaagcacucgugguaccacggcccggugagccgcaacgccgcggaguaccugcugagcagcggcaucaacggcagcuuccucgugcgggagagcgagucgagcccgggccagcggagcaucagccugcgguacgaggggcggguguaccacuaccggaucaacaccgccagcgacggcaagcuguacgugagcucggagagccgcuucaacacccuggccgagcuggugcaccaccacagcacggucgccgacggccugaucaccacccuccacuacccggcccccaagcggaacaagccgaccguguacggcgugagcccgaacuacgacaagugggagauggagcggaccgacaucaccaugaagcacaagcugggcggcgggcaguacggcgagguguacgagggcguguggaagaaguacagccugacgguggccgucaagacccugaaggaggacaccauggagguggaggaguuccugaaggaggcggccgugaugaaggagaucaagcacccgaaccuggugcagcuccugggcgugugcacccgggagccgccguucuacaucaucaccgaguucaugaccuacggcaaccugcuggacuaccugcgggagugcaaccggcaggaggugaacgccgucgugcugcucuacauggccacgcagaucagcucggccauggaguaccuggagaagaagaacuucauccaccgcgaccuggccgcgcggaacugccuggugggcgagaaccaccuggugaagguggccgacuucgggcugagccggcucaugaccggcgacaccuacaccgcccacgccggcgccaaguuccccaucaaguggaccgccccggagagccuggcguacaacaaguucagcaucaagagcgacgugugggccuucggcguccugcugugggagaucgccaccuacggcaugagcccguacccgggcaucgaccugucgcagguguacgagcugcucgagaaggacuaccggauggagcggccggaggggugcccggagaagguguacgagcugaugcgggccugcuggcaguggaaccccagcgaccgcccgagcuucgccgagauccaccaggccuucgagacgauguuccaggagagcagcaucagcgacgagguggagaaggagcugggcaagcagggcgugcggggcgcggugucgacccugcugcaggccccggagcugccgaccaagacccggaccagccggcgggccgccgagcaccgggacaccacggacgucccggagaugccgcacagcaagggccagggcgagagcgacccccucgaccacgagccggccgugagcccgcugcugccgcgcaaggagcgggggccgccggagggcggccugaacgaggacgagcggcugcugcccaaggacaagaagaccaaccucuucagcgcccugaucaagaagaagaagaagaccgcgccgaccccgccgaagcggucgagcagcuuccgggagauggacggccagccggagcggcgcggcgccggcgaggaggaggggcgggacaucagcaacggcgcccuggccuucaccccgcuggacaccgccgaccccgccaagagcccgaagccgagcaacggcgcgggcgugccgaacggcgcccugcgggagucgggcgggagcggcuuccggagcccgcaccuguggaagaagagcagcacgcucaccagcucgcggcuggccaccggcgaggaggagggcggcggcagcagcagcaagcgguuccugcgcagcugcagcgccucgugcgugccgcacggggccaaggacaccgaguggcggagcgugacccugccccgggaccugcagagcaccggccggcaguucgacagcagcacguucggcggccacaagagcgagaagccggcccugccgcggaagcgggcgggcgagaaccgcucggaccaggugacccggggcaccgucaccccgccgccgcggcucgugaagaagaacgaggaggccgccgacgagguguucaaggacaucauggagagcagccccgggagcagcccgccgaaccugaccccgaagccgcugcggcggcaggugaccguggccccggccagcggccugccccacaaggaggaggccgagaagggcucggcgcugggcacgccggccgccgccgagccggugaccccgaccagcaaggccggcagcggcgccccgggcgggaccagcaagggcccggcggaggagagccggguccgccggcacaagcacagcucggagagccccggccgggacaagggcaagcugagccggcucaagccggccccgccgccgccgcccgccgccagcgccggcaaggccgggggcaagccgagccagagcccgucgcaggaggcggccggcgaggccgugcugggcgccaagaccaaggccaccagccugguggacgccgugaacagcgacgcggccaagccgagccagccgggcgagggccugaagaagccggugcugcccgccacgccgaagccgcagagcgccaagccgagcgggaccccgaucucgccggcccccgugccgagcacccugccgagcgccagcagcgcgcucgccggcgaccagccgagcucgaccgccuucaucccgcugaucagcacccgggucagccugcggaagacccgccagccgcccgagcggaucgccagcggcgccaucacgaagggcguggugcuggacagcaccgaggcccugugccuggcgaucagccggaacucggagcagauggccagccacagcgccgugcucgaggccggcaagaaccuguacaccuucugcgugagcuacguggacagcauccagcagaugcggaacaaguucgccuuccgggaggccaucaacaagcuggagaacaaccugcgggagcugcagaucugcccggcgaccgccggcagcgggccggccgccacccaggacuucucgaagcugcucagcagcgucaaggagaucagcgacaucgugcagcgc

SEQ ID NO: 63 - RNA, 1527 nt, Artificial Sequence; identical to SEQ ID NO: 33
  synthetic construct

SEQ ID NO: 64 - RNA, 768 nt, Artificial Sequence
  synthetic construct
auggcccaccggccgcccagcccggcgcuggccucggugcuccuggcccugcugcugagcggcgccgcccgcgccgcggagaucgucgggggccacgaggcccagccgcacagccggccguacauggccagccugcagaugcggggcaacccgggcagccacuucugcggcggcacccucauccacccgagcuucgugcugacggccccgcacugccugcgggacaucccccagcggcuggugaacguggugcugggggcccacaacgugcggacccaggagccgacccagcagcacuucucggucgcccagguguuccugaacaacuacgacgcggagaacaagcucaacgacauccugcugauccagcugagcagcccggccaaccugagcgccagcgugaccagcgugcagcugccgcagcaggaccagccggugccccacggcacccagugccucgccaugggcuggggccgcgugggcgcccacgacccgccggcccagguccugcaggagcugaacgugaccguggugacguucuucugccggccgcacaacaucugcaccuucgugccgcggcggaaggcgggcaucugcuucggggacucgggcggcccgcugaucugcgacggcaucauccagggcaucgacagcuucgugaucuggggcugcgccacccggcuguuccccgacuucuucacccgggucgcccuguacguggacuggauccgcagcacccuccggcggguggaggccaaggggcggccg

SEQ ID NO: 65 - RNA, 1767 nt, Artificial Sequence
  synthetic construct
augaucgccagccaguuccugucggcgcucacccuggugcugcugaucaaggagagcggcgccuggagcuacaacacgagcaccgaggccaugaccuacgacgaggccagcgccuacugccagcagcgguacacccaccuggucgccauccagaacaaggaggagaucgaguaccugaacagcauccucucguacagcccgagcuacuacuggaucgggauccgcaaggugaacaacgugugggugugggugggcacccagaagccccugaccgaggaggcgaagaacugggccccgggcgagccgaacaaccggcagaaggacgaggacugcguggagaucuacaucaagcgggagaaggacgucggcauguggaacgacgagcggugcagcaagaagaagcuggcccugugcuacacggccgccugcaccaacaccagcugcagcggccacggcgagugcguggagaccaucaacaacuacaccugcaagugcgacccgggguucucgggccugaagugcgagcagaucgugaacugcaccgcccuggagagcccggagcacggcagccucgugugcagccacccgcugggcaacuucagcuacaacagcucgugcagcaucagcugcgaccggggcuaccugcccagcagcauggagacgaugcagugcaugagcucgggcgaguggagcgcgccgaucccggccugcaacgugguggagugcgacgccgucaccaacccggccaacggguucguggagugcuuccagaacccgggcagcuucccguggaacaccaccugcaccuucgacugcgaggagggcuucgagcugaugggcgcccagagccugcagugcaccagcagcggcaacugggacaacgagaagcccacgugcaaggccgugaccugccgggcggugcgccagccgcagaacggcucggugcggugcagccacagcccggccggggaguucaccuucaagagcagcugcaacuucaccugcgaggagggcuucaugcugcagggcccggcccagguggagugcaccacccagggccaguggacgcagcagaucccggucugcgaggccuuccagugcaccgcccucagcaacccggagcggggcuacaugaacugccugcccucggccagcggcagcuuccgguacgggagcagcugcgaguucagcugcgagcagggcuucgugcugaagggcucgaagcggcugcagugcggcccgaccggcgagugggacaacgagaagccgaccugcgaggcggugcggugcgacgccgugcaccagccgccgaagggccuggugcgcugcgcccacagcccgaucggggaguucaccuacaagagcagcugcgccuucagcugcgaggagggcuucgagcuguacggcagcacccagcucgagugcacgucgcagggccaguggaccgaggaggugcccagcugccaggucgugaagugcagcagccuggccgugccgggcaagaucaacaugagcugcagcggcgagccgguguucgggaccgugugcaaguucgccugcccggagggcuggacccugaacggcucggcggcccggaccugcggcgccaccggccacuggagcggccugcugccgacgugcgaggccccgaccgagagcaaccuguucauccccguggccgucauggugaccgccuucagcggguucaucuaccaccucgcgggcaaggagaucaagaagcggcaggagauccaggagaaguacgag

SEQ ID NO: 66 - RNA, 1557 nt, Artificial Sequence
  synthetic construct
augagcccgcugugguggggcuuccuccugucgugccuggggugcaagauccugcccggcgcccagggccaguucccgcgggugugcaugaccgucgacagccuggugaacaaggagugcugcccgcgccugggcgcggagagcgccaacgugugcggcagccagcagggccgggggcagugcacggaggugcgggccgacacccggccguggagcggcccguacauccuccggaaccaggacgaccgggagcuguggccgcgcaaguucuuccaccggaccugcaagugcaccggcaacuucgccggcuacaacugcggcgacugcaaguucggcuggaccgggcccaacugcgagcggaagaagccgccggugauccggcagaacauccacagccugucgccgcaggagcgggagcaguuccugggcgcccuggaccuggccaagaagcgggugcacccggacuacgucaucaccacgcagcacuggcucggccugcugggcccgaacggcacccagccccaguucgcgaacugcagcguguacgacuucuucguguggcugcacuacuacagcgugcgcgacacccugcugggcccggggcggccguaccgggccaucgacuucagccaccagggcccggccuucgugaccuggcaccgguaccaccuccugugccuggagcgggaccugcagcggcugaucggcaacgagagcuucgcccugccguacuggaacuucgccaccggccgcaacgagugcgacgugugcaccgaccagcucuucggcgccgcgcggccggacgaccccacgcugaucagccggaacucgcgguucagcagcugggagaccgucugcgacagccuggacgacuacaaccaccuggugacccugugcaacggcaccuacgaggggcugcuccggcggaaccagaugggccgcaacagcaugaagcugccgacccugaaggacauccgggacugccugagccugcagaaguucgacaacccgccguucuuccagaacucgaccuucagcuuccggaacgcccuggagggcuucgacaaggccgacggcacgcucgacagccaggugaugagccugcacaaccuggugcacagcuuccugaacggcaccaacgcccugccgcacagcgccgccaacgacccgaucuucguggugcugcacucguucaccgacgcgaucuucgacgaguggaugaagcgguucaacccgcccgccgacgccuggccgcaggagcucgccccgaucggccacaaccggauguacaacauggucccguucuucccgcccgugaccaacgaggagcuguuccugaccagcgaccagcugggguacagcuacgccaucgaccugccggugagcguggaggagaccccgggcuggccgacgacccugcucguggugaugggcacccuggucgcccuggugggccuguucgugcugcuggcguuccuccaguaccggcgccugcggaagggcuacaccccgcugauggagacccaccugagcagcaagcgguacaccgaggaggcc

SEQ ID NO: 67 - RNA, 600 nt, Artificial Sequence
  synthetic construct
augggcaacagcaagucgggggcccugagcaaggagauccucgaggagcugcagcugaacaccaaguucagcgaggaggagcugugcagcugguaccagagcuuccugaaggacugcccgacgggccggaucacccagcagcaguuccagagcaucuacgcgaaguucuuccccgacaccgacccgaaggccuacgcccagcacguguuccgcucguucgacagcaaccuggacggcacccucgacuucaaggaguacgucaucgcccugcacaugaccaccgccggcaagacgaaccagaagcuggagugggccuucagccuguacgacguggacggcaacggcaccaucagcaagaacgaggugcuggagaucgugauggcgaucuucaagaugaucaccccggaggacgugaagcugcucccggacgacgagaacaccccggagaagcgggccgagaagaucuggaaguacuucgggaagaacgacgacgacaagcugaccgagaaggaguucaucgagggcacccuggccaacaaggagauccugcggcugauccaguucgagccgcagaaggugaaggagaagaugaagaacgcc

SEQ ID NO: 68 - RNA, 3396 nt, Artificial Sequence
  synthetic construct
augccgcgggccccgcgcugccgggcggugcggagccugcuccggucgcacuaccgggagguccugccccuggccaccuucgugcggcgccugggcccgcagggguggcggcuggugcagcggggcgacccggccgccuuccgggcccugguggcccagugccucgugugcgugccgugggacgcgcggccgcccccggccgccccgagcuuccggcaggucagcugccugaaggagcugguggcccgcgugcugcagcggcugugcgagcggggcgccaagaacgugcuggccuucggcuucgcgcuccuggacggcgcccggggcgggccgccggaggccuucacgaccagcgugcggagcuaccugccgaacaccgugaccgacgcccugcggggcagcggcgccuggggccugcugcuccgccgggucggcgacgacgugcuggugcaccugcuggcccggugcgcgcuguucgugcugguggcccccucgugcgccuaccaggugugcggcccgccgcucuaccagcugggggccgccacccaggcccggccgccgccgcacgcgagcggcccccggcggcgccugggcugcgagcgggccuggaaccacagcguccgggaggccggcgugccgcugggccugccggccccgggcgcccggcggcggggcgggagcgccagccgcagccugccgcucccgaagcggccccggcggggcgcggccccggagccggagcggaccccggugggccagggcucgugggcccacccgggccggacgcgcgggccgagcgaccggggcuucugcguggugagccccgcccggccggccgaggaggccaccagccuggagggcgcgcugagcggcacccggcacagccacccgucggugggccggcagcaccacgccggcccgccgagcaccagccggccgccccgcccgugggacaccccgugcccgccggucuacgccgagaccaagcacuuccuguacagcagcggggacaaggagcagcugcggccgagcuuccugcucucgagccugcggcccagccugacgggcgcccggcggcugguggagaccaucuuccugggcagccggccguggaugccgggcaccccgcgccggcugccgcggcucccgcagcgguacuggcagaugcggccccuguuccuggagcugcugggcaaccacgcccagugcccguacggcgugcugcucaagacccacugcccgcugcgggccgcggugaccccggccgccggggugugcgcccgcgagaagccgcagggcagcguggccgccccggaggaggaggacaccgacccccggcggcugguccagcugcugcggcagcacagcucgccguggcagguguacggcuucgugcgggcgugccugcggcgccucgugccgccgggccuguggggcagccggcacaacgagcggcgguuccugcggaacacgaagaaguucaucagccugggcaagcacgccaagcugagccugcaggagcucaccuggaagaugagcgugcgggacugcgccuggcugcgccggagcccgggggugggcugcgucccggccgccgagcaccggcugcgggaggagauccuggccaaguuccugcacuggcugaugucgguguacgugguggagcuccugcggagcuucuucuacgugaccgagaccaccuuccagaagaaccggcuguucuucuaccgcaagagcguguggagcaagcugcagagcaucggcauccggcagcaccugaagcggguccagcugcgggagcucagcgaggcggaggugcggcagcaccgggaggcccgccccgcccugcugaccucgcggcugcgguucaucccgaagccggacggccugcggccgaucgugaacauggacuacguggugggcgcccggacguuccggcgcgagaagcgggccgagcggcugaccagccgggugaaggcccucuucagcguccugaacuacgagcgggcgcggcgcccgggccugcugggggccagcgugcugggccuggacgacauccaccgggccuggcggaccuucgugcuccgggugcgggcccaggacccgcccccggagcuguacuucgugaagguggacgucaccggcgccuacgacaccaucccgcaggaccggcugaccgaggugaucgccagcaucaucaagccgcagaacacguacugcgugcgccgguacgcgguggugcagaaggccgcccacggccacgugcggaaggccuucaagagccacgucucgacccugaccgaccugcagccguacaugcggcaguucguggcccaccugcaggagaccagcccgcuccgggacgccguggugaucgagcagagcagcagccugaacgaggcgagcucgggccuguucgacguguuccugcgguucaugugccaccacgccgugcgcauccggggcaagagcuacguccagugccaggggaucccccagggcagcauccugagcacccugcucugcagccugugcuacggcgacauggagaacaagcuguucgccggcauccggcgggacggccugcugcugcggcucguggacgacuuccugcuggugaccccgcaccugacgcacgccaagaccuuccugcggacccuggugcgcggcgugccggaguacgggugcguggucaaccuccggaagaccguggugaacuucccgguggaggacgaggcccugggcggcaccgccuucgugcagaugccggcgcacggccuguucccguggugcggccugcugcuggacacccggacgcucgaggugcagagcgacuacucgagcuacgcccggaccagcauccgggccagccugaccuucaaccggggcuucaaggccgggcgcaacaugcggcggaagcuguucggcguccugcggcugaagugccacagccuguuccucgaccugcaggugaacagccugcagaccgugugcaccaacaucuacaagauccugcugcugcaggccuaccgguuccacgccugcgugcuccagcugcccuuccaccagcagguguggaagaacccgaccuucuuccugcgggugaucucggacacggcgagccugugcuacagcauccugaaggccaagaacgccggcaugagccugggcgccaagggcgccgccggcccgcucccgagcgaggcgguccaguggcugugccaccaggccuuccugcugaagcugacccgccaccgggugaccuacgugccgcugcucgggagccugcggaccgcccagacccagcugucgcggaagcugccgggcaccacgcugaccgcccuggaggccgccgcgaaccccgcccucccgagcgacuucaagaccauccuggac

SEQ ID NO: 69 - RNA, 354 nt, Artificial Sequence
  synthetic construct
augccgcgggaggacgcccacuucaucuacggcuaccccaagaaggggcacggccacagcuacaccacggcggaggaggccgccggcaucggcauccugaccgugauccucggcguccugcugcugaucggcugcugguacugccgccggcggaacggguaccgggcccugauggacaagucgcugcacgugggcacccagugcgcccucacccggcggugcccgcaggagggcuucgaccaccgcgacagcaaggugagccugcaggagaagaacugcgagccgguggugccgaacgccccgccggcguacgagaagcugagcgccgagcagagcccgccgcccuacagcccg

SEQ ID NO: 70 - RNA, 942 nt, Artificial Sequence
  synthetic construct
augccgcuggagcagcggagccagcacugcaagcccgaggagggccucgaggcccgcggggaggcgcugggccuggugggcgcccaggccccggccaccgaggagcaggaggccgccucgagcagcagcacgcuggucgaggugacccugggcgaggugccggcggccgagagcccggacccgccgcagagcccccagggcgccucgagccugccgaccaccaugaacuacccgcucuggagccagagcuacgaggacagcagcaaccaggaggaggagggcccgucgaccuucccggaccuggagagcgaguuccaggccgcccugagccggaagguggccgagcuggugcacuuccugcugcucaaguaccgggcgcgggagccggugaccaaggccgagaugcuggggagcgucgugggcaacuggcaguacuucuuccccgugaucuucagcaaggccagcucgagccugcagcugguguucggcaucgagcugauggagguggacccgaucggccaccuguacaucuucgccacgugccucggccugagcuacgacggccugcugggggacaaccagaucaugccgaaggccggccugcugaucaucgugcucgccaucaucgcgcgggagggcgacugcgccccggaggagaagaucugggaggagcugagcguccuggagguguucgagggccgggaggacagcauccugggcgacccgaagaagcugcugacccagcacuucgugcaggagaacuaccucgaguaccgccaggugccgggcagcgaccccgccugcuacgaguuccugugggggccgcgggcccugguggagaccucguacgugaagguccugcaccacauggugaagaucagcggcggcccgcacaucagcuacccgccgcugcacgagugggugcugcgggagggcgaggag

SEQ ID NO: 71 - RNA, 927 nt, Artificial Sequence
  synthetic construct
augagccuggagcagcggucgcuccacugcaagccggaggaggcccuggaggcgcagcaggaggcccugggccuggugugcguccaggccgccaccagcagcagcagcccccuggugcuggggacgcucgaggaggugccgaccgccggcagcaccgacccgccgcagucgccgcagggcgccagcgcguucccgaccaccaucaacuucacccgccagcggcagcccagcgagggcagcagcagccgggaggaggagggcccgucgacgagcugcauccuggagagccuguuccgggccgugaucaccaagaagguggccgaccuggugggcuuccugcugcucaaguaccgggcccgggagccggucaccaaggccgagaugcuggagagcgugaucaagaacuacaagcacugcuucccggagaucuucgggaaggccagcgagagccugcagcugguguucggcaucgacgugaaggaggcggacccgaccggccacucguacgugcuggugaccugccugggccucagcuacgacggccugcugggcgacaaccagaucaugccgaagaccggguuccugaucaucguccuggugaugaucgccauggagggcggccacgcccccgaggaggagaucugggaggagcugagcgugauggagguguacgacggccgcgagcacagcgccuacggcgagccgcggaagcuccugacgcaggaccuggugcaggagaaguaccuggaguaccggcaggugccggacagcgacccggcccgguacgaguuccuguggggcccgcgggcccuggcggagaccagcuacgucaaggugcucgaguacgugaucaaggugucggcccgggugcgcuucuucuucccgagccugcgggaggccgcccugcgggaggaggaggagggggug

SEQ ID NO: 72 - RNA, 1587 nt, Artificial Sequence
  synthetic construct
augcugcucgccgugcuguacugccugcuguggagcuuccagaccucggcgggccacuucccgcgggccugcgucagcagcaagaaccugauggagaaggagugcugcccgcccuggagcggggaccgcagcccgugcggccagcugagcggccggggcucgugccagaacauccuccugagcaacgccccgcugggcccgcaguucccguucacgggcguggacgaccgggagagcuggcccagcguguucuacaaccggaccugccagugcagcgggaacuucaugggcuucaacugcggcaacugcaaguucggcuucuggggcccgaacugcaccgagcggcggcugcuggugcgccggaacaucuucgaccugagcgccccggagaaggacaaguucuucgccuaccucacccuggccaagcacaccaucucgagcgacuacgugaucccgaucggcaccuacgggcagaugaagaacggcagcacgccgauguucaacgacaucaacaucuacgaccuguucguguggaugcacuacuacgucagcauggacgcgcugcugggcggcagcgagaucuggcgggacaucgacuucgcccacgaggccccggccuuccugcccuggcaccggcucuuccugcugcggugggagcaggagauccagaagcugaccggcgacgagaacuucaccaucccguacugggacuggcgggacgccgagaagugcgacaucugcaccgacgaguacaugggcgggcagcacccgaccaacccgaaccugcugagcccggccucguucuucagcagcuggcagaucgugugcagccgccucgaggaguacaacagccaccagagccugugcaacggcaccccggagggcccccugcggcggaacccgggcaaccacgacaagucgcggacgccgcggcugccgagcagcgcggacguggaguucugccugagccugacccaguacgagagcggcagcauggacaaggccgccaacuucucguuccggaacacccucgagggcuucgccagcccgcugaccgggaucgccgacgccagccagagcagcaugcacaacgcgcugcacaucuacaugaacggcaccaugagccaggugcagggcucggccaacgacccgaucuuccugcugcaccacgccuucguggacagcaucuucgagcaguggcugcagcgccaccggccccuccaggagguguacccggaggccaacgccccgaucggccacaaccgggagagcuacauggucccguucaucccgcuguaccggaacggcgacuucuucaucagcagcaaggaccugggcuacgacuacagcuaccugcaggacucggacccggacagcuuccaggacuacaucaagagcuaccuggagcaggccagccggaucuggagcuggcugcucggggcggccauggugggcgccgugcugaccgcccugcuggccggccuggugagccugcucugccggcacaagcgcaagcagcugcccgaggagaagcagccgcugcugauggagaaggaggacuaccacucgcuguaccagagccaccug

SEQ ID NO: 73 - RNA, 294 nt, Artificial Sequence
  synthetic construct
augcacggcgacaccccgacgcugcacgaguacaugcucgaccugcagcccgagaccaccgaccuguacugcuacgagcagcugaacgacagcucggaggaggaggacgagaucgacgggccggccggccaggcggagccggaccgggcccacuacaacaucgugaccuucugcugcaagugcgacagcacccugcgccugugcguccagagcacccacguggacauccggacgcucgaggaccugcugaugggcacccugggcaucgugugcccgaucugcagccagaagccg

SEQ ID NO: 74 - RNA, 1683 nt, Artificial Sequence
  synthetic construct
auggagagccggggccgccggugcccggagaugaucucggugcuggggcccaucagcggccacguccucaaggccguguucagccggggcgacaccccggugcugccgcacgagacgcggcugcugcagaccggcauccacgugcgggugagccagccgagccugauccuggugagccaguacaccccggacucgaccccgugccaccggggcgacaaccagcuccagguccagcacaccuacuucaccggcagcgagguggagaacgugagcgugaacgugcacaaccccacggggcgcagcaucugcccgagccaggagccgaugagcaucuacguguacgcgcugccgcugaagaugcugaacaucccgucgaucaacguccaccacuacccgagcgccgccgagcggaagcaccggcaccugcccguggccgacgccgugauccacgccagcggcaagcagauguggcaggcgcggcugaccgugagcggccucgccuggacccggcagcagaaccaguggaaggagccggacguguacuacaccagcgccuucguguucccgaccaaggacgucgcccugcggcacguggugugcgcccacgagcuggugugcagcauggagaacacccgcgccacgaagaugcaggugaucggcgaccaguacgugaaggucuaccuggagucguucugcgaggacgugccgagcggcaagcuguucaugcacgugacccugggcagcgacguggaggaggaccucaccaugacccggaacccgcagccguucaugcggccccacgagcggaacggguucaccgugcugugcccgaagaacaugaucaucaagccgggcaagaucagccacaucaugcuggacguggcguucaccagccacgagcacuucggccugcugugcccgaagagcaucccgggccugucgaucagcggcaaccuccugaugaacggccagcagaucuuccuggagguccaggccauccgggagacgguggagcugcggcaguacgacccgguggccgcccuguucuucuucgacaucgaccugcuccugcagcgcgggccccaguacagcgagcacccgaccuucaccagccaguaccggauccagggcaagcuggaguaccggcacaccugggaccggcacgacgagggcgccgcccagggcgacgacgacguguggaccagcggcagcgacucggacgaggagcuggugaccacggagcggaagaccccgcgggugaccggcgggggcgcgauggccggcgccagcaccagcgccggccgcaagcggaagagcgccagcagcgccaccgcgugcaccucgggcgucaugacgcggggccggcugaaggccgagagcaccguggccccggaggaggacaccgacgaggacagcgacaacgagauccacaacccggccguguucaccuggccgcccuggcaggccgggauccuggcccggaaccucgugccgaugguggcgaccgugcagggccagaaccugaaguaccaggaguucuucugggacgccaacgacaucuaccggaucuucgccgagcuggagggcgucuggcagccggccgcccagccgaagcgccggcggcaccggcaggacgcccugccgggcccgugcaucgcgagcacccccaagaagcaccggggc

SEQ ID NO: 75 - RNA, 1074 nt, Artificial Sequence
  synthetic construct
augcagaugaacagccagccgucgcccaacagcgcccggagccagagcgagcgcccgguguucccgugcugguggcugcaguuccggaacagcaagccgugcagcgacuacugccucucgcugaucgucaaccugcuggaggacuggggcccgugcgcggagcacggggagcaccacauccggaucccgcggacccccagccgggugacgggcggcguguuccugguggacaagaacccgcacaacaccgccgagagccggcuggugguggacuucagccaguucagccgcggcaacuaccgggucagcuggccgaaguucgccgugccgaaccuccagucgcugaccaaccugcugagcagcaaccugagcuggcugagccucgacgugagcgccgccuucuaccaccugccgcugcacccggccgcgaugccccaccugcuggugggcucgagcggccugagccgguacguggcccggcucagcagcaacagccggauccugaacaaccagcacgggaccaugccggaccugcacgacuacugcucgcggaaccuguacgugagccugcugcuccuguaccagaccuucggccgcaagcugcaccuguacagccacccgaucauccugggcuuccggaagaucccgaugggcgucggccugagcccguuccuccuggcccaguucaccagcgccaucugcagcguggugcggcgggccuucccgcacugccuggccuucucguacauggacgacguggugcugggcgcgaagagcgugcagcaccuggagagccuguucacggccgucaccaacuuccuccugagccuggggauccaccugaaccccaacaagaccaagcgguggggcuacagccugaacuucaugggcuacgugaucggcugcuacggcagccugccgcaggagcacaucauccagaagaucaaggagugcuuccggaagcucccgaucaaccgcccgaucgacuggaaggugugccagcggaucgugggccugcugggguucgccgccccguucacccagugcggcuacccggcccugaugccccuguac

SEQ ID NO: 76 - RNA, 13 nt, Artificial Sequence
  synthetic construct
gccgccaccaugg

SEQ ID NO: 77 - RNA, 15 nt, Unknown
  synthetic construct
nccancccnnucncc

SEQ ID NO: 78 - DNA, 45 bp, Xenopus sp.
gcttgttctttttgcagaagctcagaataaacgctcaactttggc

SEQ ID NO: 79 - DNA, 157 bp, Xenopus sp.
gactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattcta

SEQ ID NO: 80 - DNA, 45 bp, Homo sapiens
tagcccgctgggcctcccaacgggccctcctcccctccttgcacc

(19) United States
(12) Patent Application Publication
(10) Pub. No.: US 2008/0171711 A1
(43) Pub. Date: Jul. 17, 2008

(54) Mrna Mixture For Vaccinating Against Tumoral Diseases

(75) Inventors: Ingmar Hoerr, Tubingen (DE); Steve Pascolo, Tubingen (DE)

Correspondence Address: CONNOLLY BOVE LODGE & HUTZ LLP, 1875 EYE STREET, N.W., SUITE 1100, WASHINGTON, DC, 20036, US

(73) Assignee: CureVac GmbH

(21) Appl. No.: 11/632,802

(22) PCT Filed: Jul. 20, 2005
(86) PCT No.: PCT/EP2005/007930

§ 371 Date: Nov. 13, 2007

(30) Foreign Application Priority Data
Jul. 21, 2004 (DE) 10 2004 035 227.5

(51) Int. Cl. A61K 31/7105; A61P 35/00; A61P 37/04

(52) U.S. Cl. 514/44

(57) ABSTRACT

The present invention relates to a mixture which contains mRNA for vaccination, wherein at least one mRNA contains a domain which codes for at least one antigen from a tumor and at least one further mRNA contains a domain which codes for at least one immunogenic protein (polypeptide). The invention furthermore relates to a pharmaceutical composition which contains an mRNA mixture according to the invention, and to the use for the treatment of tumor diseases.

The present invention relates to a mixture which contains mRNA for vaccination, wherein at least one mRNA contains a domain which codes for at least one antigen from a tumor and at least one further mRNA contains a domain which codes for at least one immunogenic protein. The invention furthermore relates to a pharmaceutical composition which contains an mRNA mixture according to the invention, and the use for the treatment of tumor diseases.

Methods of molecular medicine, such as gene therapy and genetic vaccination, play a major part in the treatment and prevention of numerous diseases. These methods are based on the introduction of nucleic acids into the patient's cells or tissue, followed by processing of the information coded by the introduced nucleic acids, i.e. expression of the desired polypeptides or proteins. Nucleic acids which may be considered for introduction in these methods are both DNA and RNA.

Hitherto conventional methods of gene therapy and genetic vaccination are based on the use of DNA in order to incorporate the required genetic information into the cell. Various methods have been developed in this connection for introducing DNA into cells, such as for example calcium phosphate transfection, polyprene transfection, protoplast fusion, electroporation, microinjection and lipofection, lipofection having in particular proven to be a suitable method. DNA viruses may likewise be used as a DNA vehicle. Because of their infectious properties, such viruses achieve a very high transfection rate. The viruses used are genetically modified in this method in such a manner that no functional infectious particles are formed in the transfected cell. Despite these precautions, however, it is not possible to rule out the risk of uncontrolled propagation of the introduced gene-therapeutically effective and viral genes, for example due to possible recombination events.

As mentioned, not only DNA but also RNA may be considered as a usable nucleic acid in gene therapy. And although it is known in the prior art that the instability of mRNA or of RNA in general may be a problem in the application of medical methods based on RNA expression systems, RNA expression systems have considerable advantages over DNA expression systems in gene therapy and in genetic vaccination. These include, inter alia, that RNA introduced into a cell is not integrated into the genome, whereas when using DNA (for example as a DNA vehicle which is derived from DNA viruses) which is introduced into a cell, this DNA is integrated to a certain extent into the genome. This entails a risk of the DNA being inserted into an intact gene of the host cell's genome, with the consequence that this gene may be mutated and thus completely or partially inactivated or give rise to misinformation. In other words, synthesis of a gene product which is vital to the cell may be completely suppressed or alternatively a modified or incorrect gene product is expressed. One particular risk is if the DNA is integrated into a gene which is involved in the regulation of cell growth. In this case, the host cell may become degenerate and lead to cancer or tumor formation. Furthermore, if the DNA introduced into the cell is to be expressed, it is necessary for the corresponding DNA vehicle to contain a strong promoter, such as the viral CMV promoter. The integration of such promoters into the genome of the treated cell may result in unwanted changes in the regulation of gene expression in the cell. In contrast, when RNA is used as a vaccine, no viral sequences, such as promoters etc., are necessary for active transcription.

Another risk of using DNA as a vaccine (or gene therapy agent) is the induction of pathogenic anti-DNA antibodies in the patient into whom the foreign DNA has been introduced, so bringing about a (possibly fatal) immune response. In contrast, no anti-RNA antibodies have yet been detected. The reason for this will be the fact that RNA is substantially more straightforwardly degraded in vivo, i.e. in the patient's body. In comparison with DNA, RNA has a relatively short half-life in the bloodstream.

Despite the above-mentioned numerous advantages of using RNA in comparison with DNA in methods of molecular genetics, the already mentioned instability of RNA is a problem. The instability of RNA is in particular due to RNA-degrading enzymes, “RNAases” (ribonucleases), even the slightest contamination with ribonucleases being sufficient completely to degrade RNA in solution. There are also many further processes which destabilize RNA. Many of these processes are as yet unknown, but interaction between the RNA and proteins often appears to play a crucial role. On the other hand, numerous phenomena which stabilize RNA are also known.

Some measures for increasing the stability of RNA, so enabling the use thereof as a gene therapy agent or RNA vaccine, have been proposed in this connection in the prior art.

In order to solve the problem of ex vivo RNA stability, EP-A-1083232 proposes a method for introducing RNA, in particular mRNA, into cells and organisms, in which the RNA assumes the form of a complex with a cationic peptide or protein.

WO 99/14346 describes further methods for stabilizing mRNA. In particular, modifications of the mRNA are proposed which stabilize the mRNA species against degradation by RNAases. Such modifications relate, on the one hand, to stabilization by sequence modifications, in particular reducing the C and/or U content by base elimination or base substitution. On the other hand, chemical modifications are proposed, in particular the use of nucleotide analogues, together with 5′ and 3′ blocking groups, increased length of the poly-A tail and complexation of the mRNA with stabilizing means and combinations of the stated measures.

US patents U.S. Pat. No. 5,580,859 and U.S. Pat. No. 6,214,804 disclose mRNA vaccines and therapeutic agents inter alia in the context of “transient gene therapy” (TGT). Various measures are described for increasing translational efficiency and mRNA stability, which primarily relate to untranslated sequence domains.

Bieler and Wagner (in: Schleef (ed.), Plasmids for Therapy and Vaccination, Chapter 9, pages 147 to 168, Wiley-VCH, Weinheim, 2001) report on the use of synthetic genes in connection with gene therapy methods using of DNA vaccines and lentiviral vectors. The construction of a synthetic gag gene derived from HIV-1 is described, in which the codons have been modified relative to the wild-type sequence (alternative codon usage) in such a manner that it corresponded to the use of codons as is found in highly expressed mammalian genes. In this manner, the A/T content was in particular reduced relative to the wild-type sequence. The authors in particular found an increased expression rate of the synthetic gag gene in transfected cells. Increased formation of antibodies against the gag protein was furthermore observed in mice immunized with the synthetic DNA construct as was greater in vitro cytokine release in transfected mouse spleen cells. Finally, it was possible to see an induction of a cytotoxic immune response in mice immunized with gag expression plasmid. The authors of this article attribute the improved properties of their DNA vaccine substantially to a modification, brought about by the optimized codon use, of nucleo-cytoplasmic transport of the mRNA expressed by the DNA vaccine. In contrast, the authors consider the effect of modified codon usage on translational efficiency to be slight.

In the meantime, methods based on mRNA vaccination and compositions usable for this purpose, in which mRNA is preferably stabilized, have also been described in the prior art.

WO 02/098443 accordingly describes a pharmaceutical composition which contains a stabilized mRNA and is used as a vaccine for the treatment of cancers and infectious diseases and for tissue regeneration. The mRNA codes for a biologically active or antigenic peptide and is in particular stabilized by increasing the C/G content in the coding region.

WO 03/051401 describes a pharmaceutical composition which contains an mRNA coding for a tumor antigen and optionally contains a cytokine for the treatment and prevention of neoplastic diseases. In this case too, several variants are described for stabilizing the mRNA in this composition.

However, no mRNA vaccines have been described in the prior art which also ensure or increase or facilitate triggering of an immune response in the organism to which they are administered. This would, however, be highly advantageous, as the organism (patient) could or would have to be exposed to increased stress, for example due to repeated administration, increased dosages etc., if the mRNA vaccination did not proceed successfully or not to the desired extent. This also increases the risk of side effects against the vaccine occurring.

The object of the present invention is accordingly to provide a novel system for gene therapy or genetic vaccination which, on the one hand, overcomes the disadvantages of using DNA therapeutic agents and DNA vaccination and, on the other hand, achieves a more effective action of therapeutic agents and vaccines based on mRNA.

This object is achieved by the embodiments of the present invention defined in the claims.

The present invention accordingly provides a mixture which contains mRNA for vaccination, wherein at least one mRNA contains a domain which codes for at least one antigen from a tumor and at least one further mRNA contains a domain which codes for at least one immunogenic protein.

The invention is based on the recognition that virtually any organism exhibits “memory immune responses” against certain foreign molecules, for example proteins, in particular viral proteins, antigens. This means that an organism has already been infected at an earlier point in time with such a foreign molecule and that an immune response against this foreign molecule, for example a viral protein, has already been triggered by this infection and the immune system has a “memory” of this response, i.e. it stores it. On reinfection with the same foreign molecule, this immune response is reactivated. According to the invention, such reactivation of the immune response may proceed by vaccination with the mixture according to the invention, specifically by the mRNA present in the mixture, which mRNA contains a domain which codes for at least one immunogenic protein. According to the invention, this reactivation may even proceed in localized manner, namely at the point where the mixture is administered, for example administration into tumor tissue. In this manner, triggering of a (new) immune response against the above-described foreign molecule (against which there is a memory immune response) can be assisted/facilitated.

“Vaccination” or “inoculation” in general means the introduction of one or more antigens of a tumor or, for the purposes of the invention, the introduction of the genetic information for one or more antigen(s) of a tumor in the form of the mRNA which codes for the antigen(s) of a tumor into an organism, in particular into one or more cell(s) or tissue of this organism. In the organism or the cells thereof, the mRNA administered in this manner is translated into the (tumor) antigen, i.e. the antigen coded by the mRNA (also: antigenic polypeptide or antigenic peptide) is expressed, so stimulating an immune response directed against this antigen.

According to the present invention, an “antigen from a tumor” or also “tumor antigen” means that the corresponding antigen is expressed in cells which are associated with a tumor. In particular, these are antigens which are produced in the degenerate cells (tumor cells) themselves. These preferably comprise antigens which are located on the surface of the cells. Furthermore, those antigens from tumors which are expressed in cells which are not themselves degenerate or were not themselves originally degenerate but which are associated with the above above-mentioned tumor are also included according to the invention. These also include, for example, antigens related with tumor-supplying vessels or to the formation or neogenesis thereof, in particular such antigens which are associated with neovascularization or angiogenesis, for example growth factors such as VEGF, bFGF, etc. Such antigens associated with a tumor are furthermore also those antigens which originate from cells of the tissue in which the tumor is embedded. These may comprise, for example, antigens of connective tissue cells, for example antigens of the extracellular matrix. The mixture according to the invention may contain (at least one) mRNA which code(s) for 1 to 50, preferably 1 to 10 such antigens from a tumor.

Examples of such tumor antigens are 707-AP, AFP, ART-4 (adenocarcinoma recognized antigen; AB026125), BAGE, β-catenin/m, Bcr-abl, CAMEL (AJ012835), CAP-1, CASP-8, CDC27/m, CDK4/m, CEA, CT, Cyp-B, DAM, ELF2M, ETV6-AML1, G250, GAGE, for example GAGE-4, GnT-V, GP 100HAGE, HAGE, HAST-2, HLA-A*0201-R170I, HPV-E7, HSP70-2M, hTERT (or hTRT), iCE, KIAA0205, LAGE, for example LAGE-1, LDLR/FUT, MAGE, for example MAGE-A, MAGE-B, MAGE-C, MAGE-A1, MAGE-A2 (L18920), MAGE-A3, MAGE-A4 (U10687), MAGE-A6, MAGE-A10; MC1R (melanocyte stimulating hormone receptor; X65634), myosin/m, melan-A, melan-A/MART-1, Muc1, mucin-1, MUM-1, -2, -3, NA88-A, NY-ESO-1, NY-ESO-1/LAGE-2, p190 minor bcr-abl, Pml/RAR□, PRAME (U65011), proteinase 3, PSA, PSM, RAGE, for example RAGE-3 (U46193), RU1 or RU2, SAGE, SART-1 (AB006198), SART-2 (AF098066) or SART-3 (AB020880), SCP1, SSX, for example SSX2 (X86175), survivin, TEL/AML1, TPI/m, TRP-1, TRP-2, TRP-2/INT2, tyrosinase and WT1 (BC046461), VEGF (M32977), VEGFR-2 (AF063658), VEGFR-1 (XM_{—}497921), PDGF-R (BC032224), Her3 (M34309), Ep-CAM (KSA or GA733-2; M32325 or M33011), PSMA (AF007544), PSA (M26663), PSCA (AF043498), vimentin (Z19554), adipose differentiation antigen (X97324), β-actin (M10277), Met protooncogene 002958), isoform G250 of carbonic anhydrase (X66839), cytochrome P450 (AF450132), cyclin D1 (X59798), cyclin (M15796), DAM (X82539), HCV polyprotein (L20498), p53 (M14695), MDM2 (X58876), sperm protein (AF015527), adenovirus protein E3, α-actinin 4, CD4 cyclin-dependent protein kinase, KIAA 0020 (D13645), malic enzyme (L34035), MYO 1G, Pmel17 (M77348), Wegener's autoantigen (X56132), silencing information regulator 2-like protein (AF095714), ribosomal protein S2 (BC001795), multidrug resistance protein-3 (Y17151), adenovirus protein E1a, adenovirus E1b, Bcr-Abl, PR3, E/L-selectin, recoverin, hTERT, and CMV pp 65.

Particularly preferred tumor antigens are MAGE, in particular MAGE-A1 and MAGE-A6, melan-A, GP100, tyrosinase, survivin, CEA (carcino-embryonal antigen), Her-2/Neu and mucin-1. It is furthermore preferred if an RNA mixture according to the invention contains at least one viral tumor antigen (for example HPV-E7 or HCV polyprotein or adenovirus protein E3, E1a or E1b), optionally in combination with at least one preferably autologous tumor antigen of human origin from the patient to be treated. The autologous tumor antigen preferably comprises one of the above-stated antigens, in particular MAGE, specifically MAGE-A1 and MAGE-A6, melan-A, GP100, tyrosinase, survivin, CEA (carcino-embryonal antigen), Her-2/Neu, mucin-1, PSA, p53, Bcr-abl, PDGFR, Her3 or cyclin. An RNA mixture according to the invention very particularly preferably comprises one or two different viral tumor antigens in combination with 2 to 6 different autologous tumor antigens from the patient. In the case of an RNA mixture without viral tumor antigens, it is likewise preferred that said mixture contains 2 to 6 different tumor antigens, in particular selected from among the group of the above-stated tumor antigens.

In a preferred embodiment of the present invention the at least one mRNA of the mixture, which mRNA contains a domain which codes for at least one antigen from a tumor, codes for an antigen selected from the group consisting of MAGE, in particular MAGE-A1 and MAGE-A6, melan-A, GP100, tyrosinase and survivin.

A likewise preferred embodiment of the present invention relates to a mixture, wherein the at least one mRNA, which contains a domain which codes for at least one antigen from a tumor, codes for an antigen which is selected from the group consisting of MAGE, in particular MAGE-A1, CEA (carcino-embryonal antigen), Her-2/Neu, mucin-1 and survivin.

A further preferred embodiment of the present invention relates to a mixture, wherein the at least one mRNA, which contains a domain which codes for at least one antigen from a tumor, codes for an antigen from the group consisting of telomerase TERT, PR3, WT1, PRAME, mucin-1 and survivin.

In a further preferred embodiment of the present invention the at least one mRNA of the mixture, which mRNA contains a domain which codes for at least one antigen from a tumor, codes for an antigen selected from the group consisting of TNC (tenascin C), EGFR1, SOX9, SEC61G and PTPRZ1.

A further preferred embodiment of the present invention relates to a mixture, wherein the at least one mRNA, which contains a domain which codes for at least one antigen from a tumor, codes for an antigen selected from the group consisting of accession number M77481, accession number NM_{—}005363, accession number NM_{—}005511, accession number M77348, accession number NM_{—}000372 and accession number AF077350.

A likewise preferred embodiment of the present invention relates to a mixture, wherein the at least one mRNA, which contains a domain which codes for at least one antigen from a tumor, codes for an antigen selected from the group consisting of accession number M77481, accession number NM_{—}004363, accession number M11730, accession number NM_{—}002456 and accession number AF077350.

A further preferred embodiment of the present invention relates to a mixture, wherein the at least one mRNA, which contains a domain which codes for at least one antigen from a tumor, codes for an antigen selected from the group consisting of accession number NM_{—}003219, accession number NM_{—}002777, accession number NM_{—}000378, accession number NM_{—}006115, accession number NM_{—}002456 and accession number AF077350.

A furthermore preferred embodiment of the present invention relates to a mixture, wherein the at least one mRNA, which contains a domain which codes for at least one antigen from a tumor, codes for an antigen selected from the group consisting of accession number X78565, accession number AF288738, accession number Z46629, accession number NM_{—}014302 and accession number NM_{—}002851.

All accession numbers listed in the present invention relate to the respective protein sequences obtained from the NCBI (PubMed) database on the Internet at http://www.ncbi.nlm.nih.gov/entrez/query.fcgi (or http://www.ncbi.nlm.nih.gov/entrez/query.fcgi?db=Protein&itool=toolbar).

According to a further preferred embodiment the antigen(s) of a tumor is/are a polyepitope of the antigen(s) from a tumor. A “polyepitope” of an antigen or of two or more antigens is an amino acid sequence in which several or many regions of the antigen(s) which enter into interaction with the antigen-binding part of an antibody or with a T cell receptor are represented. The polyepitope may here be present in complete and unmodified form. According to the present invention, it may, however also be present in modified form, in particular to optimize antibody/antigen or T cell receptor/antigen interaction. A modification relative to the wild-type polyepitope may, for example, comprise a deletion, addition and/or substitution of one or more amino acid residues. Accordingly, in comparison with the mRNA coding for the wild-type polyepitope, one or more nucleotides is/are removed, added and/or replaced in the mRNA of the present invention coding for the modified polyepitope.

“Immunogenic protein” for the purposes of the invention relates to a “foreign protein”, in particular a “protein of a pathogen”, which triggers an immune response if it enters a foreign organism. The terms “immunogenic protein”, “foreign protein” and “protein of a pathogen” should be used as synonyms. Furthermore, the term “protein” is also synonymous for “polypeptide” and “peptide”. Such an immunogenic protein in particular comprises a viral or bacterial protein or a fungal protein. According to the invention, however, proteins from any other desired pathogen are included. The immune response is generally triggered by the infection of the foreign organism (for example a mammal, in particular a human) with a pathogenic organism, for example a virus, which contains this immunogenic protein or carries it on its surface and introduces it into the foreign organism by the infective process. It is preferred, once an organism has been infected with such an immunogenic protein, for the immune response triggered thereby to be stored in the organism and for this immune response to be reactivated in the event of renewed infection with this protein. A “memory” immune response against the immunogenic protein is thus present. One example of such a process is provided by a widespread virus with which for example virtually every adult, in particular human, individual has already been infected in his/her lifetime, specifically the influenza A or B virus. In the case of this infection, an immune response is formed against the influenza virus proteins, including the influenza matrix proteins. If such an influenza virus protein, in particular an influenza matrix protein, again gets into the already previously infected organism, the organism reactivates the immune response against the protein(s).

Immunogenic proteins for the purposes of the invention are preferably structural proteins of viruses, in particular matrix proteins, capsid proteins and surface proteins of the lipid membrane. Further examples of such viral proteins are proteins of adenoviruses, rhinoviruses, coronaviruses. The hepatitis B surface antigen (hereinafter denoted “HBs antigen”) is particularly preferred in this connection. The HBs antigen [accession number E00121] is a foreign antigen which constitutes a new antigen for most organisms, in particular mammals, especially humans, which have neither been infected with the hepatitis B virus (HBV) nor been vaccinated against HBV. An immune response to foreign antigens is generally detected more effectively than is an immune response to own antigens, such as tumor antigens, since cells which bear these endogenous antigens are usually inactivated or destroyed by the immune system in order to avoid autoimmunity. An immune response to the HBs antigen may accordingly serve as a surrogate marker for the efficiency of the administered mixture according to the invention. Furthermore, in cooperation with a further immunogenic protein of the invention the HBs antigen can considerably amplify the immune response of the organism to which the mixture according to the invention is administered. Another preferred immunogenic protein is CMV pp 65 [accession number M15120].

One very particularly preferred immunogenic protein is the influenza matrix protein, more specifically influenza matrix protein M1. Two types of the influenza virus are known, influenza A virus and influenza B virus. Various serotypes, each exhibiting slight sequence differences between one another, are known for both types. A preferred embodiment of the invention accordingly relates to a mixture in which the at least one mRNA, which contains a domain which codes for at least one immunogenic protein or polypeptide, codes for a matrix protein, preferably an influenza matrix protein, particularly preferably influenza A matrix protein M1 or influenza B matrix protein M1.

Consequently, a preferred embodiment of the present invention relates to a mixture in which the at least one mRNA, which contains a domain which codes for at least one immunogenic protein, codes for a matrix protein, preferably an influenza matrix protein, particularly preferably influenza A matrix protein M1 or influenza B matrix protein M1, or for HBs or CMV pp 65.

A further preferred embodiment of the present invention relates to a mixture, wherein the at least one mRNA, which contains a domain which codes for at least one immunogenic protein, codes for an immunogenic protein selected from the group consisting of accession number AF348197, accession number V01099, accession number E00121 and accession number M15120.

Examples of preferred immunogenic proteins according to the invention are proteins of widespread pathogens, i.e. pathogens with which every organism, in particular mammal, preferably human, has a high probability of being infected at least once in his/her lifetime. These include, for example, any structural or nonstructural protein of:
influenza virus type A or B or any other orthomyxovirus (influenza type C),
picornaviruses, such as rhinovirus or hepatitis A virus,
togaviruses, such as alphavirus or rubivirus, for example Sindbis, Semliki Forest or rubeola virus (measles virus), rubella virus (German measles virus),
coronaviruses, in particular subtypes HCV-229E or HCV-OC43,
rhabdoviruses, such as rabies virus,
paramyxoviruses, such as mumps virus,
reoviruses, such as group A, B or C rotavirus,
hepadnaviruses, such as hepatitis B virus,
papoviruses, such as human papillomaviruses (HPV) of any serotype (from 1 to 75),
adenoviruses from type 1 to 47,
herpesviruses, such as herpes simplex virus 1, 2 or 3, cytomegalovirus (CMV), particularly preferably CMVpp65, or Epstein-Barr virus (EBV),
vacciniaviruses and
the bacterium Chlamydophila pneumoniae (Chlamydia pneumoniae).

Examples of immunogenetic proteins likewise preferred according to the invention are proteins of pathogens which seldom infect an organism, in particular a mammal, preferably a human. These include, for example, any structural or nonstructural protein of:
flaviviruses, such as dengue virus types 1 to 4, yellow fever virus, West Nile virus, Japanese encephalitis virus or hepatitis C virus
caliciviruses,
filoviruses, such as Ebola virus,
bornaviruses,
bunyaviruses, such as Rift Valley fever virus,
arenaviruses, such as LCMV (lymphocytic choriomeningitis virus) or hemorrhagic fever viruses,
retrovirus, such as HIV and
parvoviruses.

According to the invention, functional fragments and/or functional variants of an immunogenic protein or of an antigen from a tumor of the invention and the mRNA according to the invention are likewise included. For the purposes of the invention “functional” means that the immunogenic protein or the antigen from a tumor or the mRNA exhibits immunological or immunogenic activity, in particular triggers an immune response in an organism in which it is foreign. The mRNA according to the invention is functional if it can be translated into a functional immunogenic protein or tumor antigen (or fragment thereof).

For the purposes of the invention, a “fragment” should be taken to mean a truncated immunogenic protein or tumor antigen or a truncated mRNA of the present invention. These may comprise N-terminally, C-terminally or intrasequentially truncated amino acid or nucleic acid sequences.

The production of fragments according to the invention is well known in the prior art and may be carried out by a person skilled in the art using standard methods (see for example Maniatis et al. (2001), Molecular Cloning: Laboratory Manual, Cold Spring Harbor Laboratory Press). The fragments of the immunogenic protein or of the antigen may in general be produced by modifying the DNA sequence which codes for the wild-type molecule, followed by transformation of this DNA sequence into a suitable host and expression of this modified DNA sequence, on condition that modification of the DNA does not destroy the described functional activities. In the case of the mRNA according to the invention, production of the fragment may likewise proceed by modifying the wild-type DNA sequence followed by in vitro transcription and isolation of the mRNA, likewise on condition that modification of the DNA does not destroy the functional activity of the mRNA. A fragment according to the invention may be identified, for example, by sequencing the fragments and subsequently comparing the sequence obtained with the wild-type sequence. Sequencing may proceed by standard methods, many of which are well known in the prior art.

For the purposes of the invention, “variants” are in particular those immunogenic proteins, antigens or mRNA which exhibit sequence differences relative to the corresponding wild-type sequences. These sequence deviations may comprise one or more insertion(s), deletion(s) and/or substitution(s) of amino acids or nucleic acids, a sequence homology of at least 60%, preferably 70%, more preferably 80%, likewise more preferably 85%, still more preferably 90% and most preferably 97% prevailing.

The percentage identity of two nucleic acid or amino acid sequences may be determined by aligning the sequences so that they may then be compared with one another. To this end, gaps may for example be introduced into the sequence of the first amino acid or nucleic acid sequence and the amino acids or nucleic acids at the corresponding position in the second amino acid or nucleic acid sequence may be compared. If a position in the first amino acid sequence is occupied with the same amino acid or the same nucleic acid as is the case in the second sequence, then the two sequences are identical at this position. The percentage identity between two sequences is a function of the number of identical positions divided by the sequences.

The percentage identity of two sequences may be determined with the assistance of a mathematical algorithm. One preferred, but non-limiting example of a mathematical algorithm which may be used for comparing two sequences, is the algorithm of Karlin et al. (1993), PNAS USA, 90:5873-5877. Such an algorithm is incorporated in the NBLAST software with which it is possible to identify sequences which exhibit a desired identity with the sequences of the present invention. A gapped alignment, as described above, may be obtained by using the “Gapped BLAST” software as described in Altschul et al. (1997), Nucleic Acids Res, 25:3389-3402.

For the purposes of the invention, functional variants may preferably be mRNA molecules which exhibit a stability and/or translation rate which is increased relative to the wild-type molecules. Better transport into the (host) organism cell may also be present. Variants may in particular also be immunogenic proteins which are stabilized in order to escape physiological degradation, for example by stabilization of the protein backbone by substitution of the amide-like bond, for example also by using β-amino acids.

The term variants in particular includes those amino acid sequences exhibiting conservative substitution relative to the physiological sequences. Conservative substitutions are defined as those substitutions in which amino acids originating from the same class are exchanged for one another. In particular, there are amino acids with aliphatic side chains, positively or negatively charged side chains, aromatic groups in the side chains or amino acids, the side chains of which can enter into hydrogen bridges, for example side chains which have a hydroxy function. This means that, for example an amino acid with a polar side chain is replaced by another amino acid with a likewise polar side chain or for example an amino acid characterized by a hydrophobic side chain is substituted by another amino acid with a likewise hydrophobic side chain (for example serine (threonine) by threonine (serine) or leucine (isoleucine) by isoleucine (leucine)). Insertions and substitutions are in particular possible at those sequence positions which do not bring about a change in three-dimensional structure or affect the binding domain. Modification of a three-dimensional structure by insertion(s) or deletion(s) may straightforwardly be verified, for example, with the assistance of CD spectra (circular dichroism spectra) (Urry, 1985, Absorption, circular dichroism and ORD of polypeptides, in: Modern Physical Methods in Biochemistry, Neuberger et al. (eds.), Elsevier, Amsterdam).

Variants in which “alternative codon usage” occurs are likewise included. Each amino acid is coded by a codon which is in each case defined by three nucleotides (a triplet). It is possible to exchange a codon which codes for a specific amino acid for another codon which codes for the same amino acid. The stability of the mRNA according to the invention may, for example, be increased by selecting suitable alternative codons. This is addressed in greater detail below.

Suitable methods for the production of variants according to the invention having amino acid sequences which comprise substitutions relative to the wild-type sequence are disclosed, for example, in documents U.S. Pat. No. 4,737,462, U.S. Pat. No. 4,588,585, U.S. Pat. No. 4,959,314, U.S. Pat. No. 5,116,943, U.S. Pat. No. 4,879,111 and U.S. Pat. No. 5,017,691. The production of variants in general is in particular also described by Maniatis et al., (2001), Molecular Cloning: A Laboratory Manual, Cold Spring Harbor Laboratory Press). Codons may here be omitted, added or exchanged. Variants for the purposes of the invention may likewise be produced by introducing modifications into the nucleic acids which code for the variants, such as for example insertions, deletions and/or substitutions of one or more nucleotides. Numerous methods for such modifications of nucleic acid sequences are known in the prior art. One of the most used techniques is oligonucleotide-directed site-specific mutagenesis (see Comack B., Current Protocols in Molecular Biology, 8.01-8.5.9, Ausubel F. et al., 1991 edition). In this technique, an oligonucleotide whose sequence comprises a specific mutation is synthesized. This oligonucleotide is then hybridized with a template which contains the wild-type nucleic acid sequence. A single-stranded template is preferably used in this technique. Once the oligonucleotide and template have been annealed, a DNA-dependent DNA polymerase is used in order to synthesize the second strand of the oligonucleotide which is complementary to the template DNA strand. As a result, a heteroduplex molecule is obtained which contains a mismatch pair arising from the above-mentioned mutation in the oligonucleotide. The oligonucleotide sequence is introduced into a suitable plasmid, which is in turn introduced into a host cell and the oligonucleotide DNA is replicated in this host cell. Using this technique, nucleic acid sequences with deliberate modifications (mutations) are obtained which can be used for the production of variants according to the invention.

The present invention may advantageously be used in the treatment and/or prevention of tumor disease and particularly preferably in the treatment and/or prevention of melanomas, carcinomas, AML (acute myeloid leukemia) and glioma. To this end, vaccination may be performed with the mixture according to the invention, wherein the mRNA which codes for an antigen codes for two or more different antigens, which are specific for melanomas (for example MAGE-A1, MAGE-A6, melan-A, GP100, tyrosinase and survivin) or specific for carcinomas (for example MAGE-A1, CEA, Her-2/Neu, mucin-1 and survivin) or specific for AML (for example telomerase TERT, PR3, WT1, PRAME, mucin-1 and survivin) or specific for glioma (for example TNC (tenascin C), EGFR1 (epidermal growth factor receptor 1), SOX9, SEC61G and PTPRZ1 (protein tyrosine phosphatase, receptor-type, Z polypeptide 1). In this manner, it is ensured according to the invention that a melanoma or carcinoma or AML or glioma can be combated more effectively as the combination of various antigens specific for the specific tumor exhibit an extremely broad spectrum of action. As already described, the particular mixture furthermore contains an mRNA which codes for an immunogenic protein, which mRNA preferably mediates the reactivation of an immune response. An influenza matrix protein, specifically an influenza A or B matrix protein M1, is in particular preferred according to the invention. The particular mixture may additionally contain the immunogenic protein HBs.

A particularly preferred embodiment of the invention accordingly relates to a mixture in which the at least one mRNA, which contains a domain which codes for at least one antigen from a tumor, codes for the antigens MAGE-A1 [accession number M77481], MAGE-A6 [accession number NM_{—}005363], melan-A [accession number NM_{—}005511], GP100 [accession number M77348], tyrosinase [accession number NM_{—}000372] and survivin [accession number AF077350] and the at least one mRNA, which contains a domain which codes for at least one immunogenic protein or polypeptide, codes for an influenza matrix protein [accession number AF348197 or accession number V01099]. The mixture preferably contains functional fragments and/or functional variants of the above-stated mRNAs.

A likewise particularly preferred embodiment of the invention consequently relates to a mixture, in which the at least one mRNA, which contains a domain which codes for at least one antigen from a tumor, codes for the antigens MAGE-A1 [accession number M77481], CEA [accession number NM_{—}004363], Her-2/Neu [accession number M11730], mucin-1 [accession number NM_{—}002456] and survivin [accession number AF077350], and the at least one mRNA, which contains a domain which codes for at least one immunogenic protein or polypeptide, codes for an influenza matrix protein [accession number AF348197 or accession number V01099]. The mixture preferably contains functional fragments and/or functional variants of the above-stated mRNAs.

A further particularly preferred embodiment of the invention relates to a mixture, in which the at least one mRNA, which contains a domain which codes for at least one antigen from a tumor, codes for the antigens telomerase TERT [accession number NM_{—}003219], PR3 [accession number NM_{—}002777], WT1 [accession number NM_{—}000378], PRAME [accession number NM_{—}006115], mucin-1[accession number NM_{—}002456] and survivin [accession number AF077350] and the at least one mRNA, which contains a domain which codes for at least one immunogenic protein or polypeptide, codes for an influenza matrix protein [accession number AF348197 or accession number V01099]. The mixture preferably contains functional fragments and/or functional variants of the above-stated mRNAs.

A further particularly preferred embodiment of the invention relates to a mixture, in which the at least one mRNA, which contains a domain which codes for at least one antigen from a tumor, codes for the antigens TNC (tenascin C) [accession number X78565], EGFR1 (“epidermal growth factor receptor 1”) [accession number AF288738], SOX9 [accession number Z46629], SEC61G [accession number NM_{—}014302] and PTPRZ1 (protein tyrosine phosphatase, receptor-type, Z polypeptide 1) [accession number NM_{—}002851] and the at least one mRNA, which contains a domain which codes for at least one immunogenic protein or polypeptide, codes for an influenza matrix protein [accession number AF348197 or accession number V01099]. The mixture preferably contains functional fragments and/or functional variants of the above-stated mRNAs.

A preferred embodiment relates to a mixture, in which the at least one mRNA, which contains a domain which codes for at least one immunogenic protein or polypeptide, codes for a matrix protein, preferably an influenza matrix protein [accession number AF348197 or accession number V01099], particularly preferably the influenza A matrix protein M1 or the influenza B matrix protein M1, and for an HBs antigen [accession number E00121]. The hepatitis B surface antigen is, as described above, particularly suitable for use in anti-viral vaccination.

The mRNA of the mixture according to the invention may be present as naked mRNA and/or as modified mRNA, in particular stabilized mRNA. Modification of the mRNA according to the invention above all serves to increase the stability of the mRNA but also to enhance the transfer of mRNA into a cell or a tissue of an organism. The mRNA of the mixture according to the invention preferably comprises one or more modifications, in particular chemical modifications, which contribute to increasing the half-life of the mRNA in the organism or enhance the transfer of mRNA into the cell or a tissue.

In a particularly preferred embodiment of the present invention, the G/C content of the coding domain of the modified mRNA of the mixture according to the invention is increased relative to the G/C content of the coding domain of the wild-type RNA, the coded amino acid sequence of the modified mRNA preferably not being modified relative to the coded amino acid sequence of the wild-type mRNA.

This modification is based on the fact that the sequence order of the mRNA domain to be translated is essential for efficient mRNA translation. The composition and the order of the various nucleotides are of significance here. In particular, sequences with an elevated G (guanosine)/C (cytosine) content are more stable than sequences with an elevated A (adenosine)/U (uracil) content. Thus, according to the invention, while retaining the translated amino acid sequence, the codons are varied relative to the wild-type mRNA in such a manner that they have a greater content of G/C nucleotides. Due to the fact that several codons code for one and the same amino acid (“degeneration of the genetic code”), it is possible to determine the codons which are most favorable for stability (“alternative codon usage”).

Depending on the amino acid to be coded by the modified mRNA, there are various possible options for modifying the mRNA sequence relative to the wild-type sequence. No modification of the codons is necessary in the case of amino acids which are coded by codons exclusively containing G or C nucleotides. Accordingly, the codons for Pro (CCC or CCG), Arg (CGC or CGG), Ala (GCC or GCG) and Gly (GGC or GGG) require no change because no A or U is present.

In contrast, codons which contain A and/or U nucleotides may be modified by substitution of other codons which code for the same amino acids, but contain no A and/or U. Examples of these are:
the codons for Pro may be changed from CCU or CCA to CCC or CCG;
the codons for Arg may be changed from CGU or CGA or AGA or AGG to CGC or CGG;
the codons for Ala may be changed from GCU or GCA to GCC or GCG;
the codons for Gly may be changed from GGU or GGA to GGC or GGG.

In other cases, while A or U nucleotides cannot be eliminated from the codons, it is however possible to reduce the A and U content by using codons containing a smaller proportion of A and/or U nucleotides. Examples of these are:
the codons for Phe may be changed from UUU to UUC;
the codons for Leu may be changed from UUA, UUG, CUU or CUA to CUC or CUG;
the codons for Ser may be changed from UCU or UCA or AGU to UCC, UCG or AGC;
the codon for Tyr may be changed from UAU to UAC;
the codon for Cys may be changed from UGU to UGC;
the codon for H is may be changed from CAU to CAC;
the codon for Gln may be changed from CAA to CAG;
the codons for Ile may be changed from AUU or AUA to AUC;
the codons for Thr may be changed from ACU or ACA to ACC or ACG;
the codon for Asn may be changed from AAU to AAC;
the codon for Lys may be changed from AAA to AAG;
the codons for Val may be changed from GUU or GUA to GUC or GUG;
the codon for Asp may be changed from GAU to GAC;
the codon for Glu may be changed from GAA to GAG,
the stop codon UAA may be changed to UAG or UGA.

In the case of codons for Met (AUG) and Trp (UGG), in contrast, there is no possibility of sequence modification.

The above listed substitutions may be used both individually and in any possible combinations to increase the G/C content of the modified mRNA relative to the wild-type mRNA (the original sequence). Thus, for example, all codons for Thr occurring in the wild-type sequence may be changed to ACC (or ACG). Preferably, however, combinations of the above substitution options are, for example, used:
substitution of all the codons coding for Thr in the original sequence (wild-type mRNA) with ACC (or ACG) and substitution of all the codons originally coding for Ser with UCC (or UCG or AGC);
substitution of all the codons coding for Ile in the original sequence with AUC and substitution of all the codons originally coding for Lys with AAG and substitution of all the codons originally coding for Tyr with UAC;
substitution of all the codons coding for Val in the original sequence with GUC (or GUG) and substitution of all the codons originally coding for Glu with GAG and substitution of all the codons originally coding for Ala with GCC (or GCG) and substitution of all the codons originally coding for Arg with CGC (or CGG);
substitution of all the codons coding for Val in the original sequence with GUC (or GUG) and substitution of all the codons originally coding for Glu with GAG and substitution of all the codons originally coding for Ala with GCC (or GCG) and substitution of all the codons originally coding for Gly with GGC (or GGG) and substitution of all the codons originally coding for Asn with AAC;
substitution of all the codons coding for Val in the original sequence with GUC (or GUG) and substitution of all the codons originally coding for Phe with UUC and substitution of all the codons originally coding for Cys with UGC and substitution of all the codons originally coding for Leu with CUG (or CUC) and substitution of all the codons originally coding for Gln with CAG and substitution of all the codons originally coding for Pro with CCC (or CCG);
   etc.

The G/C content of the modified mRNA domain which codes for the protein is preferably increased by at least 7 percentage points, more preferably by at least 15 percentage points, particularly preferably by at least 20 percentage points relative to the G/C content of the coded domain of the wild-type mRNA which codes for the protein.

It is particularly preferred in this connection to maximally increase the G/C content of the modified mRNA, in particular in the domain which codes for the protein, in comparison with the wild-type sequence. G/C-maximized sequences for the coding domains of a preferred selection of viral or tumor antigens which may be used in an RNA mixture according to the invention are shown in FIGS. 19 to 81.

A further preferred modification of the mRNA of the mixture according to the invention is based on the recognition that translational efficiency is likewise determined by a differing frequency in the occurrence of tRNAs in cells. Accordingly, if a larger number of “rare” codons are present in an RNA sequence, the corresponding mRNA is distinctly worse translated than when codons coding for relatively “frequent” tRNAs are present.

Thus, according to the invention, in the modified mRNA of the mixture according to the invention, the domain which codes for the protein, peptide or polypeptide is modified relative to the corresponding domain of the wild-type mRNA in such a manner that at least one codon of the wild-type sequence, which codes for a relatively rare tRNA in the cell, is exchanged for a codon which codes for a tRNA which is relatively frequent and carries the same amino acid as the relatively rare tRNA. This modification changes the RNA sequences in such a manner that codons are inserted for which frequently occurring tRNAs are available. In other words, according to the invention, it is possible by this modification in each case to exchange all the codons of the wild-type sequence which code for a relatively rare tRNA in the cell for a codon which codes for a relatively frequent tRNA in the cell, the relatively frequent tRNA in each case carrying the same amino acid as the relatively rare tRNA.

The person skilled in the art is aware of which tRNAs occur relatively frequently in the cell and which, in comparison, occur relatively rarely; c.f. for example Akashi, Curr. Opin. Genet. Dev. 2001, 11(6): 660-666.

It is particularly preferred according to the invention to associate the G/C sequence content which, according to the invention, has been increased, in particular maximized, in the modified mRNA with the “frequent” codons, without modifying the amino acid sequence of the protein, peptide or polypeptide coded by the coding domain of the mRNA. This preferred embodiment provides a particularly efficiently translated and stabilized mRNA for example for the mixture according to the invention.

Identification of an mRNA modified in the above-described manner (increase in G/C content; exchange of tRNAs) may be achieved by using the computer program explained in WO 02/098443, the full disclosure of which is incorporated into the present invention. With this computer software it is possible, on the basis of the genetic code or the degeneracy thereof, to modify the nucleotide sequence of any desired mRNA in such a manner that a maximum G/C content is obtained in conjunction with the use of codons which code for tRNAs which occur maximally frequently in the cell, the amino acid sequence coded by the modified mRNA preferably not being modified relative to the unmodified sequence. Alternatively, it is also possible to modify only the G/C content or only the codon usage relative to the original sequence. The source code in Visual Basic 6.0 (development environment used: Microsoft Visual Studio Enterprise 6.0 with service pack 3) is likewise stated in WO 02/098443.

In a further preferred embodiment of the present invention, the A/U content in the surroundings of the ribosome binding site of the modified mRNA of the mixture according to the invention is increased relative to the A/U content in the surroundings of the ribosome binding site of the wild-type mRNA. This modification (increased A/U content around the ribosome binding site) increases the effectiveness of ribosome binding to the mRNA. Effective binding of the ribosomes to the ribosome binding site (Kozak sequence: GCCGCCACCAUGG, the AUG forms the start codon) in turn ensures efficient translation of the mRNA.

A likewise preferred embodiment of the present invention relates to a mixture according to the invention, wherein the coding domain and/or the 5′- and/or 3′-untranslated domain of the modified mRNA is modified relative to the wild-type mRNA in such a manner that it contains no destabilizing sequence elements, the coded amino acid sequence of the modified mRNA preferably not being modified relative to the wild-type mRNA. It is known that destabilizing sequence elements (DSEs) occur for example in the sequences of eukaryotic mRNAs and signal proteins bind to these elements and regulate the enzymatic degradation of mRNA in vivo. Accordingly, in order to provide further stabilization of the modified mRNA according to the invention, it is optionally possible to make one or more such modifications in the domain which codes for the protein relative to the corresponding domain of the wild-type mRNA, such that no or substantially no destabilizing sequence elements are present there. According to the invention, DSEs present in the untranslated domains (3′- and/or 5′-UTR) may likewise be eliminated from the mRNA by such modifications.

Such destabilizing sequences are for example AU-rich sequences (“AURES”), which occur in 3′-UTR fragments of numerous unstable mRNAs (Caput et al., Proc. Natl. Acad. Sci. USA 1986, 83: 1670 to 1674). The mRNA molecules contained in the mixture according to the invention are thus preferably modified relative to the wild-type mRNA in such a manner that they comprise no such destabilizing sequences. This also applies to such sequence motifs which are recognized by possible endonucleases, for example the sequence GAACAAG, which is present in the 3′ UTR segment of the gene which codes for the transferrin receptor (Binder et al., EMBO J. 1994, 13: 1969 to 1980). These sequence motifs are also preferably removed from the modified mRNA of the mixture according to the invention.

In a further preferred embodiment of the present invention, the modified mRNA of the mixture according to the invention comprises a 5′ cap structure. Examples of cap structures which may be used according to the invention are m7G(5′)ppp (5′(A,G(5′)ppp(5′)A and G(5′)ppp(5′)G.

It is furthermore preferred for the modified mRNA of the mixture according to the invention to comprise a poly(A) tail, preferably of at least 25 nucleotides, more preferably of at least 50 nucleotides, still more preferably of at least 70 nucleotides, likewise more preferably of at least 100 nucleotides, most preferably of at least 200 nucleotides.

The modified mRNA of the mixture according to the invention likewise preferably comprises at least one IRES and/or at least one 5′ and/or 3′ stabilization sequence. According to the invention, one or more IRES (internal ribosomal entry sites) may thus be inserted into the modified mRNA. An IRES may accordingly act as a sole ribosome binding site, but may also serve to provide an mRNA which codes for two or more proteins, peptides or polypeptides which are to be mutually independently translated by the ribosomes (“multicistronic mRNA”). Examples of IRES sequences which are usable according to the invention are those from picornaviruses (for example FMDV), pestiviruses (CFFV), polioviruses (PV), encephalomyocarditis viruses (ECMV), foot and mouth disease viruses (FMDV), hepatitis C viruses (HCV), classical swine fever viruses (CSFV), murine leukemia virus (MLV), simian immunodeficiency viruses (SIV) or cricket paralysis viruses (CrPV).

The modified mRNA of the mixture according to the invention furthermore preferably comprises at least one 5′ and/or 3′ stabilization sequence. These stabilization sequences in the 5′ and/or 3′ untranslated domains bring about an increase in the half-life of the mRNA in the cytosol. These stabilization sequences may exhibit 100% sequence homology to naturally occurring sequences which occur in viruses, bacteria and eukaryotes, but may also be of a partially or completely synthetic nature. Examples of stabilizing sequences usable in the present invention which may be mentioned are the untranslated sequences (UTR) of □-globin gene, for example of Homo sapiens or Xenopus laevis. Another example of a stabilization sequence exhibits the general formula (C/U)CCANₓCCC(U/A)PyₓUC(C/U)CC, which is present in the 3′UTR of the very stable mRNA, which codes for □-globin, □-(I)-collagen, 15-lipoxygenase or for tyrosine hydroxylase (c.f. Holcik et al., Proc. Natl. Acad. Sci. USA 1997, 94: 2410 to 2414). It goes without saying that such stabilization sequences may be used not only individually or in combination but also in combination with other stabilization sequences known to a person skilled in the art.

In a preferred embodiment of the present invention, the modified mRNA of the mixture according to the invention comprises at least one analogue of naturally occurring nucleotides. This/these analogue/analogues serve(s) to provide further stabilization of the modified mRNA, this stabilization being based on the fact that the RNA-degrading enzymes occurring in the cells preferentially recognize naturally occurring nucleotides as a substrate. RNA degradation may thus be made more difficult by insertion of nucleotide analogues in the RNA, the effect on translational efficiency on insertion of these analogues, in particular into the coding domain of the mRNA, possibly having a positive or negative effect on the translational efficiency. Examples of nucleotide analogues which are usable according to the invention and may be mentioned in a non-exhaustive list are phosphoramidates, phosphorthioates, peptide nucleotides, methylphosphonates, 7-deazaguanosine, 5-methylcytosine and inosine. The production of such analogues is known to a person skilled in the art for example from US patents U.S. Pat. No. 4,373,071, U.S. Pat. No. 4,401,796, U.S. Pat. No. 4,415,732, U.S. Pat. No. 4,458,066, U.S. Pat. No. 4,500,707, U.S. Pat. No. 4,668,777, U.S. Pat. No. 4,973,679, U.S. Pat. No. 5,047,524, U.S. Pat. No. 5,132,418, U.S. Pat. No. 5,153,319, U.S. Pat. Nos. 5,262,530 and 5,700,642. According to the invention, such analogues may occur in untranslated and translated domains of the modified mRNA.

The modified mRNA of the mixture according to the invention, which contains a domain which codes for at least one antigen from a tumor, may preferably additionally contain a further functional fragment, which codes, for example, for a cytokine which promotes the immune response (monokine, lymphokine, interleukin or chemokine, such as IL-1, IL-2, IL-3, IL-4, IL-5, IL-6, IL-7, IL-8, IL-9, IL-10, IL-12, INF-α, INF-α, GM-CFS, LT-α) or growth factors, such as hGH.

Various methods for carrying out the described modifications are familiar to a person skilled in the art. Some of these methods have already been described in the above section relating to variants of the invention. For example, in the case of relatively short coding domains (which code for biologically active or antigenic proteins or peptides), codons may be substituted in the modified mRNA according to the invention by synthesizing the entire mRNA chemically using standard techniques.

Preferably, however, base substitutions, additions or eliminations are effected using a DNA template for producing the modified mRNA with the assistance of conventional techniques of targeted mutagenesis (see for example Maniatis et al., Molecular Cloning: A Laboratory Manual, Cold Spring Harbor Laboratory Press, 3rd ed., Cold Spring Harbor, N.Y., 2001). In such a method, the mRNA is produced by in vitro transcription of a corresponding DNA molecule. This DNA template has a suitable promoter, for example a T7 or SP6 promoter, for in vitro transcription, which is followed by the desired nucleotide sequence for the mRNA to be produced and a termination signal for the in in vitro transcription. According to the invention, the DNA molecule, which forms the template for the RNA construct to be produced, is produced by fermentative multiplication and subsequent isolation as part of a plasmid which is replicable in bacteria. Examples of suitable plasmids for the present invention which may be mentioned are the plasmids pT7Ts (GenBank accession number U26404; Lai et al., Development 1995, 121: 2349 to 2360), pGEM® series, for example pGEM®-1 (GenBank accession number X65300; from Promega) and pSP64 (GenBank accession number X65327); c.f. also Mezei and Storts, Purification of PCR Products, in: Griffin and Griffin (eds.), PCR Technology: Current Innovation, CRC Press, Boca Raton, Fla., 2001.

In this manner, using short synthetic DNA oligonucleotides, which comprise short single-stranded transitions at the resultant restriction sites, or genes produced by chemical synthesis, it is possible to clone the desired nucleotide sequence into a suitable plasmid in accordance with molecular biological methods familiar to a person skilled in the art (c.f. Maniatis et al., above). The DNA molecule is then excised from the plasmid, in which it may be present in a single copy or multiple copies, by digestion with restriction endonucleases.

In a further embodiment of the present invention, the modified mRNA of the mixture according to the invention is complexed or condensed with at least one cationic or polycationic agent. Preferably, such a cationic or polycationic agent is an agent which is selected from the group consisting of protamine, poly-L-lysine, poly-L-arginine and histones.

This modification of the mRNA according to the invention makes it possible to improve the effective transfer of the modified mRNA into the cells or tissue or organism to be treated by the modified mRNA being associated with or bound onto a cationic peptide or protein. In particular, protamine is particularly effectively used here as a polycationic, nucleic acid-binding protein. It is, of course, also possible to use other cationic peptides or proteins, such as poly-L-lysine or histones. This procedure for stabilizing the modified mRNA is described, for example, in EP-A-1083232, the disclosure of which in this respect is included in its entirety in the present invention.

All the above-described modifications of the mRNA of the mixture according to the invention may occur for the purposes of the invention individually or in combination with one another.

The present invention further provides a mixture according to the invention for use as a pharmaceutical composition.

The present invention further provides a pharmaceutical composition which contains a mixture according to the invention as well as pharmaceutically suitable auxiliary substances and/or excipients. A combination of the mRNAs according to the invention with pharmaceutically acceptable excipients, auxiliary substances and/or additives is accordingly also disclosed according to the invention. Corresponding production methods are disclosed in “Remington's Pharmaceutical Sciences” (Mack Pub. Co., Easton, Pa., 1980), which is part of the disclosure of the present invention. The pharmaceutical composition of the present invention preferably additionally contains at least one RNase inhibitor, preferably RNasin.

Excipients which may be considered for parenteral administration are, for example, sterile water, sterile saline solutions, polyalkylene glycols, hydrogenated naphthalene and in particular biocompatible lactide polymers, lactide/glycolide copolymer or polyoxyethylene/polyoxypropylene copolymers. Pharmaceutical compositions according to the invention may contain fillers or substances, such as lactose, mannitol, substances for covalent linkage of polymers, such as for example polyethylene glycol, onto inhibitors according to the invention, complexation with metal ions or inclusion of materials in or on particular polymer compound preparations, such as for example polylactate, polyglycolic acid, hydrogel or onto liposomes, microemulsion, micelles, unilamellar or multilamellar vesicles, erythrocyte fragments or spheroplasts. The particular embodiments of the pharmaceutical composition are selected depending on physical behavior, for example with regard to solubility, stability, bioavailability or degradability. Controlled or constant release of the active ingredient component according to the invention in the composition includes formulations on the basis of lipophilic depots (for example fatty acids, waxes or oils). Coatings of substances or compositions according to the invention containing such substances, namely coatings with polymers (for example poloxamers or poloxamines) are also disclosed for the purposes of the present invention. Substances or compositions according to the invention may furthermore comprise protective coatings, for example protease inhibitors or permeability enhancers. Preferred carriers are typically aqueous carrier materials, with water for injection (WFI) or water buffered with phosphate, citrate or acetate etc. being used, and the pH typically being adjusted to 5.0 to 8.0, preferably 6.0 to 7.0. The carrier or vehicle will additionally preferably contain salt constituents, for example sodium chloride, potassium chloride or other components, which for example make the solution isotonic. Apart from the above-stated constituents, the carrier may furthermore contain additional components, such as human serum albumin (HSA), polysorbate 80, sugar or amino acids.

The manner of administration and the dosage of the pharmaceutical composition according to the invention depend not only on the complaint to be treated and its stage of progression, but also on the patient's body weight, age and gender. The concentration of the modified mRNA in such formulations may accordingly vary within a wide range from 1 μg to 100 mg/ml. The pharmaceutical composition according to the invention is preferably administered to the patient parenterally, for example intravenously, intraarterially, subcutaneously, intramuscularly. The pharmaceutical composition may likewise be administered topically or orally.

The present invention consequently likewise provides a method for the treatment of diseases, in particular neoplastic or tumor diseases or a vaccination for prevention of the above-stated diseases, which method comprises the administration of the pharmaceutical composition according to the invention to a patient, in particular a human.

It is preferred according to the invention for the pharmaceutical composition of the present invention furthermore to contain one or more adjuvant(s) by which means an increase in the immunogenicity of the pharmaceutical composition may be effected. According to the invention, an “adjuvant” should be taken to mean any chemical or biological compound which promotes a specific immune response. Depending on the various kinds of adjuvants, different mechanisms may be considered in this respect. For example, compounds which promote endocytosis of the modified mRNA present in the pharmaceutical composition by dendritic cells (DC) form a first class of usable adjuvants. Other compounds which permit maturation of DCs, for example lipopolysaccharides, TNF-□ or CD40 ligand, are another class of suitable adjuvants. In general, any kind of agent having an action on the immune system in the manner of a “danger signal” (LPS, GP96, oligonucleotides with the CpG motif) or cytokines, such as GM-CFS, may be used as an adjuvant which make it possible to increase and/or to purposefully influence an immune response against an antigen which is coded by the modified mRNA. In particular, the above-stated cytokines are preferred here. Further known adjuvants are aluminum hydroxide, Freud's adjuvant as well as the above-stated stabilizing cationic peptides or polypeptides, such as protamine. Lipopeptides, such as Pam3Cys, are furthermore likewise particularly suitable for use as adjuvants in the pharmaceutical composition of the present invention; c.f. Deres et al., Nature 1989, 342: 561-564.

The present invention further provides a method for the production of a mixture according to the invention, which method comprises the following steps:
a. in vitro transcription of at least one template DNA, which codes for at least one antigen from a tumor,
b. in vitro transcription of at least one template DNA, which codes for at least one immunogenic protein,
c. degradation of the template DNA with suitable means,
d. isolation of the mRNA obtained in steps a. and b by suitable means,
e. mixing of the mRNAs isolated in step d.

Procedures for in vitro transcription have already been described above and are known in the prior art (see for example Maniatis et al., above). Antigens from a tumor and immunogenic proteins which are usable according to the invention have likewise been described above. The degradation of template DNA in step c. may preferably proceed by DNAse treatment, which is well known in the prior art. Isolation of the mRNA may proceed by preferably two or more successive precipitation and/or extraction processes. LiCl precipitation, ethanol/NaCl precipitation and phenol/chloroform extraction may, for example, be considered here. Further methods are well known to the person skilled in the art. Further purification by means of chromatography may then also follow. For mixing, the isolated mRNAs may preferably be present in water, likewise preferably at identical concentrations. Different concentrations may, however, also be selected. Suitable conditions and concentrations under which the mRNAs may advantageously be mixed are likewise well known to the person skilled in the art.

It is furthermore preferred for the isolated and/or mixed mRNA to be present in aqueous solvents, which may comprise PBS, for example. PBS may here depending on suitability be present in different concentrations, for example 1×PBS or 10×PBS. The solvent may furthermore be isotonic saline which may also be buffered with HEPES. It is, however, particularly preferred to use Ringer's lactate solution (from Fresenius). When Ringer's lactate solution was used as buffer, the inventors for the first time achieved 5-times greater effectiveness relative to the prior art.

The invention further provides the use of a mixture according to the invention and/or of a pharmaceutical composition according to the invention for the treatment of neoplastic or tumor diseases, for example melanoma, such as malignant melanoma, skin melanoma, carcinoma, such as colon carcinoma, lung carcinoma, such as small cell lung carcinoma, adenocarcinoma, prostate carcinoma, esophageal carcinoma, breast carcinoma, kidney carcinoma, sarcoma, myeloma, leukemia, in particular acute myeloid leukemia, glioma, lymphomas, and blastomas. In tumor diseases, the mRNA according to the invention which codes for a tumor antigen preferably codes for a tumor-specific surface antigen (TSSA).

The invention likewise further provides the use of a mixture according to the invention for the production of a medicament for the treatment of neoplastic or tumor diseases, for example melanoma, such as malignant melanoma, skin melanoma, carcinoma, such as colon carcinoma, lung carcinoma, such as small cell lung carcinoma, adenocarcinoma, prostate carcinoma, esophageal carcinoma, breast carcinoma, kidney carcinoma, sarcoma, myeloma, leukemia, in particular acute myeloid leukemia, glioma, lymphomas, and blastomas. In tumor diseases, the mRNA according to the invention which codes for a tumor antigen preferably codes for a tumor-specific surface antigen (TSSA). The term “medicament” and the term “pharmaceutical composition” should be regarded as synonymous according to the invention.

The present invention is further illustrated below with reference to Figures and Examples, without there being any intention in so doing to restrict the subject matter of the present invention thereto.

### FIGURES

In the following FIGS. 1 to 13, which show RNA nucleic acid sequences, the start codon and optionally also the stop codon are in each case shown in bold letters. The grey highlighting indicates those sequence portions which relate to the untranslated region (UTR) of the human alpha-globin gene, which stabilizes the mRNA and furthermore increases mRNA translation.

FIG. 1 shows the melan A-αg-A₇₀ RNA nucleic acid sequence

FIG. 2 shows the tyrosinase-αgA₇₀ RNA nucleic acid sequence

FIG. 3 shows the MAGE A1-αgA₇₀ RNA nucleic acid sequence

FIG. 4 shows the MAGE A6-αGA₇₀ RNA nucleic acid sequence

FIG. 5 shows the survivin-αgA₇₀ RNA nucleic acid sequence

FIG. 6 shows the Her-2/Neu-αgA₇₀ RNA nucleic acid sequence

FIG. 7 shows the CEA-αgA₇₀ RNA nucleic acid sequence

FIG. 8 shows the mucin1-αgA₇₀ RNA nucleic acid sequence

FIG. 9 shows the GP100-αgA70 RNA nucleic acid sequence

FIG. 10 shows the □g-FLUWT-αgA₇₀ RNA nucleic acid sequence. This comprises the nucleic acid sequence of a variant, containing a point mutation, of the influenza A/Hong Kong/1/68 matrix protein. Accession number AF348197

FIG. 11 shows the βg-FLUGC rich-αgA₇₀ RNA nucleic acid sequence. This comprises the GC-enriched nucleic acid sequence which codes for a protein which is identical to the influenza A/PR/8/34 matrix protein, accession number V01099.

FIG. 12 shows the HBs-αgA₇₀ RNA nucleic acid sequence

The above-stated RNA-sequences shown in the Figures contain not only the coding domain but also further sequences at the 5′ and 3′ termini. As shown in the Figures, Kozak sequences or ribosome binding sequences may, for example, be present at the 3′ terminus. A poly-A sequence, for example from a globin gene (α or β), may be present at the 5′ terminus. In the above-stated sequences, the untranslated α-globin sequence was in each case attached flanking on the 5′ and 3′ end of the coding domain of the stated genes.

FIG. 13 shows the effect of using different buffers on mRNA expression. To this end, various injection batches (containing mRNA, which codes for luciferase, and in each case different buffers) were injected into the ear of different mice and the expression rate was determined by measuring luciferase activity by means of light emission. The test method is explained in detail in Example 2 below. Measurements were made every 15 seconds for 45 seconds. Accordingly the values on the x-axis in FIG. 13 are in each case shown in three columns for each of the buffers. The y-axis shows the expression rate in NLU/sec mouse. As can be seen, the expression rate of the injection batch containing Ringer's lactate solution as buffer is very much higher than with the other buffer systems used.

FIG. 14: FIG. 14 shows the course of the clinical trial, with two different experimental protocols (I (upper table), II (lower table)). W (=weeks). In the first experimental protocol, injections were initially provided every two weeks and subsequently at 4 week intervals (X indicates administration). In the second experimental protocol, two injections were provided in the first two weeks and then every 4 weeks. The injections (in each case identical in the two experimental protocols) were administered to the patients of the two experimental protocols in each case in parallel intradermally in the left and right leg. In both experimental protocols, the injected solutions contained an RNA mixture according to the invention composed of identical absolute quantities of tumor antigen RNA (survivin, CEA, mucin-1, Her-2/Neu, MAGE-A1) and viral antigen RNA (influenza matrix GC rich, HBs). The administered tumor antigen RNAs corresponded to the sequences shown in FIGS. 3 (MAGE-A1), 5 (survivin), 6 (HER2 Neu) and 7 (CEA) or 8 (mucin1). The administered viral antigen HBs corresponded to the sequence in FIG. 12 and the further viral antigen of FLU-GC rich sequence shown in FIG. 11. The latter is the only G/C optimized sequence present in the administered mixture. In total, 200 μg of RNA of the above-stated antigens were dissolved in 300 μl of Ringer's lactate solution. Approx. 100 μl of the solution (per injection) were administered into the patients' left or right leg. The rest of the solution remained in the syringe. In each case one day after administration of the RNA mixture, GM-CSF was administered to the patients, likewise intradermally in the leg.

The image of the agarose gels shows the bands of the eight different RNA antigens in the mixture according to the invention.

FIG. 15: FIG. 15 shows experimental protocol 1 (arm 1) with a total of treated 16 patients suffering from malignant diseases (RCC, renal cell carcinoma, ovarian cell carcinoma or breast cancer) and (arm 2) 11 patients with RCC or colorectal carcinoma. The tumors stated are in each case the primary tumors. However, the patients also have metastasizing secondary tumors. The patients were subjected to computerized tomography (CT scan). The patients' status was evaluated in this manner (S: stable, P: progressive or R: regressive).

FIG. 16: FIG. 16 shows intracellular cytokine staining. The cellular immune response was evaluated by FACS analysis. Blood cells were harvested and frozen after different periods of time (T). After T 8, all the samples are evaluated by analyzing IFN-gamma secretion and regarding this as an activation marker for a CD4 T cell response or CD8 T cell response. The administered RNA cocktail according to the invention was separately investigated for stimulators (flu and HBs) or tumor antigens (the remainder).

FIG. 17: The plots of FIG. 17 show the course of the measured cell counts for CD4 or CD8 cells in three different patients. The plots show the correlation between the biochemical findings (see Example 4) and the clinical results in the patient. In patient 1, clinical stability was found on every occasion on which CT scans were recorded (before treatment and in each case quarterly thereafter) (SSSS). Patient 2 (Pat2), in contrast, exhibited a progressive course of the disease at the time of the final CT scan (SSSP). A progressive course of the disease correlates with lower CD4 or CD8 cell titers. The plot shows the percentage of gamma-interferon positive cells per patient.

FIG. 18: FIG. 18 shows images from CT scans, the patient's lungs being shown before (left hand side) and after (right hand side) vaccination with the RNA mixture according to the invention. As indicated by the size of this secondary tumor in the lung (see arrows), there has been a significant reduction in its size in the patient after 6 months of treatment.

FIGS. 19 to 81 show RNA sequences of various genes, in each case designated, which have been sequence optimized with regard to their respective G/C content. Sequence optimization was carried out in accordance with the method described in published patent application WO 2002/098443, i.e. the sequences exhibit a maximum G/C content, without there being any modification of the protein coded in each case thereby, so stabilizing the RNA. All the sequences of FIGS. 19 to 81 (which only show the coding domain) may be present in an RNA mixture according to the invention, preferably with modification at the 3′ and/or 5′ terminus (for example with addition of a Kozak sequence or a poly-tail or a ribosome binding site).

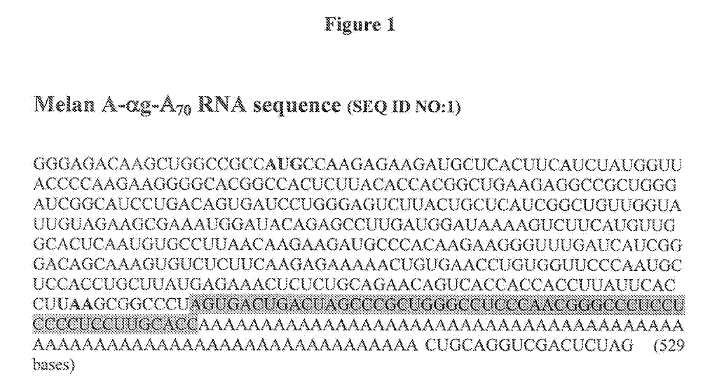

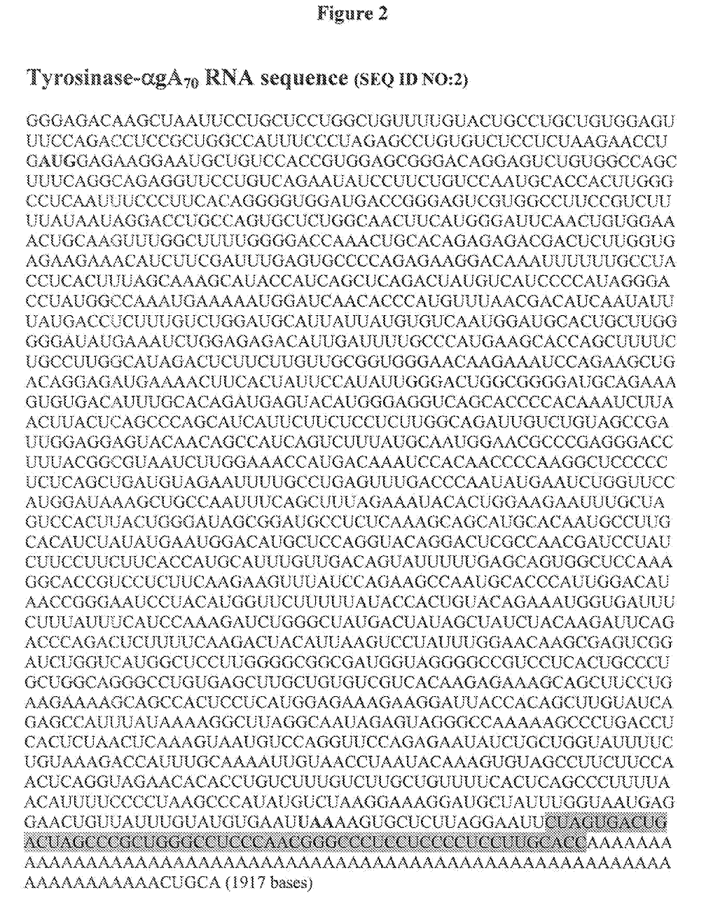

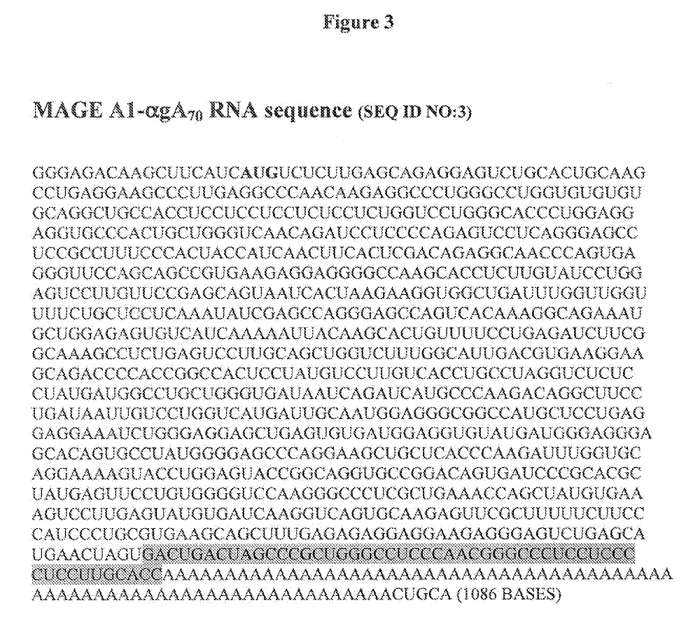

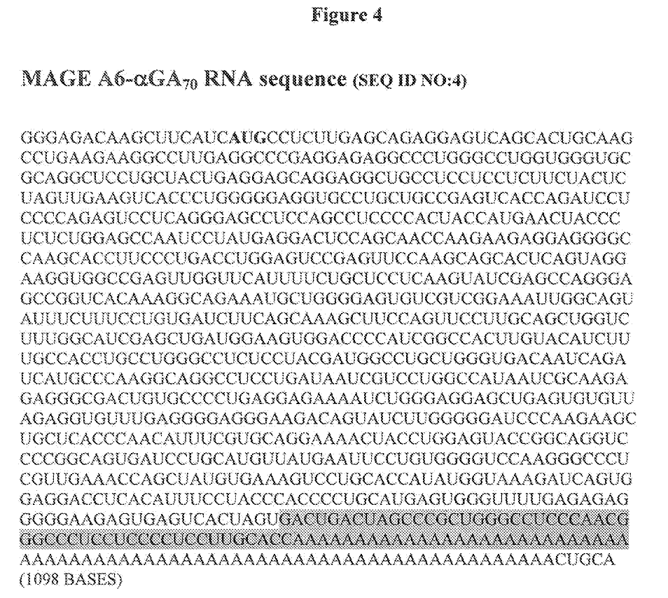

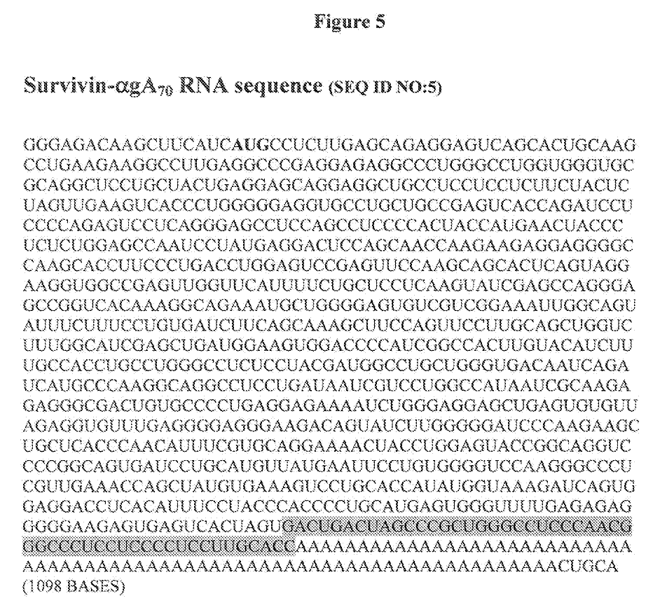

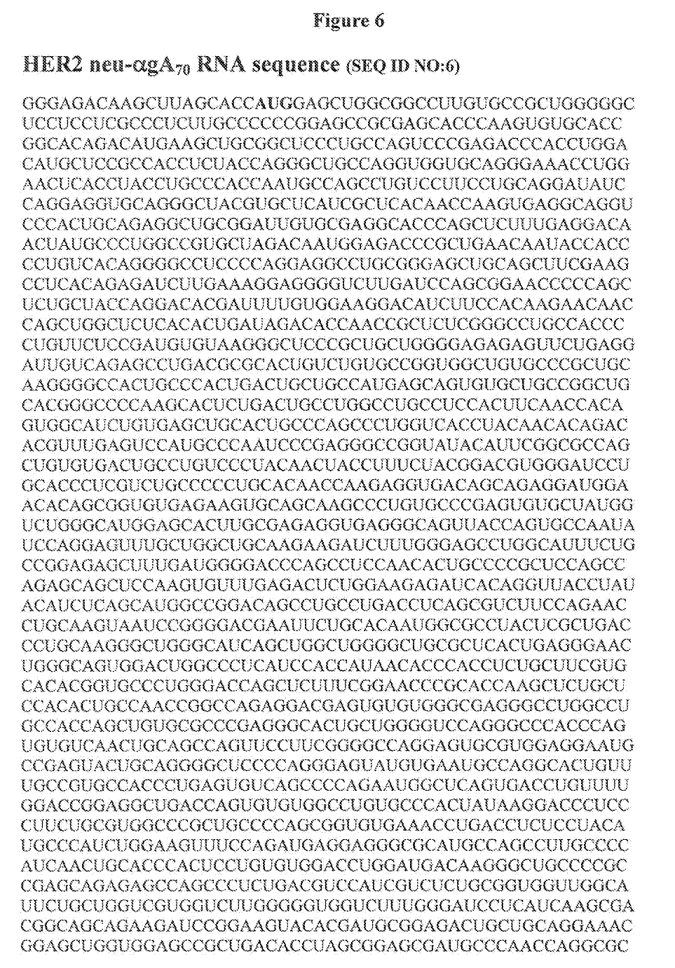

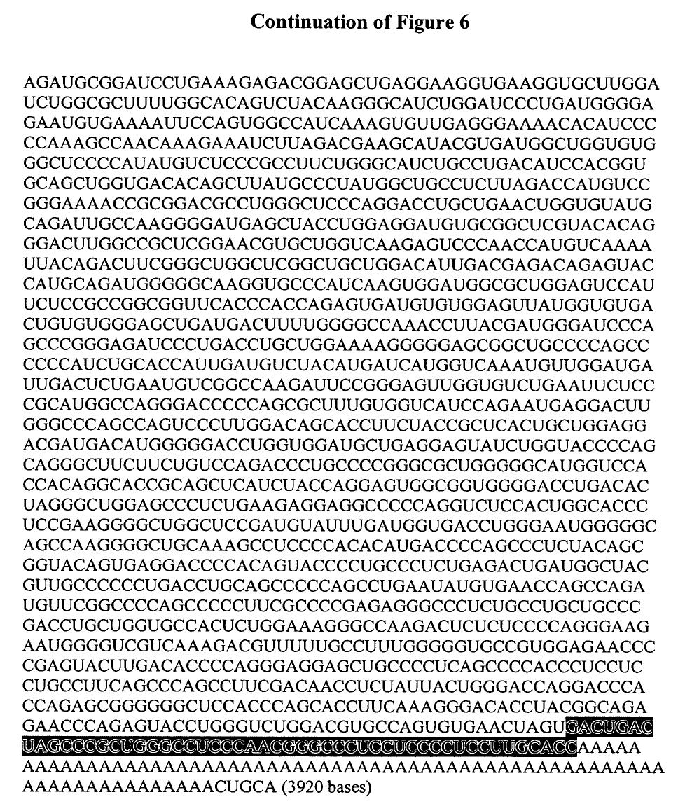

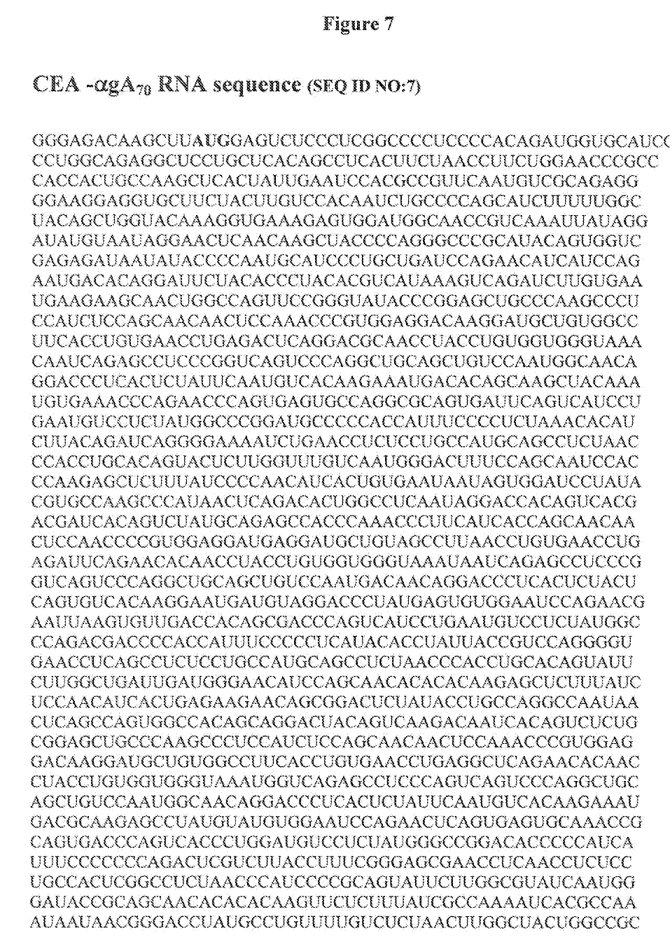

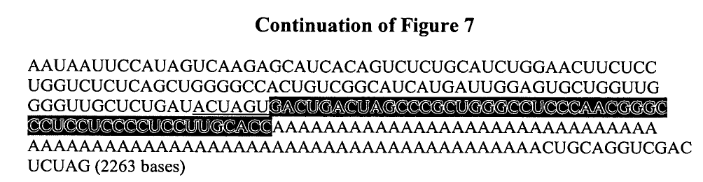

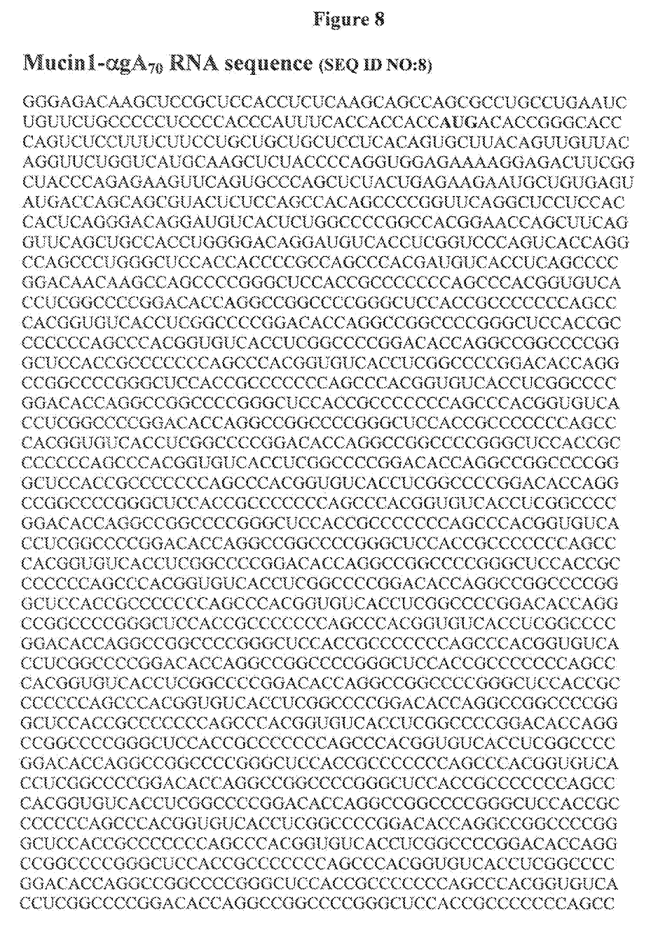

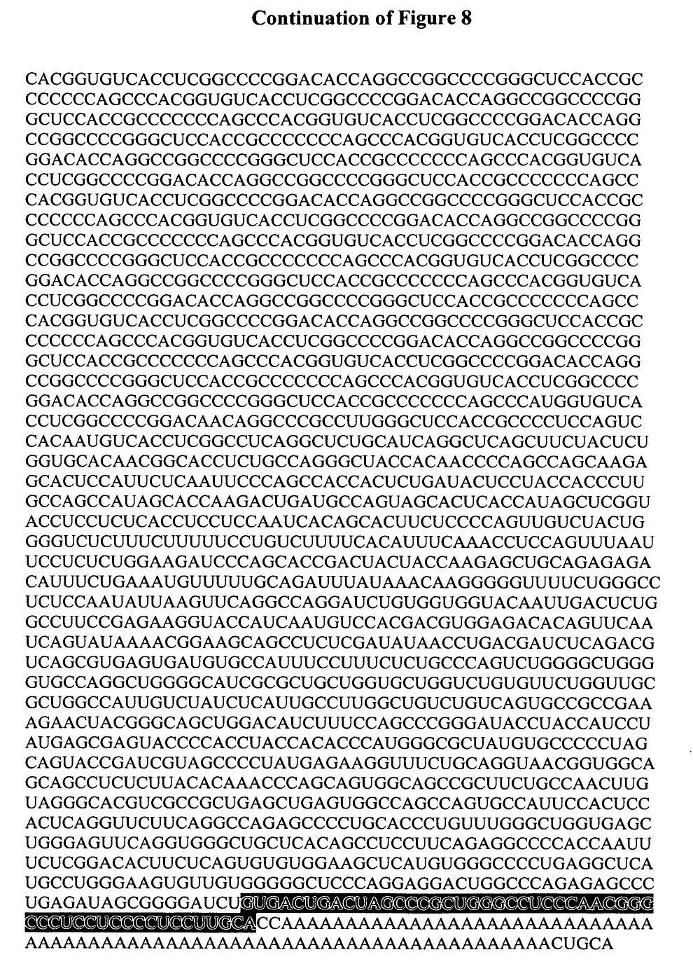

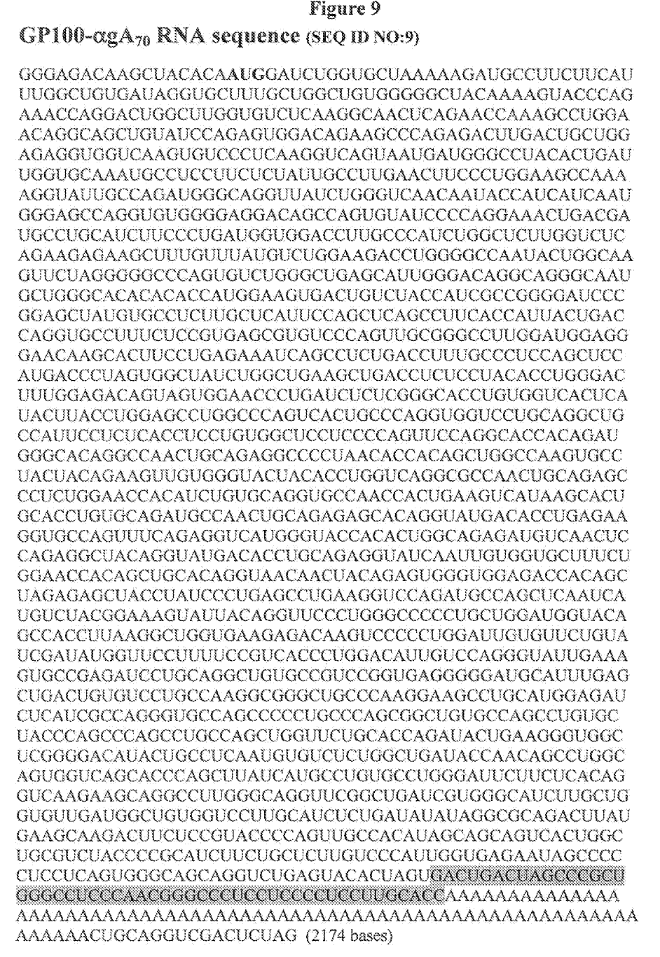

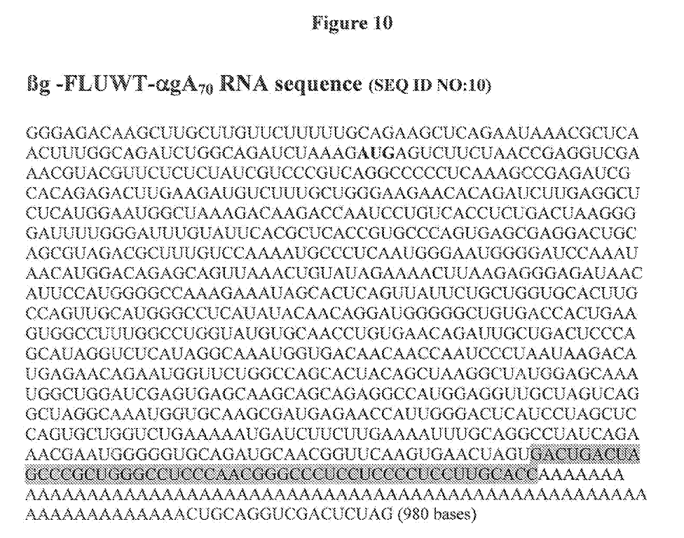

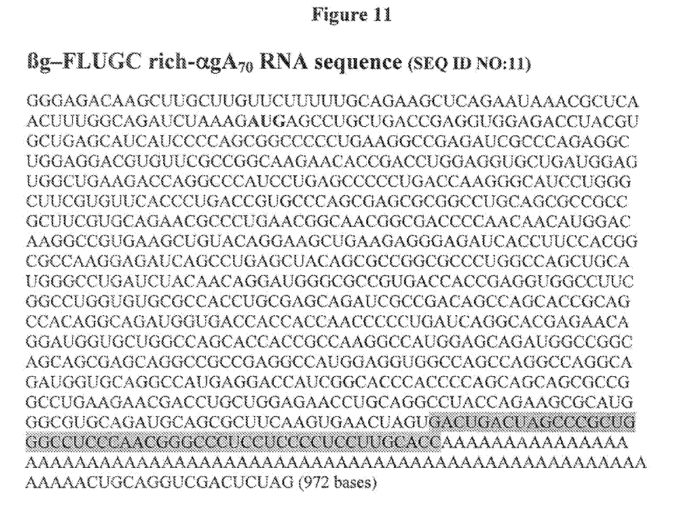

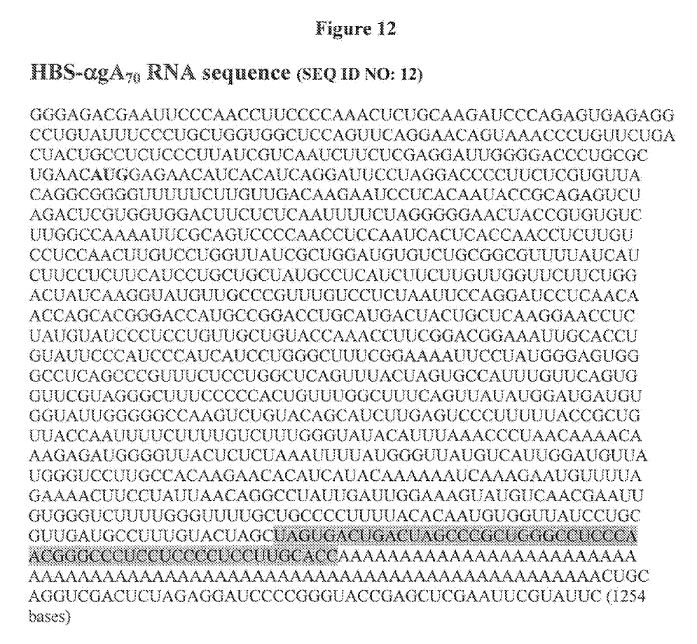

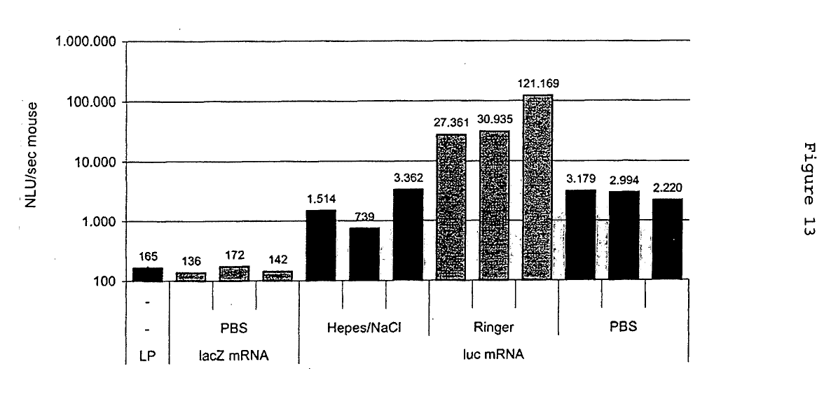

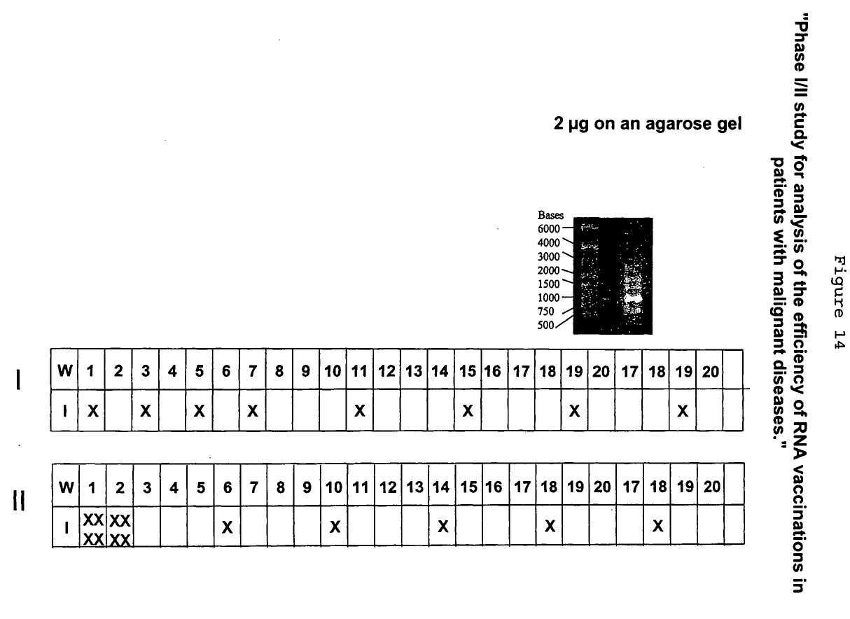

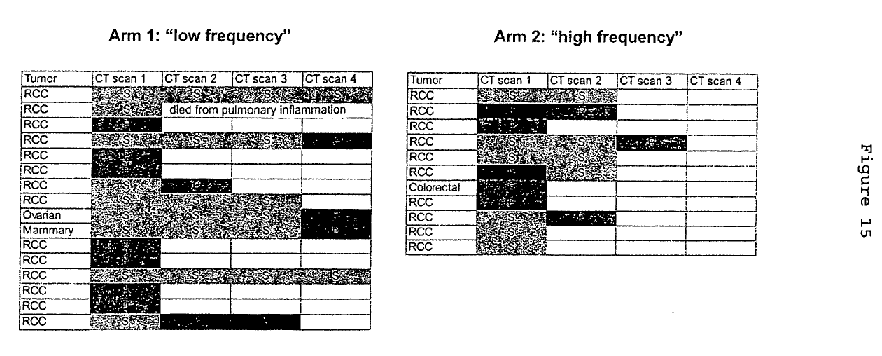

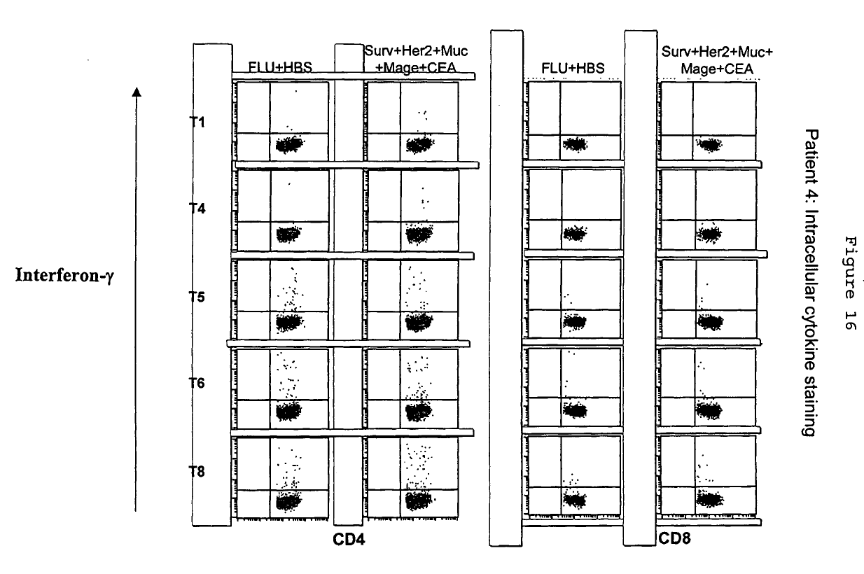

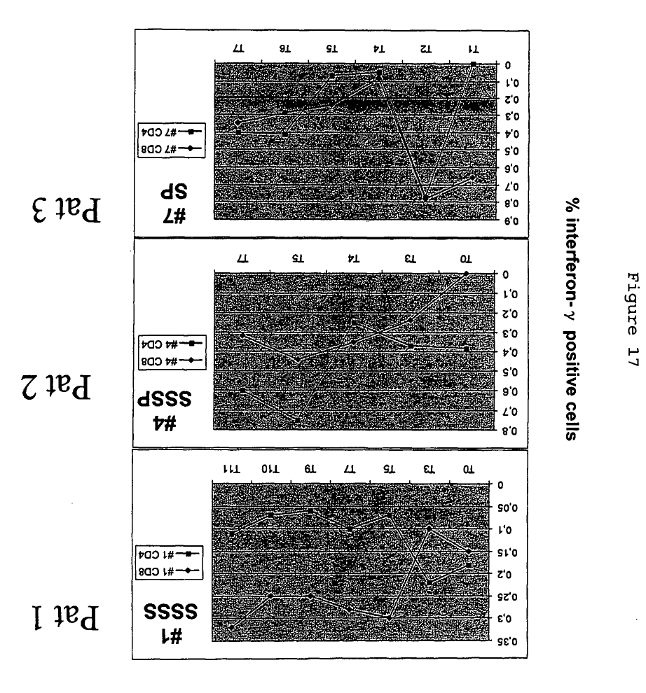

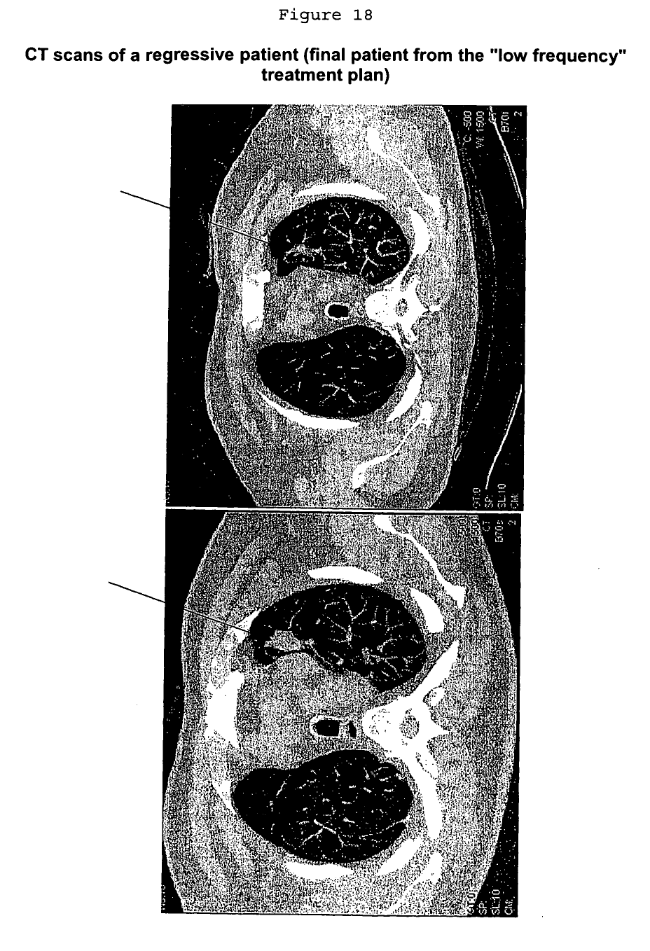

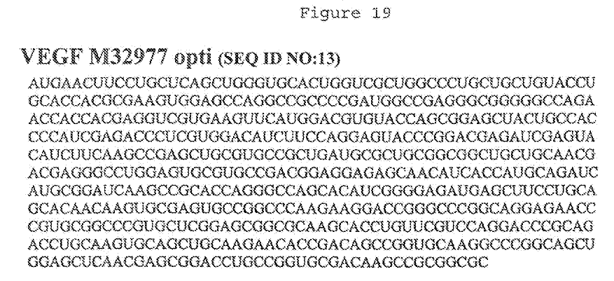

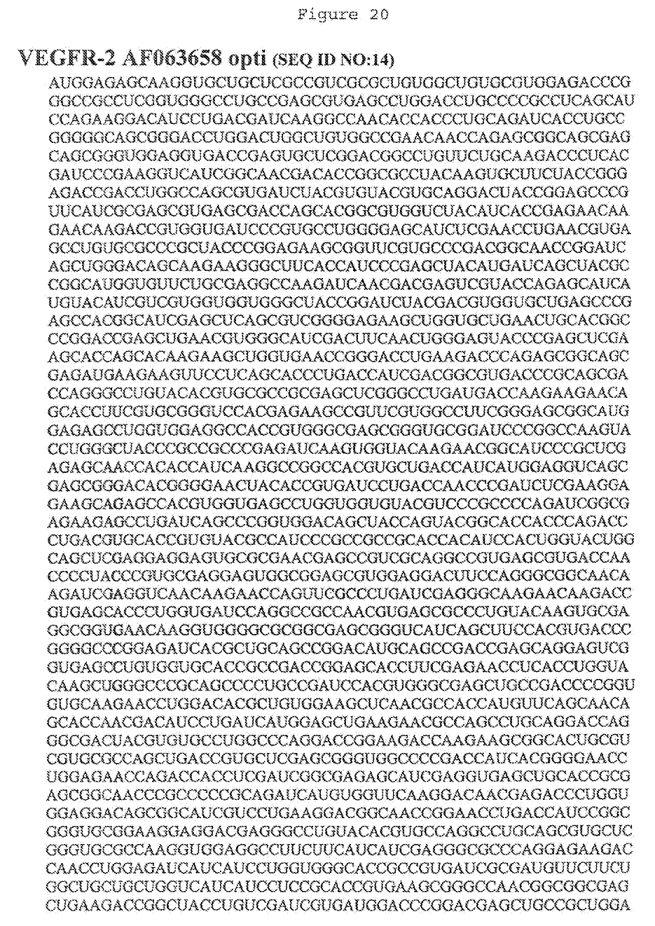

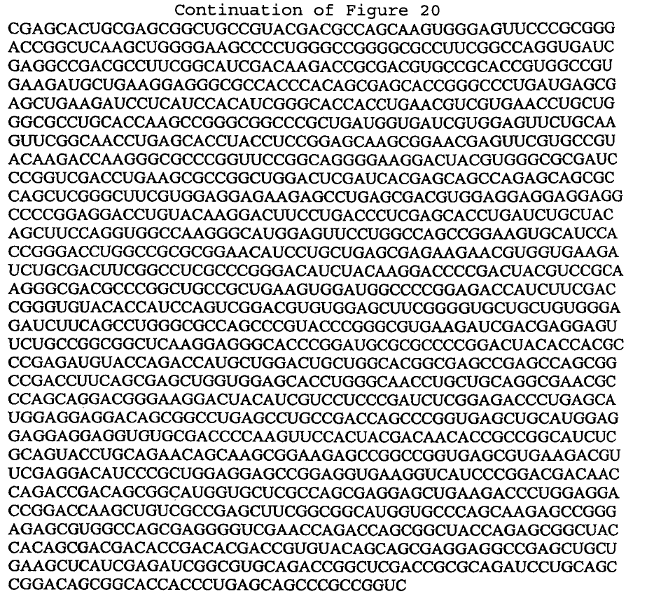

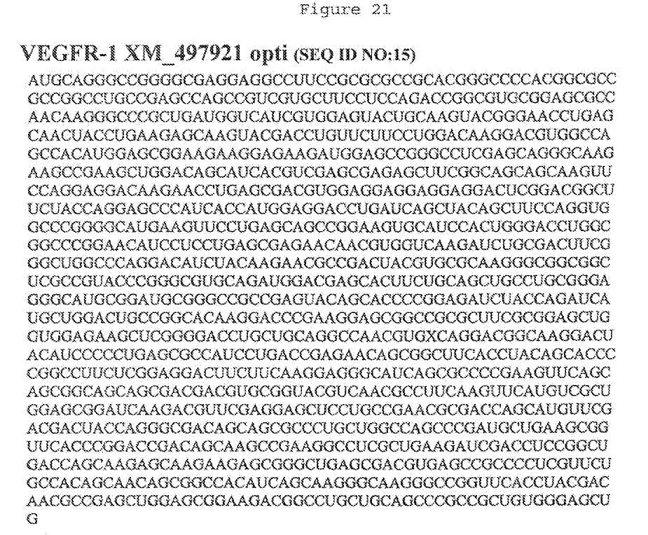

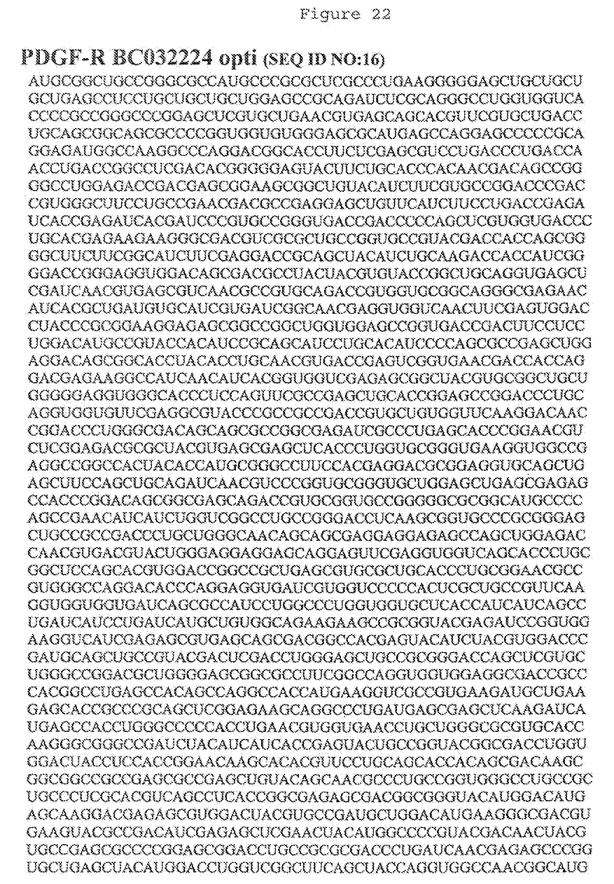

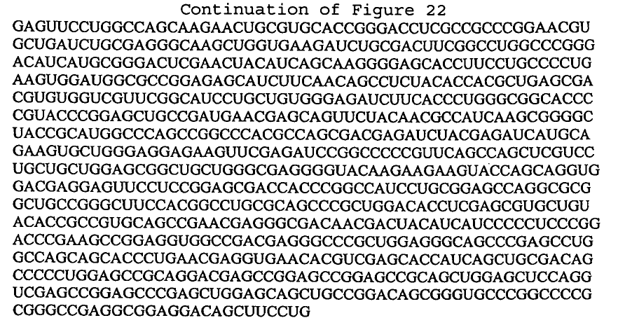

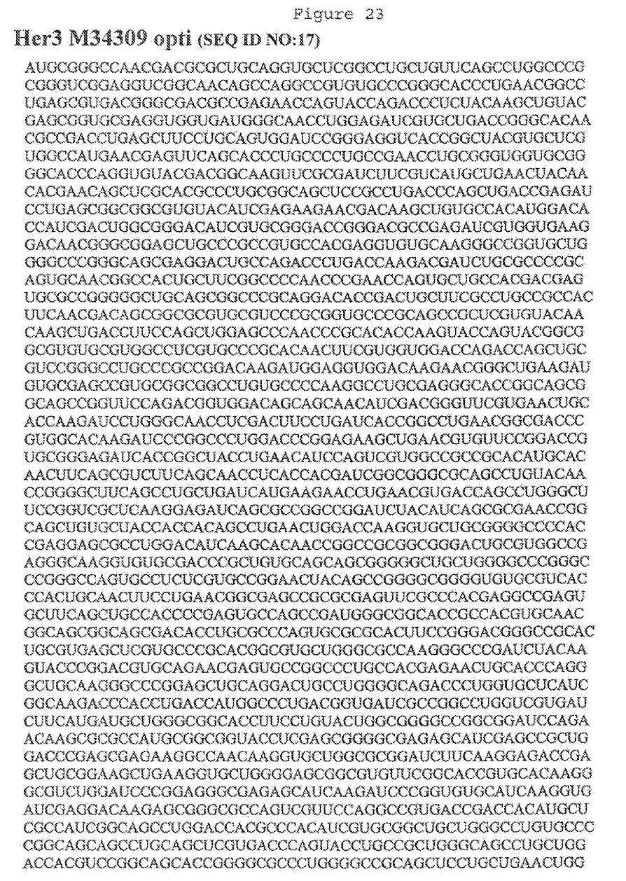

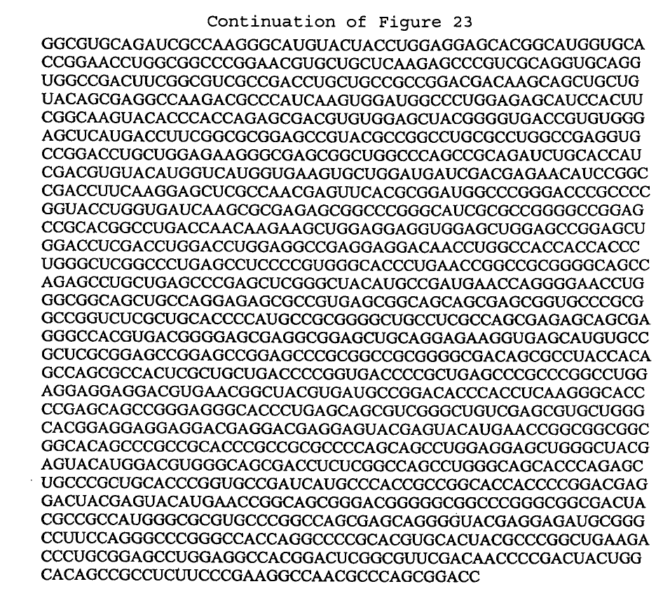

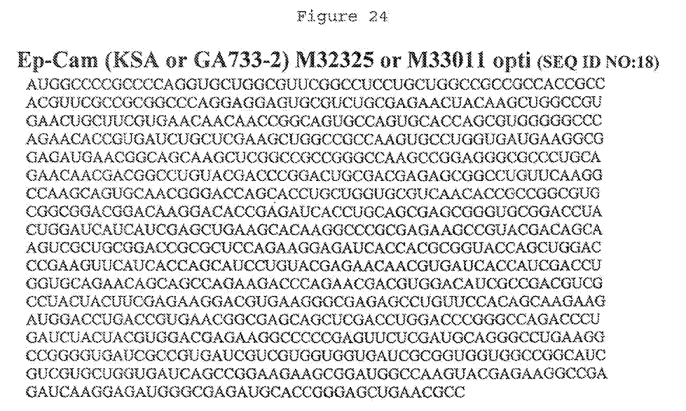

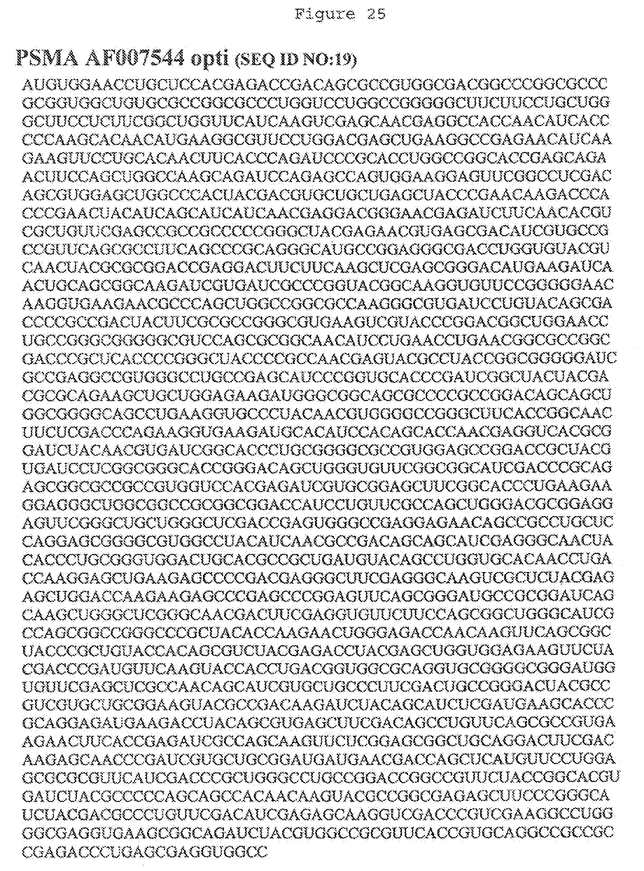

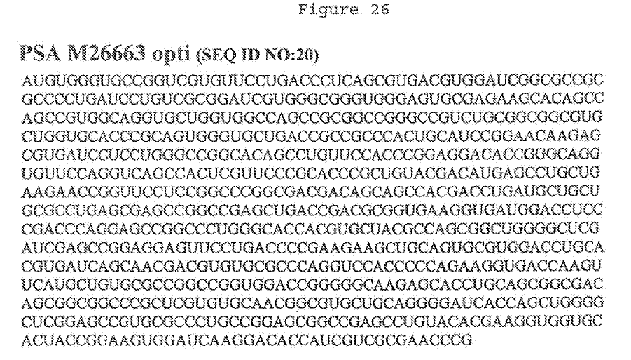

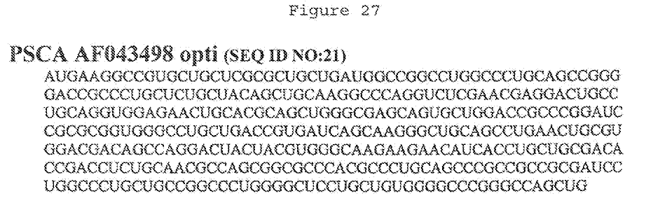

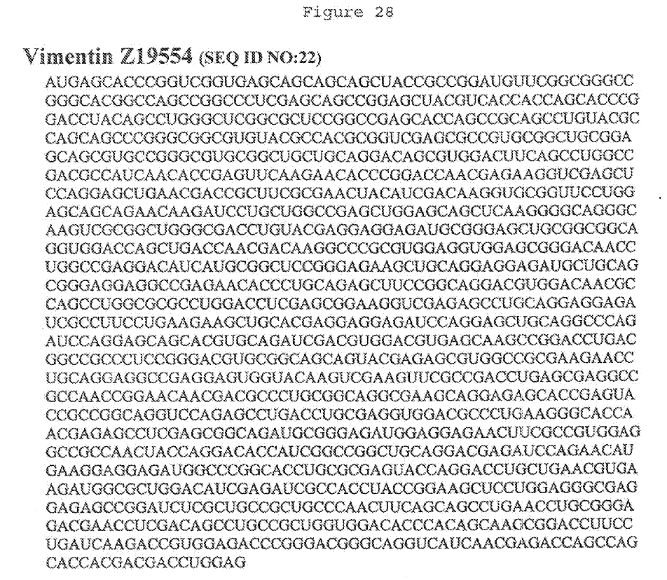

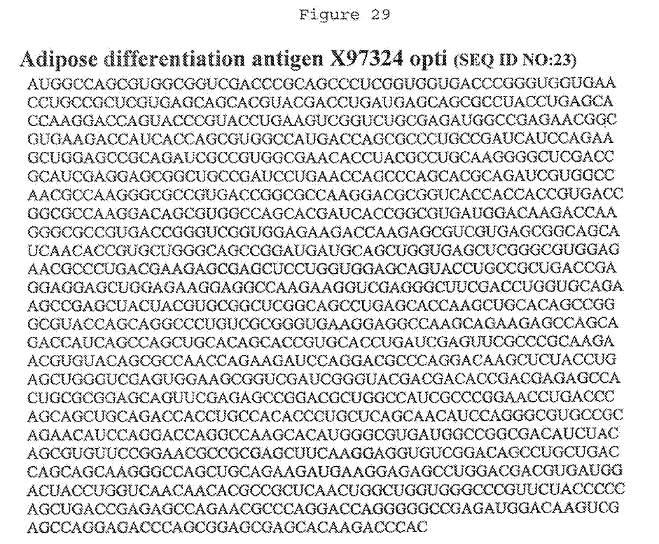

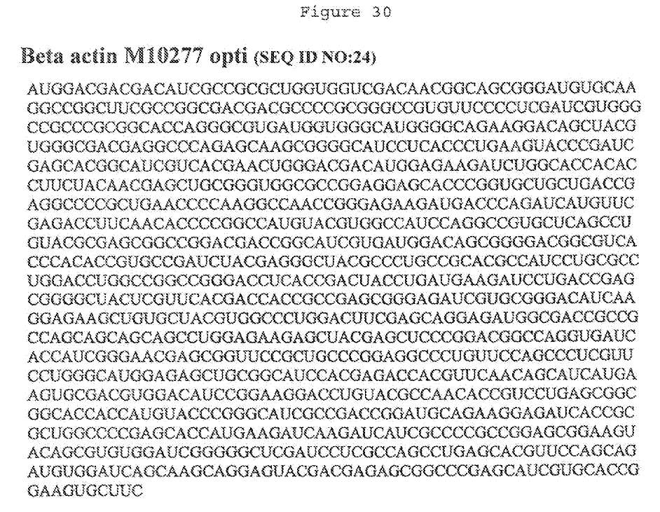

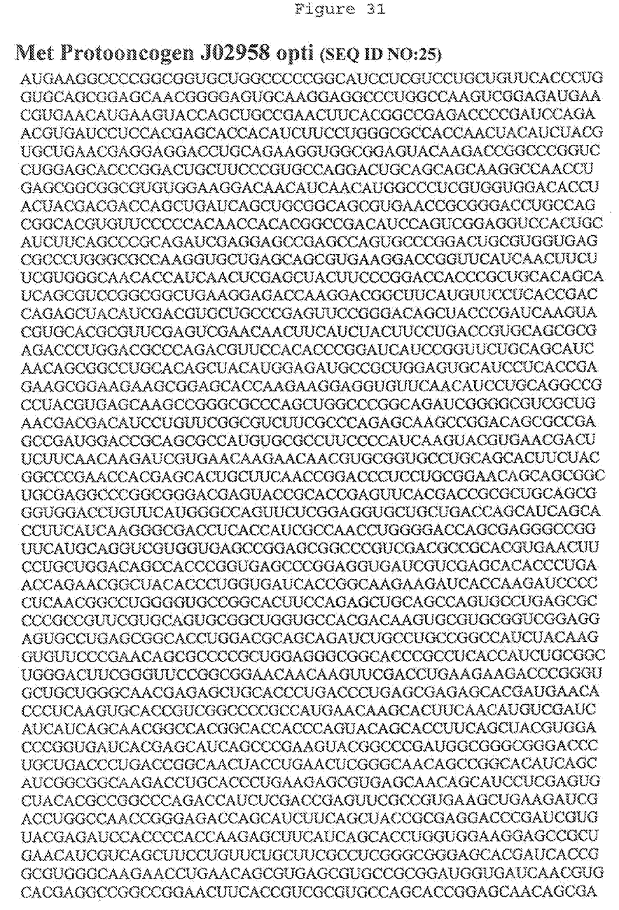

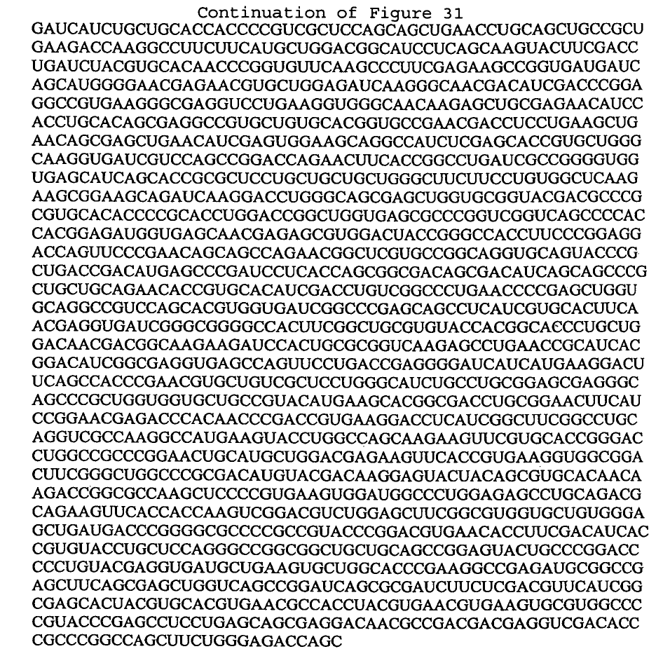

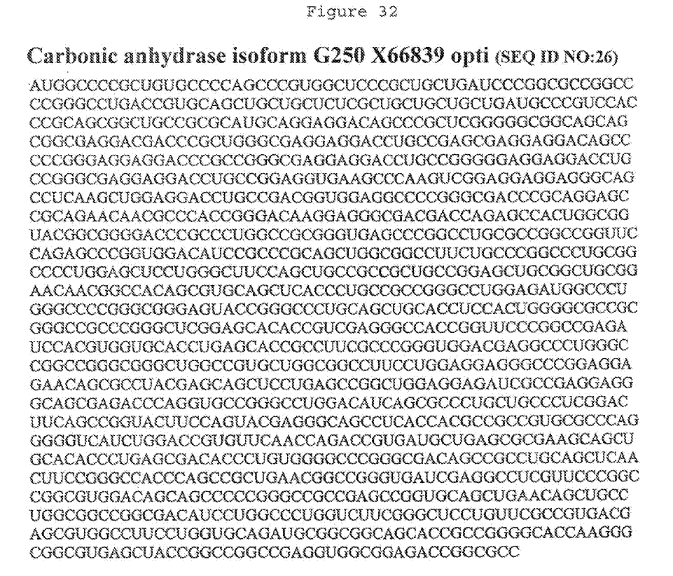

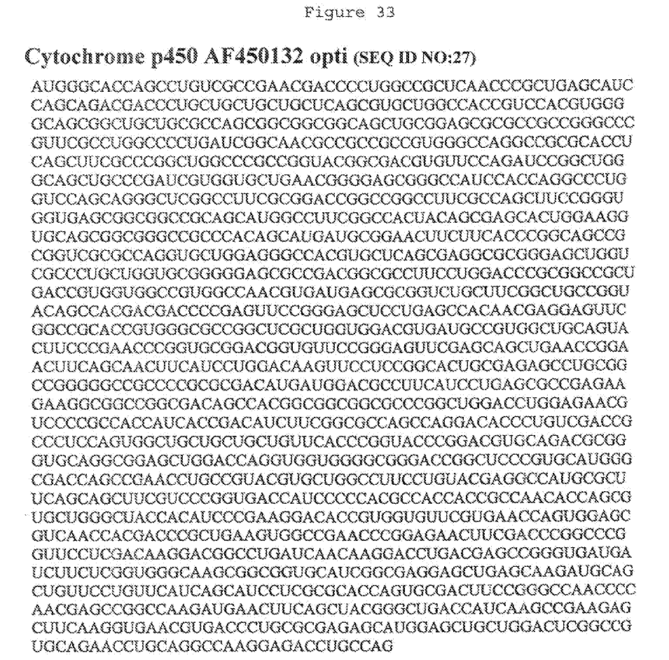

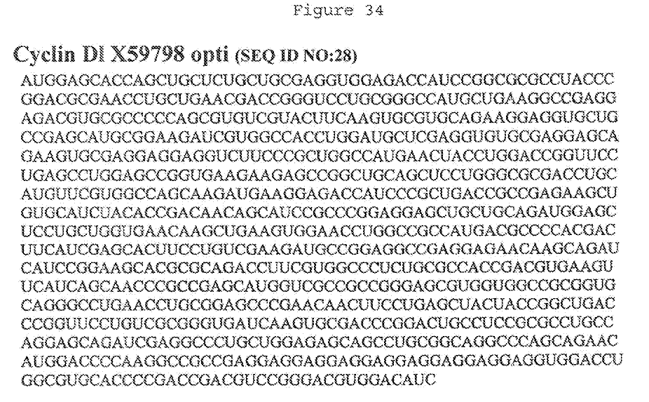

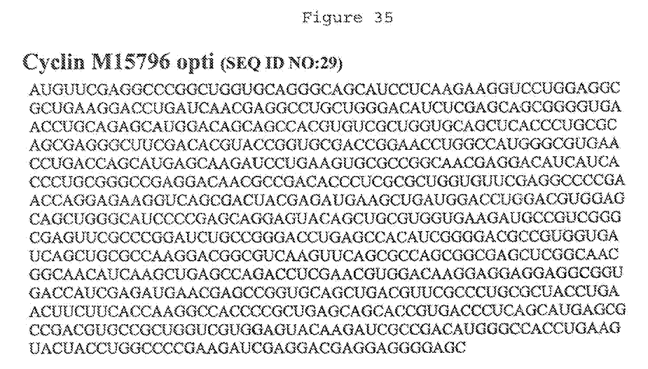

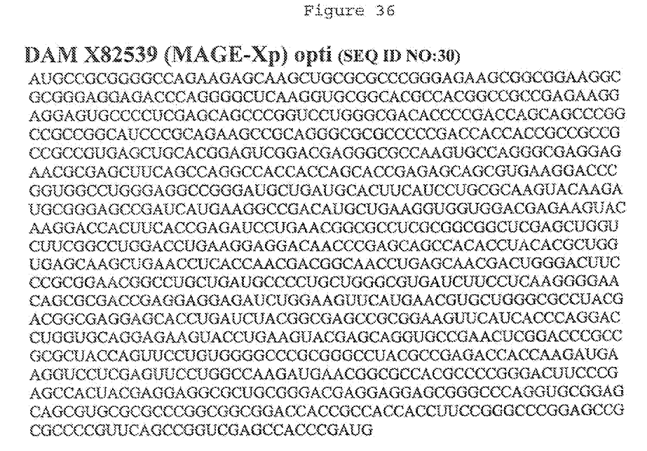

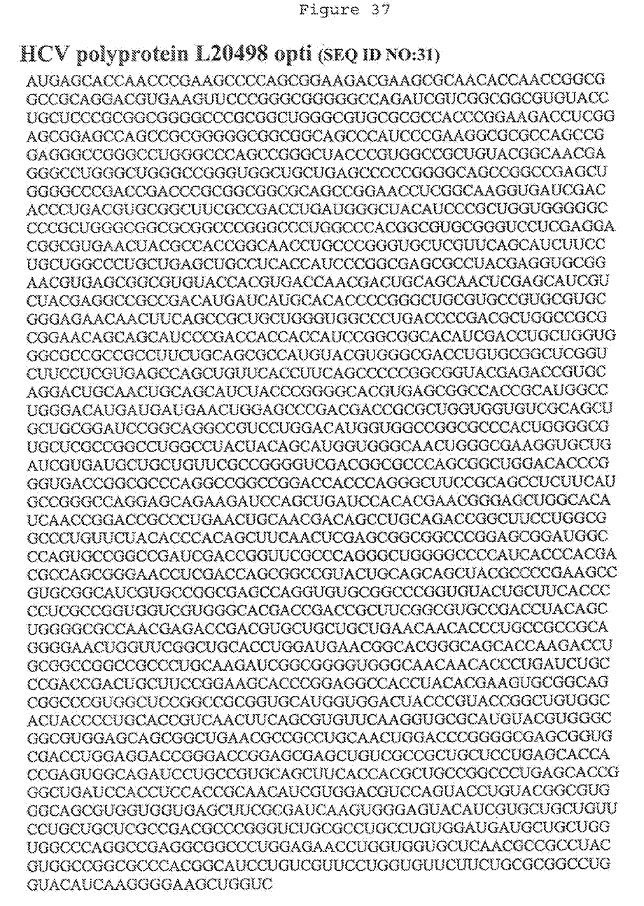

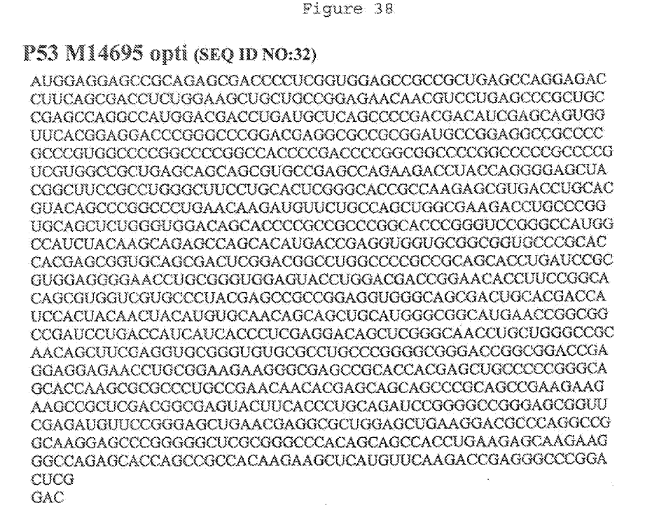

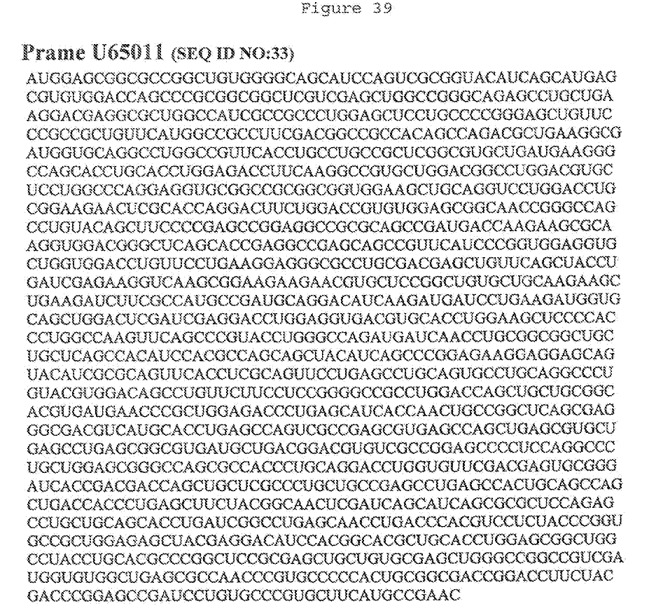

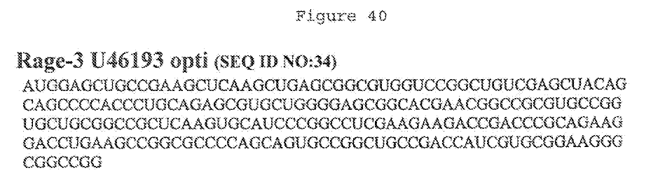

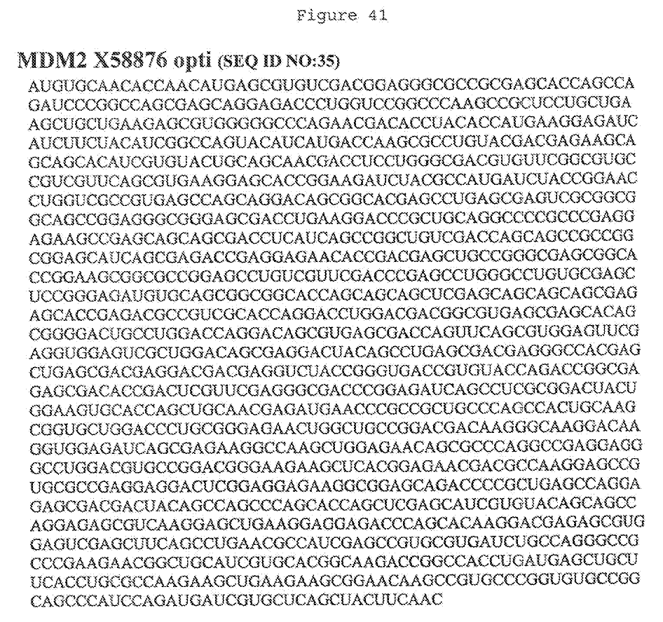

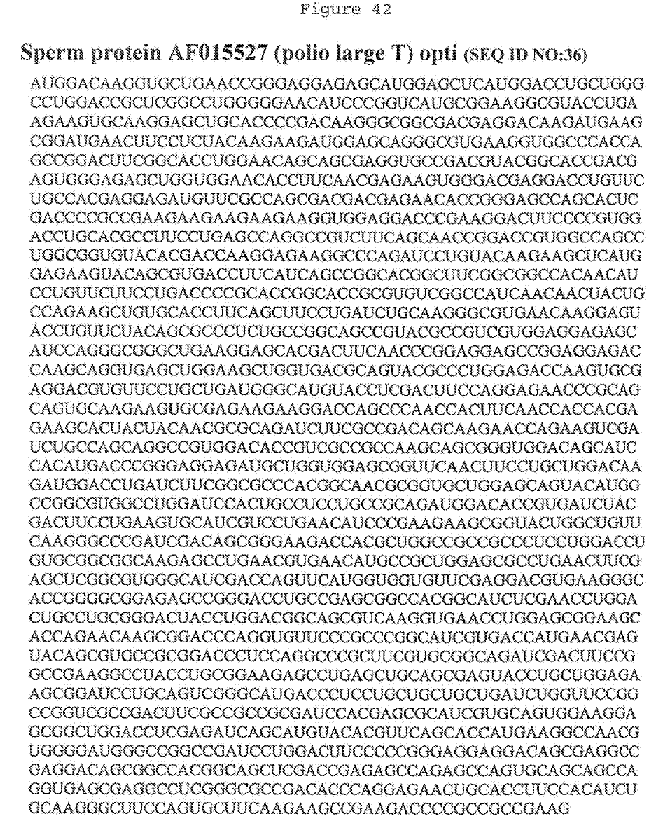

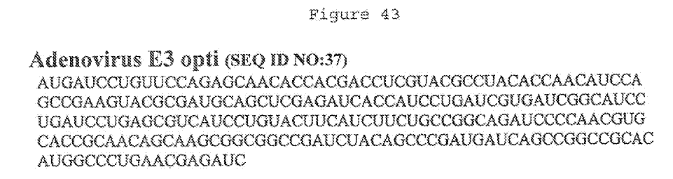

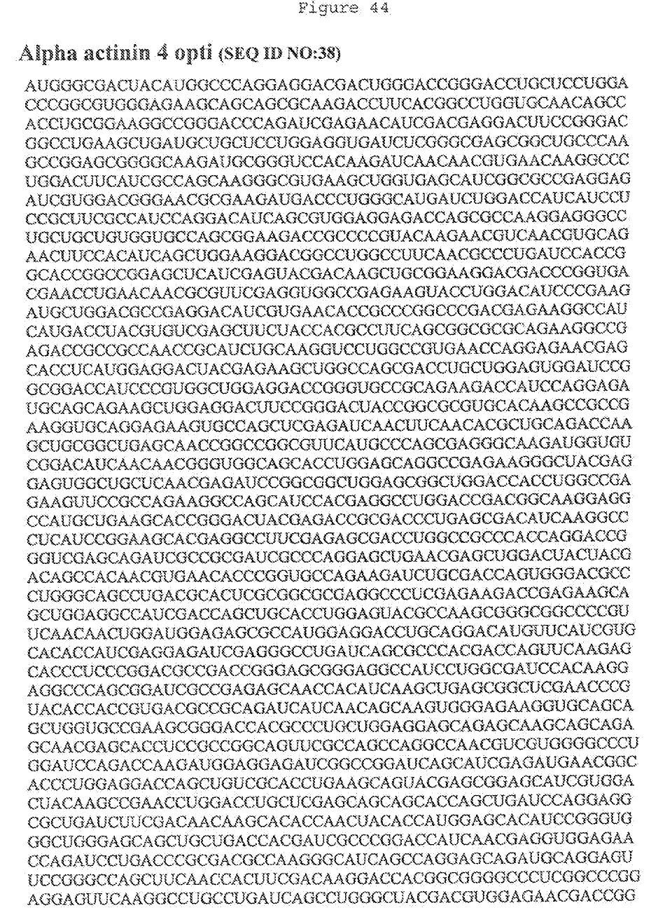

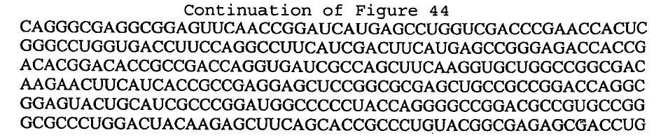

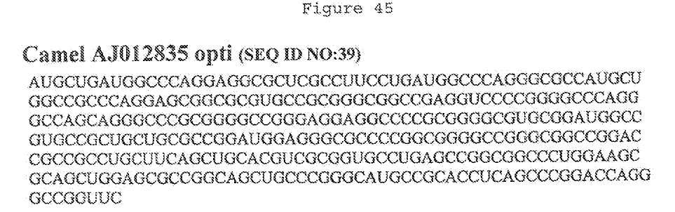

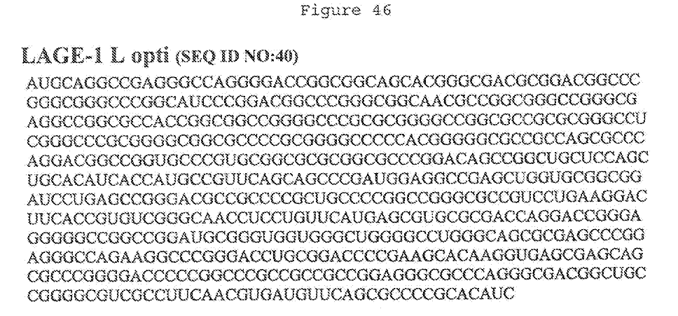

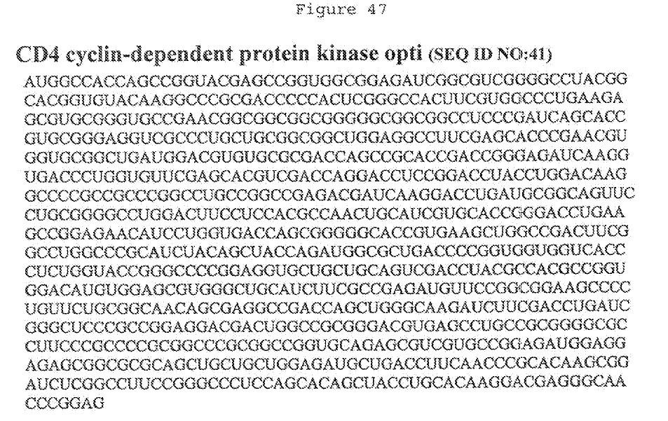

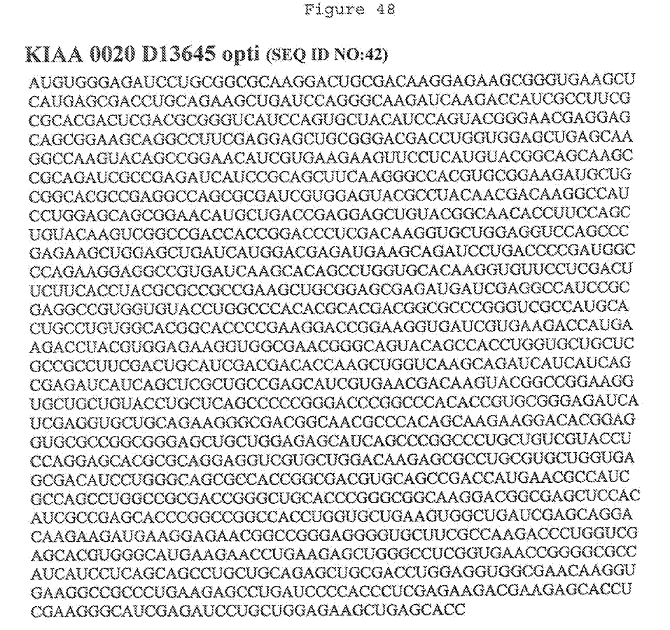

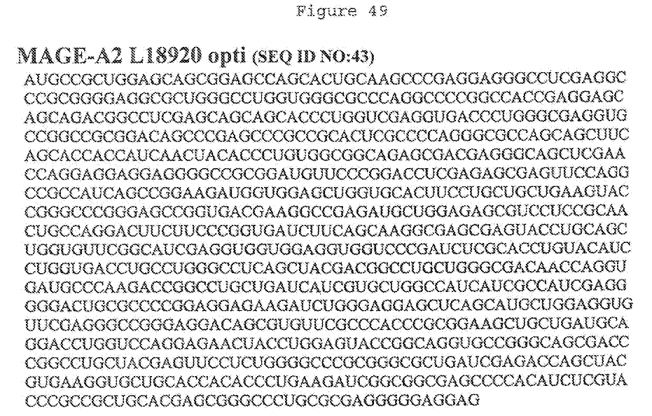

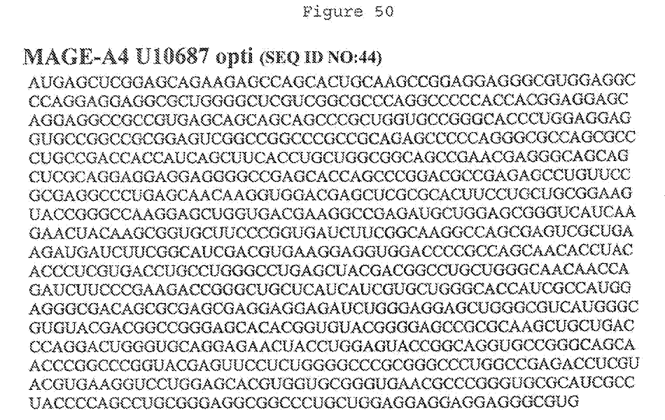

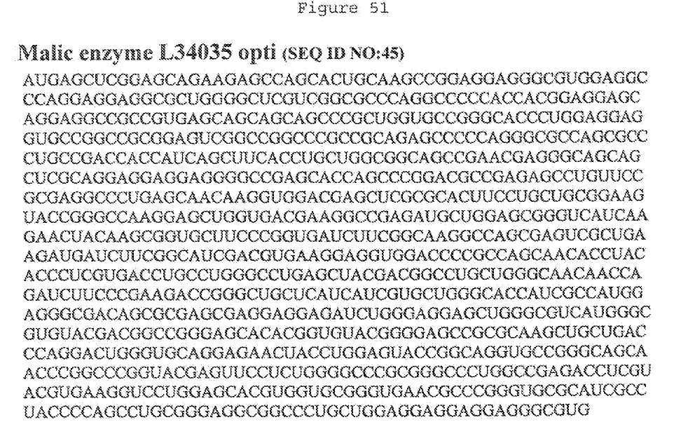

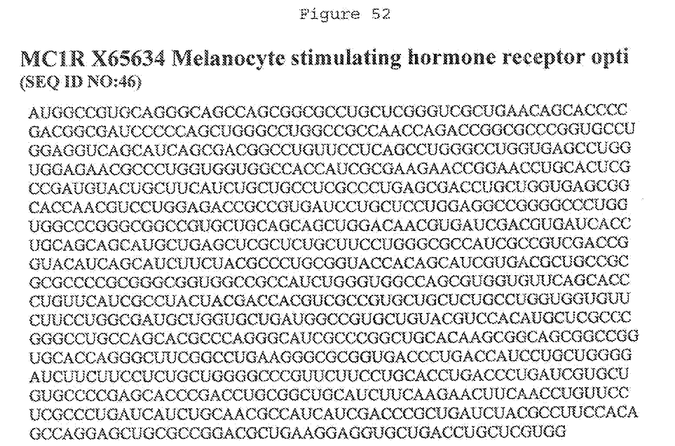

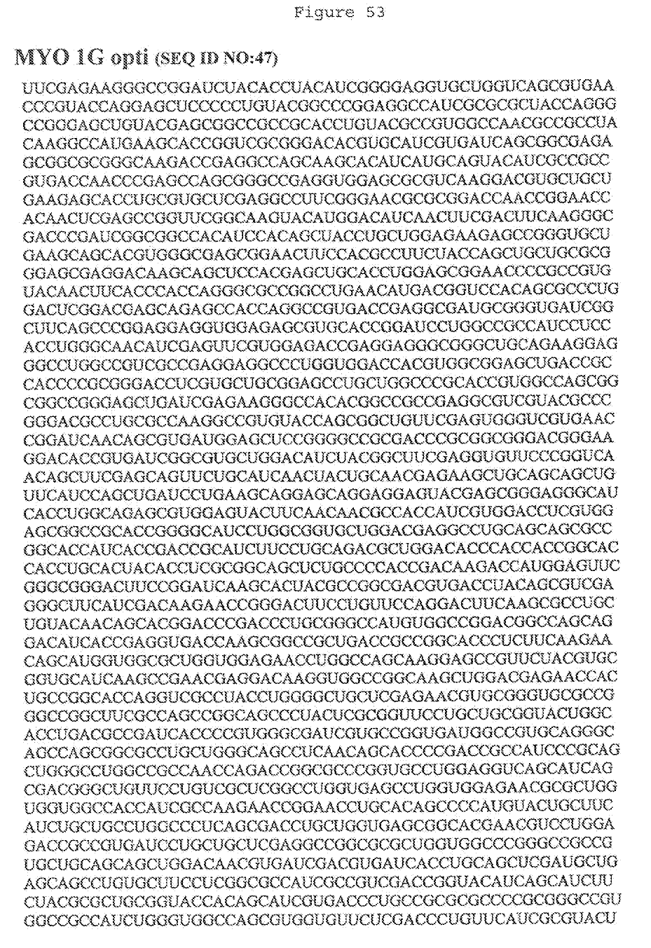

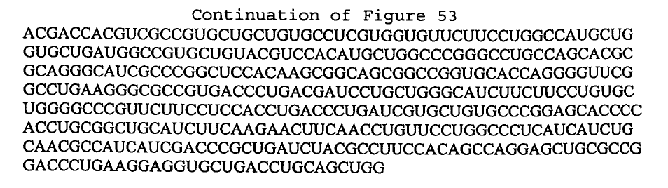

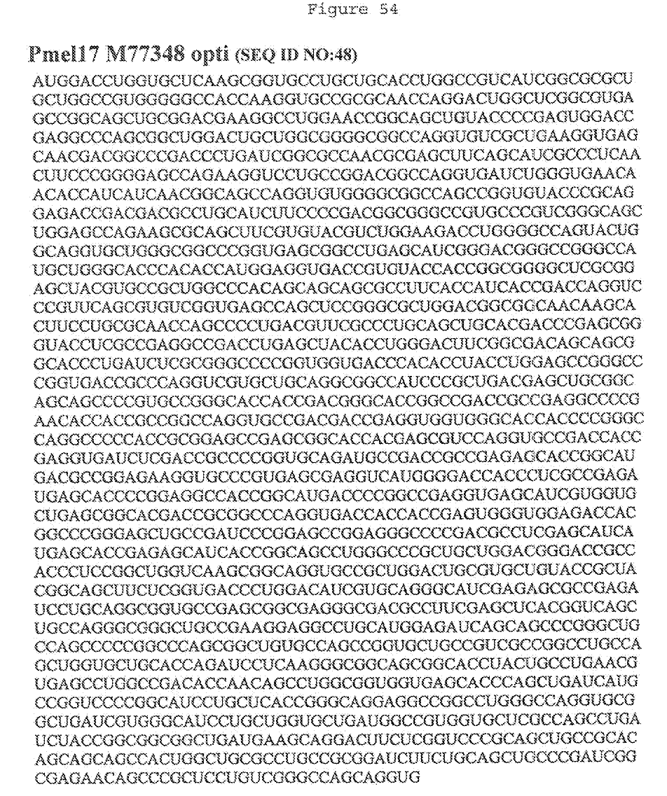

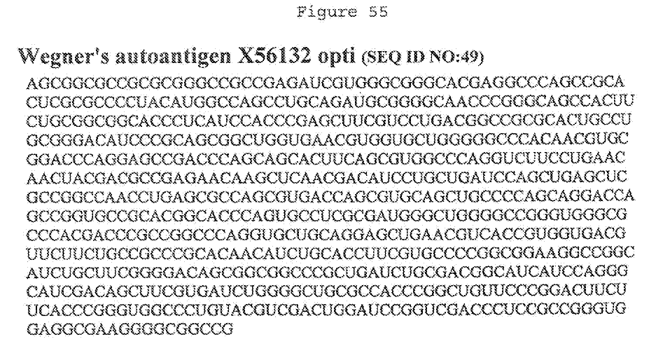

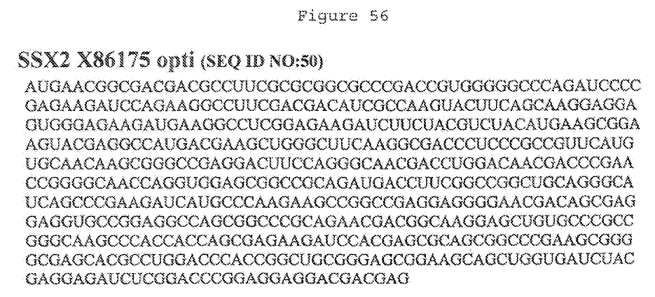

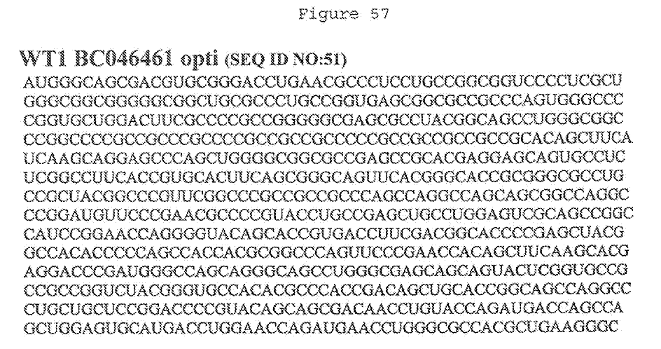

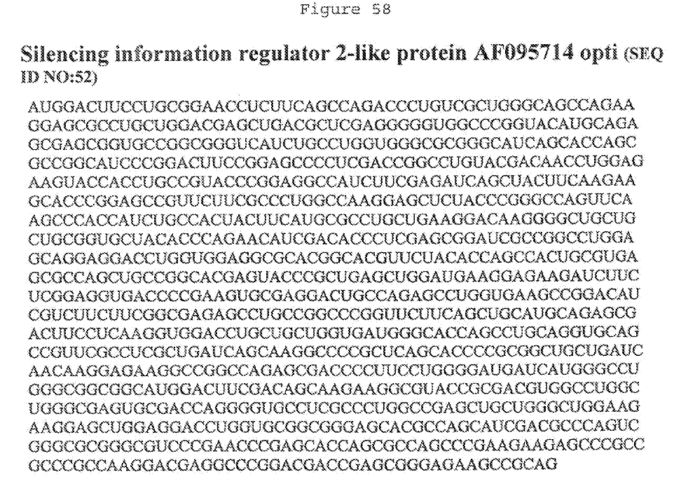

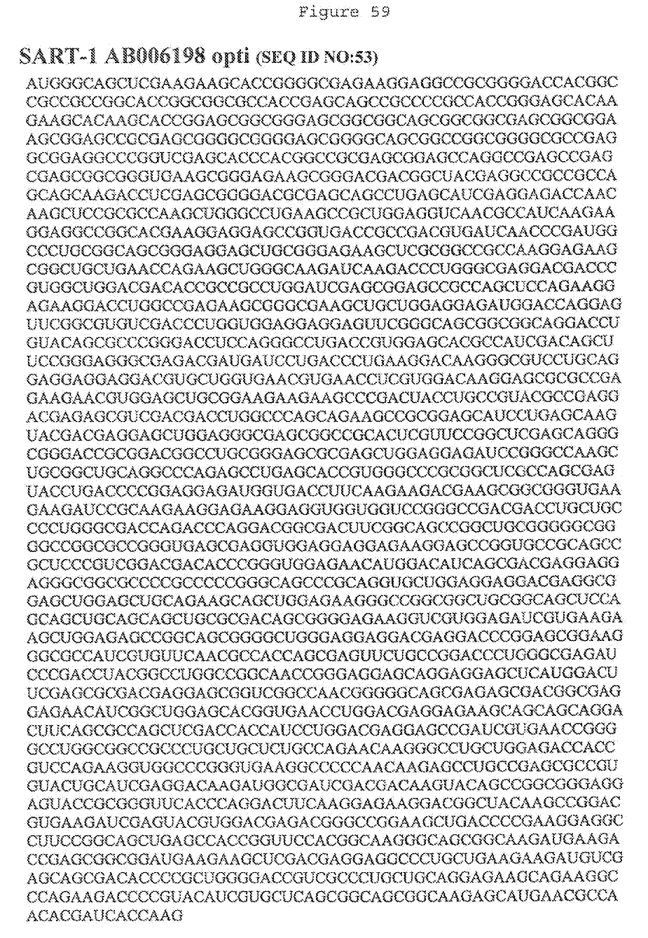

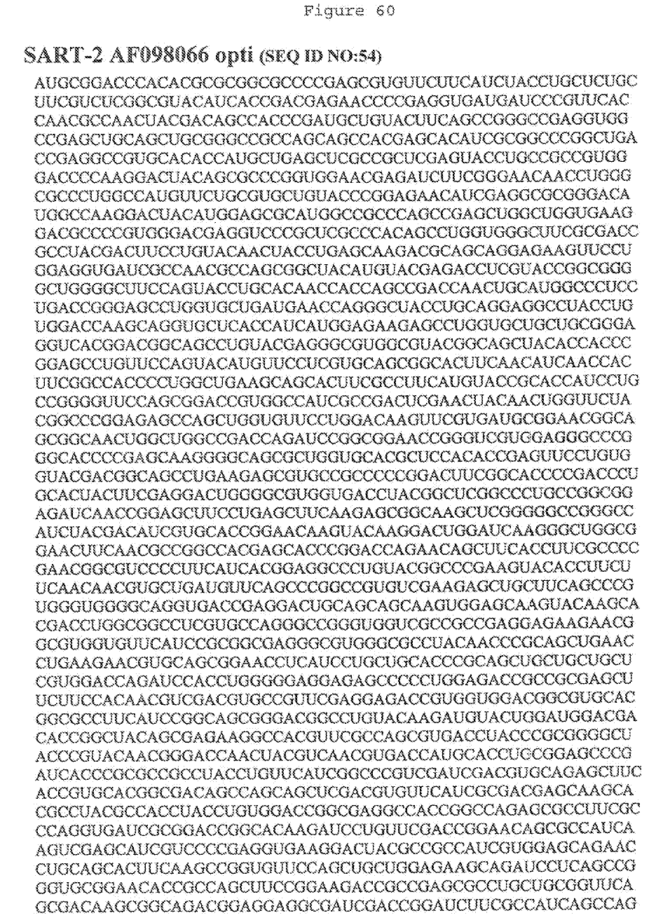

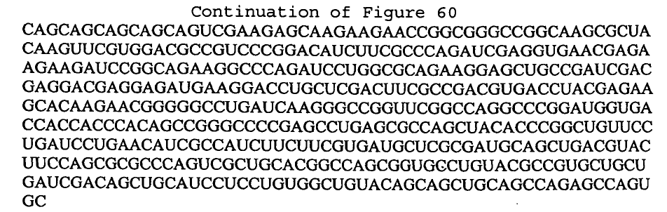

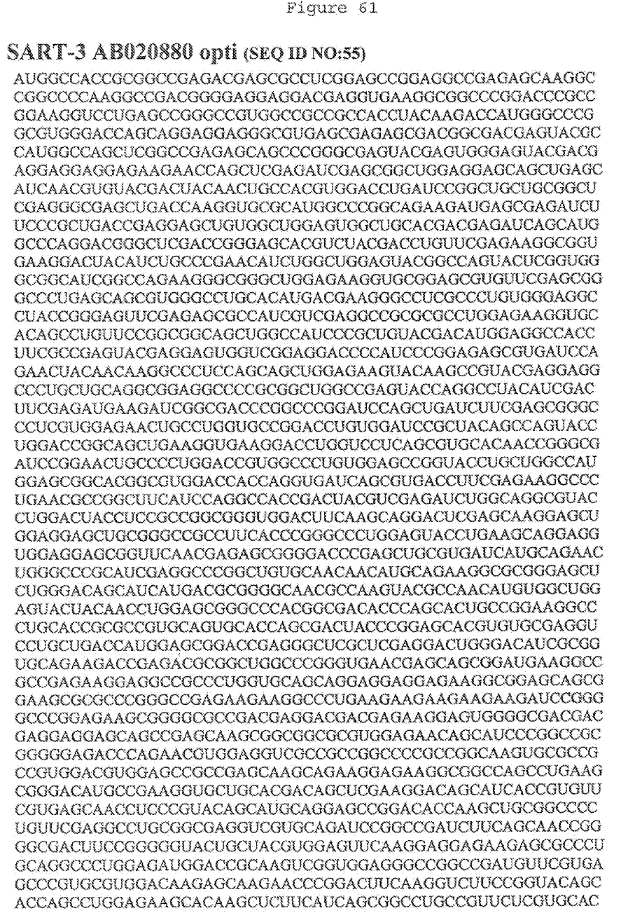

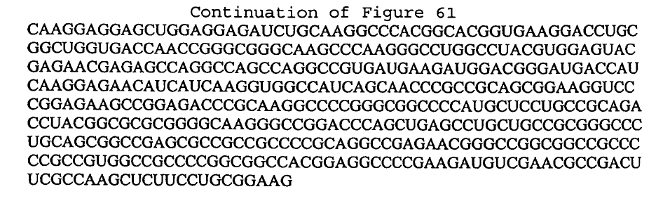

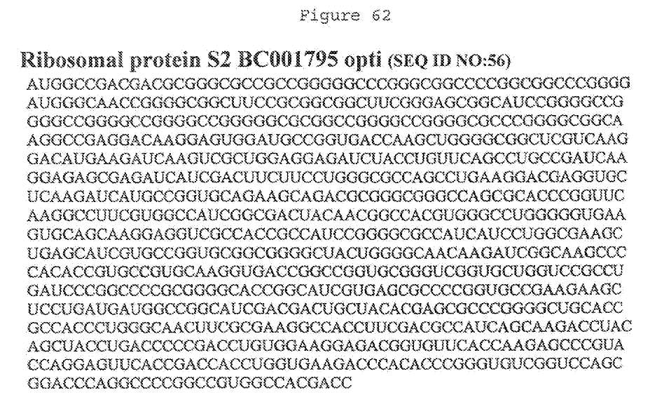

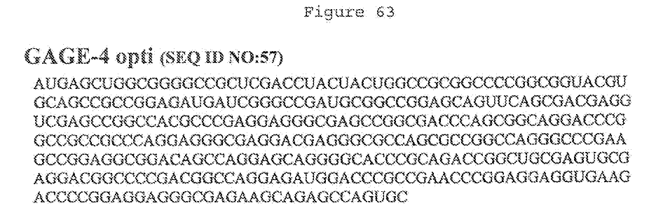

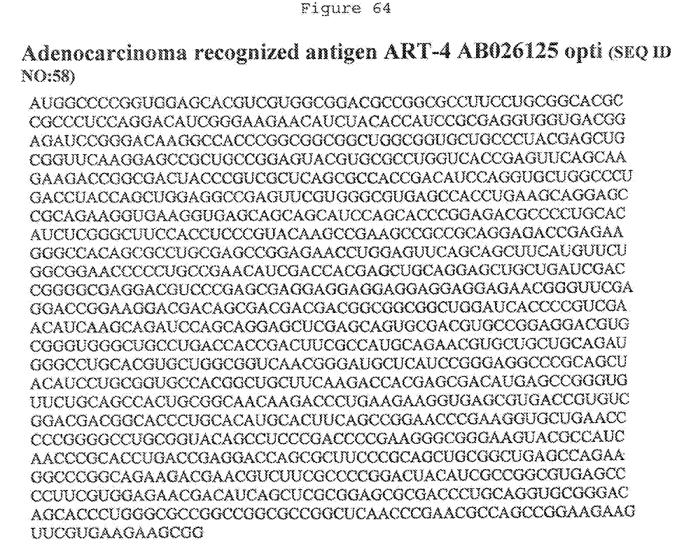

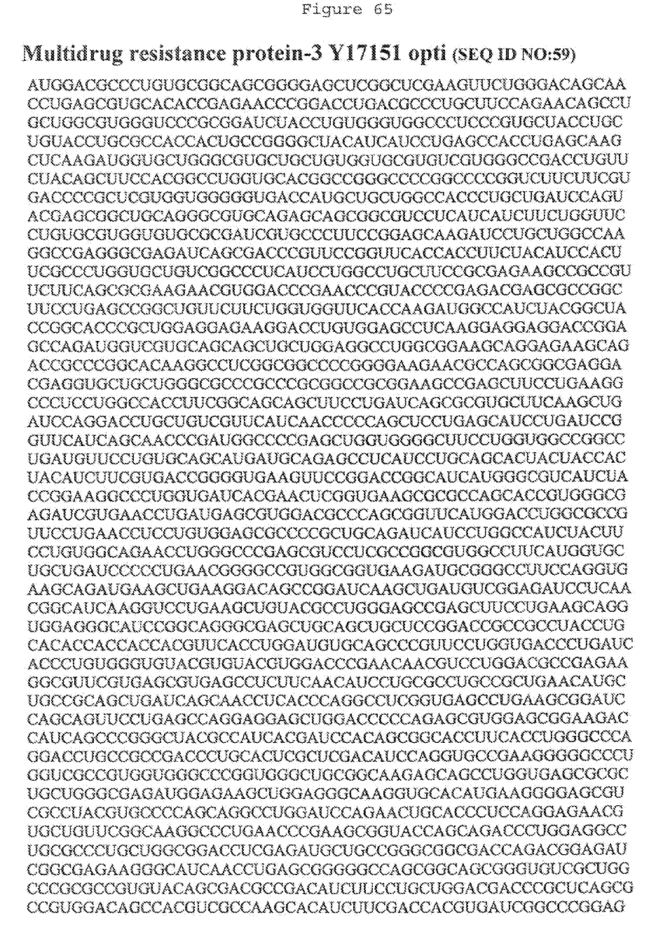

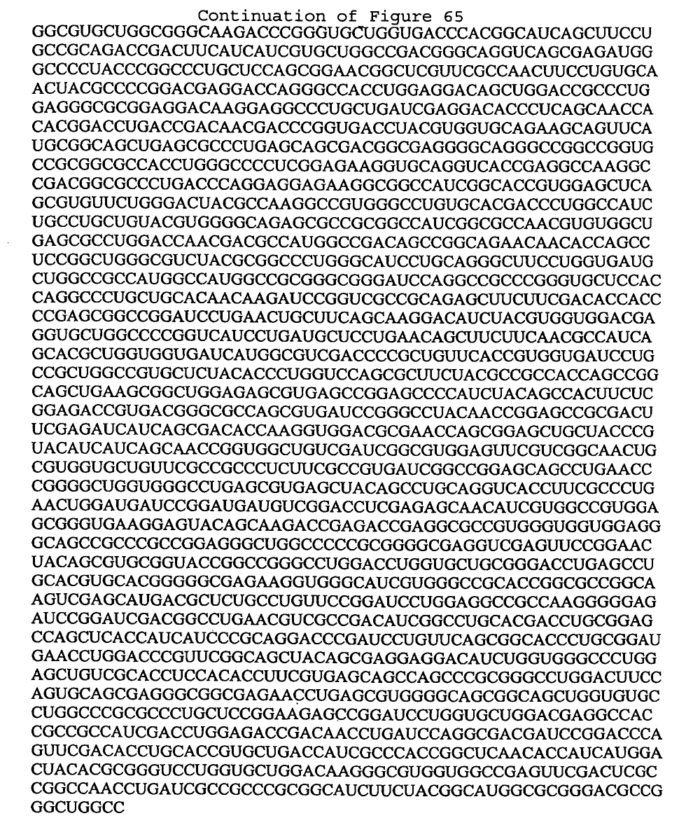

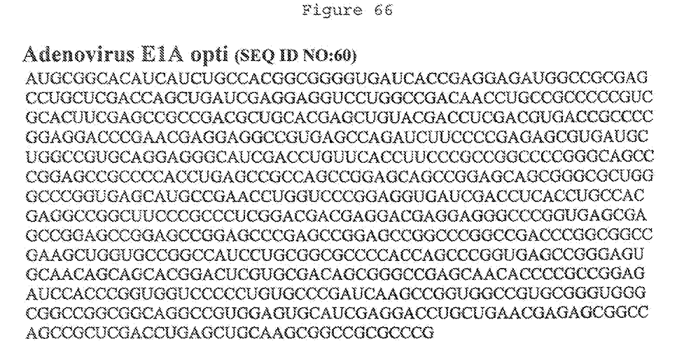

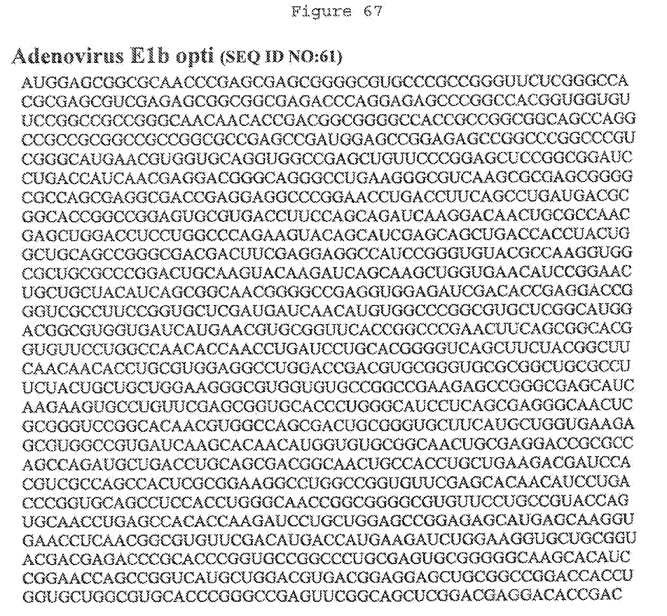

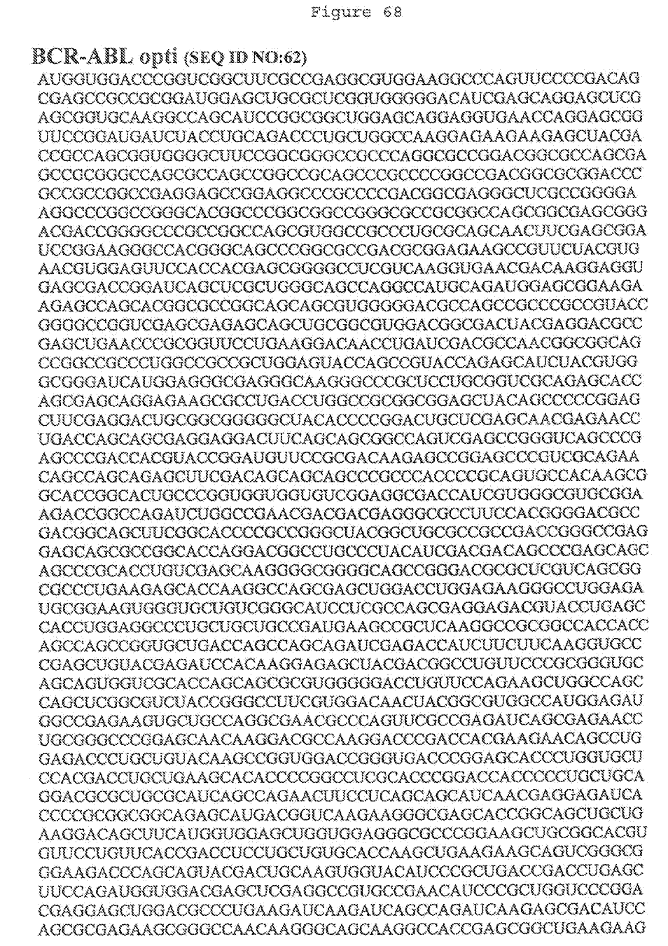

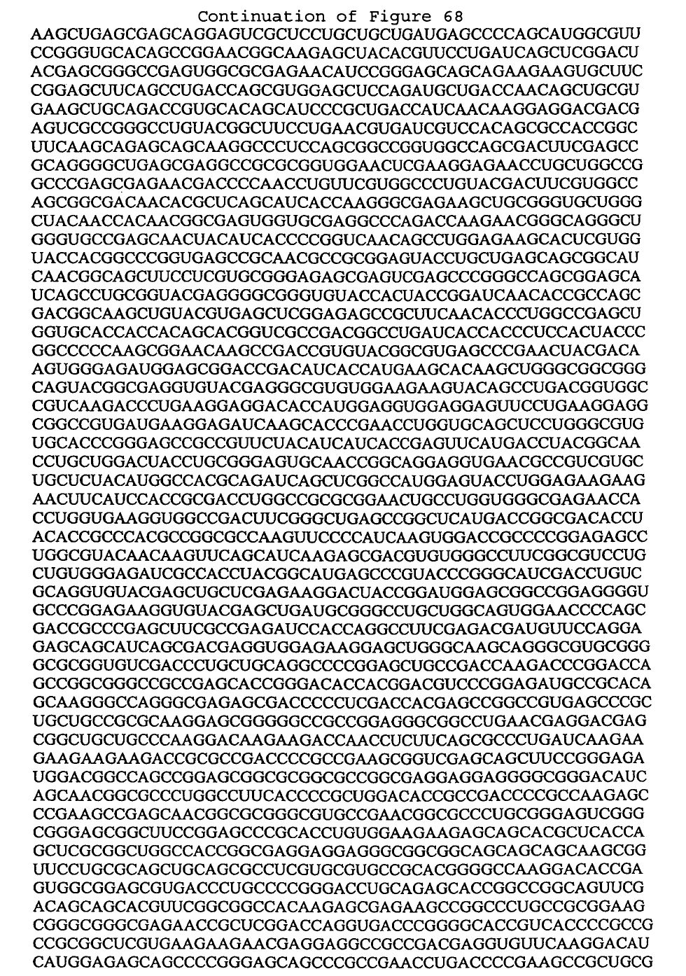

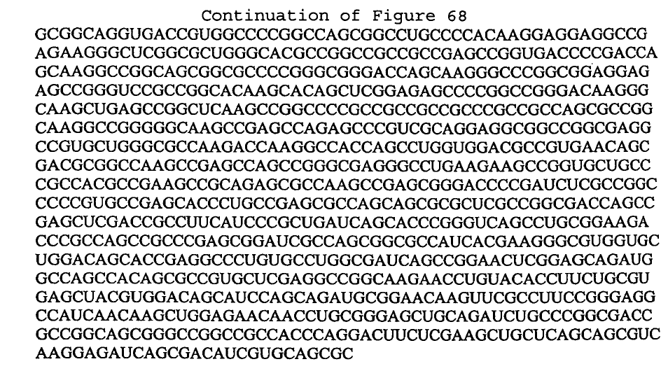

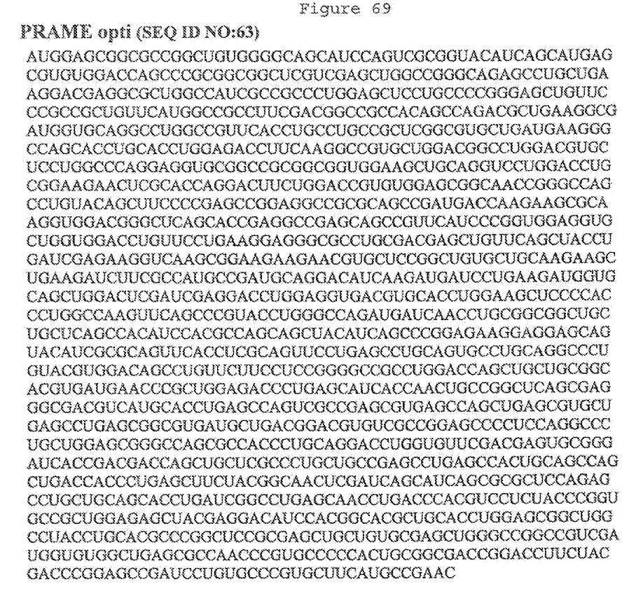

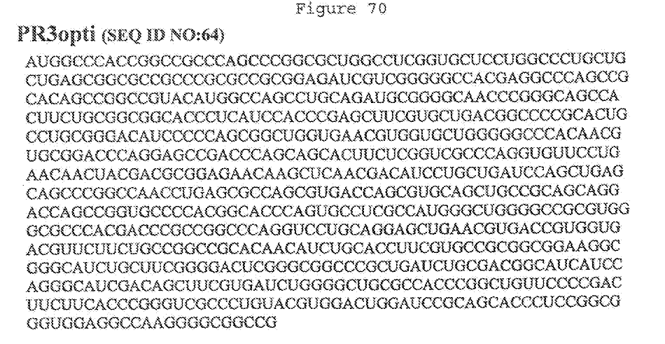

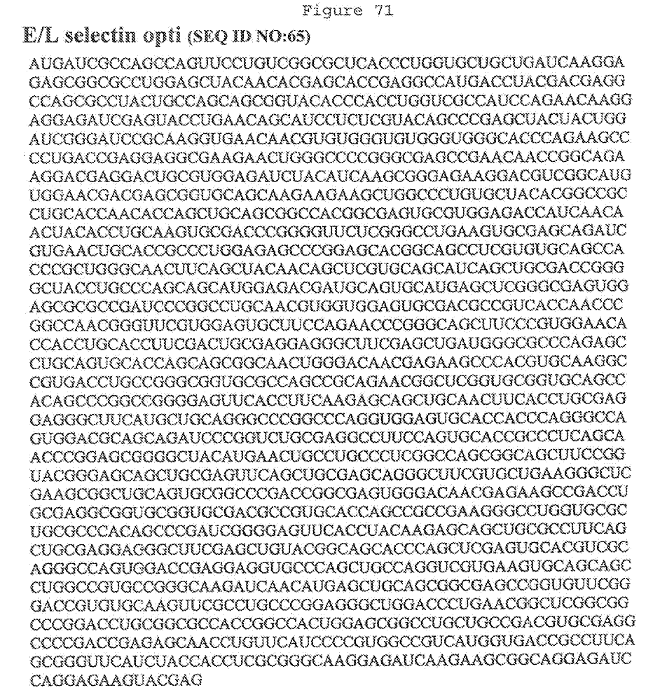

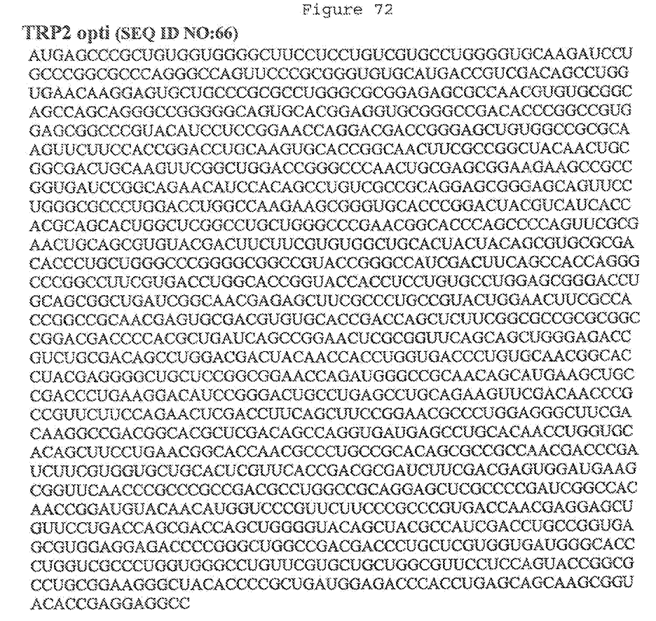

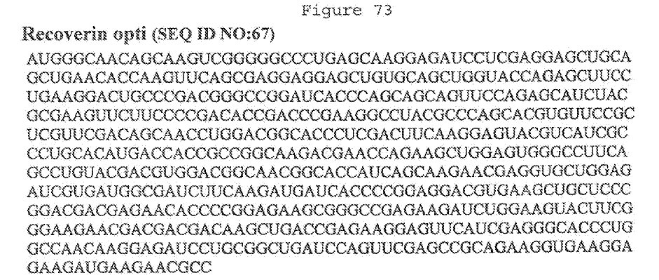

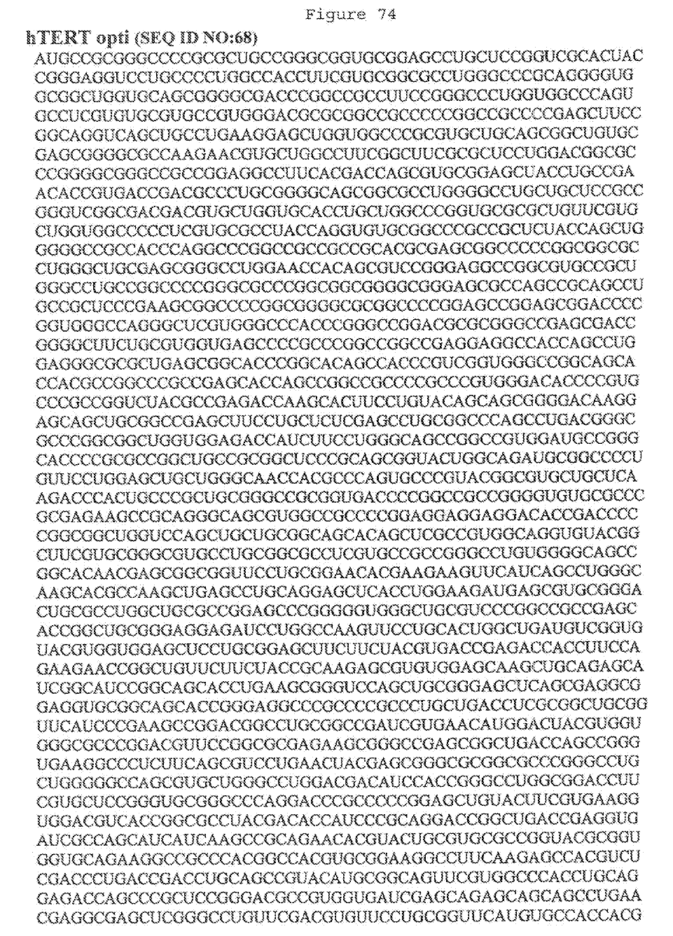

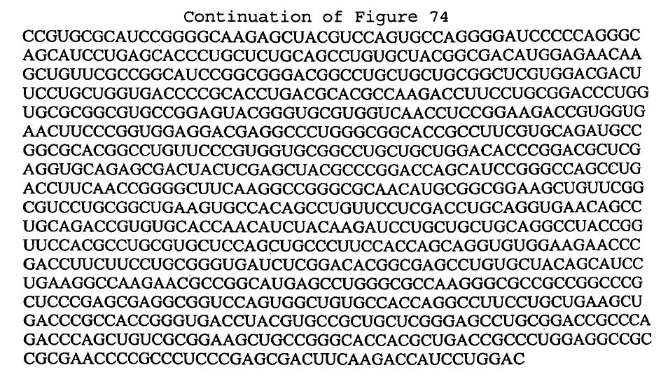

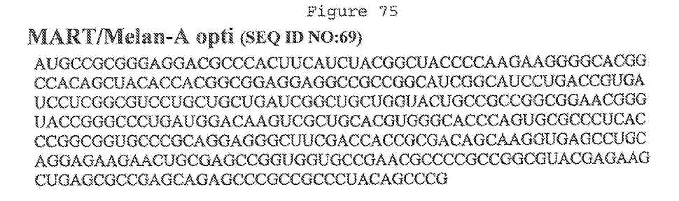

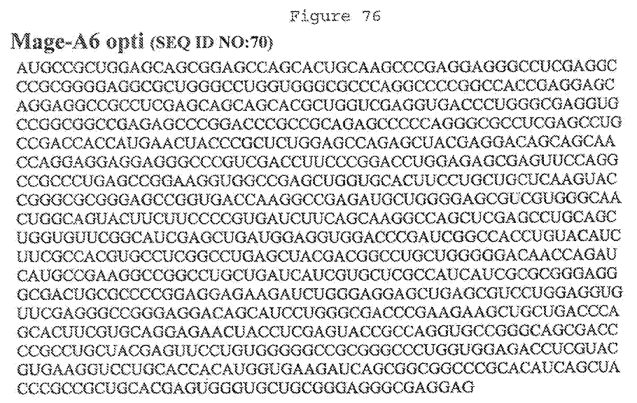

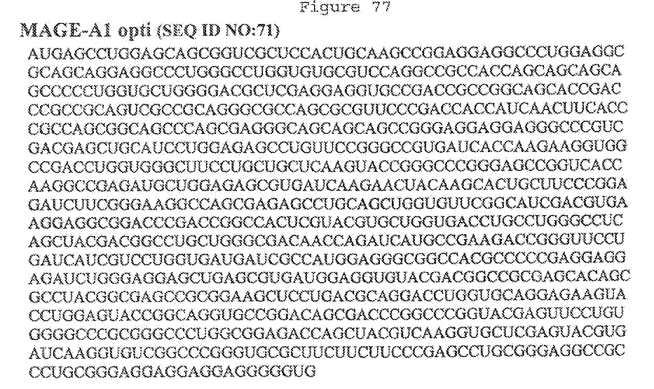

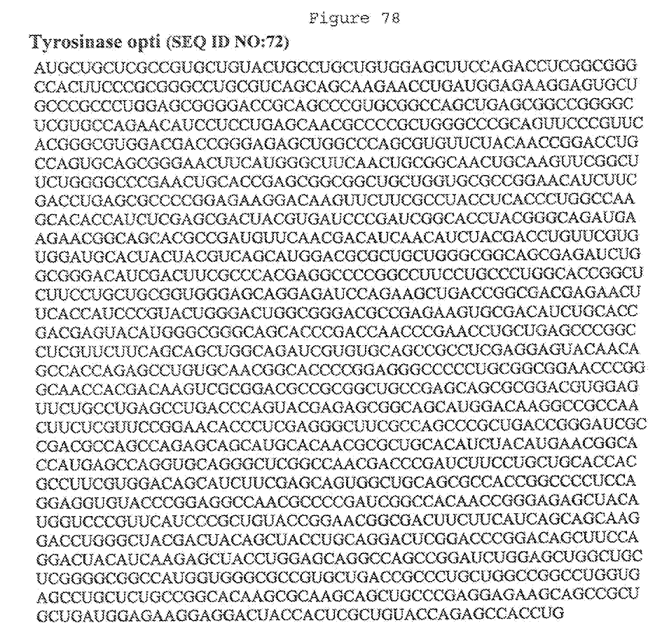

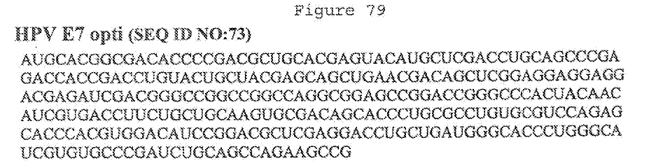

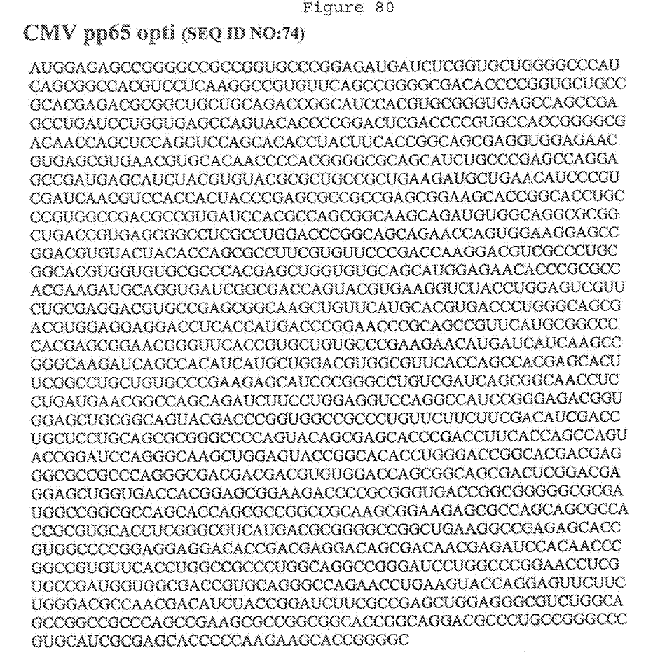

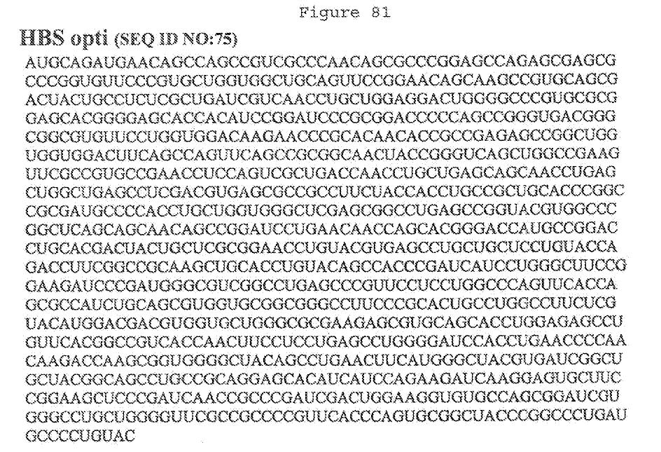

### EXAMPLES

### Example 1

### Production of the Mixture According to the Invention

The mRNA was obtained by in vitro transcription of suitable template DNA and subsequent extraction and purification of the mRNA. Standard methods, which have often been described in the prior art and are familiar to the person skilled in the art, may be used for this purpose. For example Maniatis et al. (2001), Molecular Cloning: Laboratory Manual, Cold Spring Harbor Laboratory Press. The same also applies to the mRNA sequencing which followed on from the purification (described below) of the mRNA. NBLAST software, as already described above, was in particular used here.

The mixtures according to the invention were in general produced in accordance with the following procedure:

### 1. Vector

The genes which code for the mRNAs used in the particular mixtures were introduced into plasmid vector pT7TS. pT7TS contains untranslated regions of the alpha- or beta-globin gene and a polyA-tail of 70 nucleotides:
[symbol] Xenopus β-globin 5′Untranslated region:

GCTTGTTCTTTTTGCAGAAGCTGAGAATAAACGCTCAACTTTGGC

[symbol] Xenopus β-globin 3′ untranslated region:

GACTGACTAGGATCTGGTTACCACTAAACCAGCCTCAAGAACACCCGAAT
GGAGTCTCTAAGCTACATAATACCAACTTACACTTACAAAATGTTGTCCC
CCAAAATGTAGCCATTCGTATCTGCTCCTAATAAAAAGAAAGTTTCTTCA
CATTCTA

or
human α-globin untranslated region:

CTAGTGACTGATAGCCCGCTGGGCCTCCCAACGGGCCCTCCTCCCCTCCT
TGCACC

Figure: Diagram of plasmid vector pT7TS

High purity plasmids were obtained with the Qiagen Endo-free Maxipreparation kit or with the Machery-Nagel GigaPrep kit. The sequence of the vector was checked and documented by double strand sequencing of the T7 promoter up to the PstI or XbaI site. Plasmids having an introduced cloned gene sequence which is correct and without mutations were used for the in vitro transcription.

### 2. Genes

The genes which code for the mRNAs used for mixtures according to the invention were amplified by means of PCR or extracted from the (above-described) plasmids. The following constructs were used for the “carcinoma” or “meloma” or “AML” mixtures according to the invention:

HBs (accession number E00121):
Plasmid fragment HinDIII/NsiI blunt (=with blunt end) in T7TS HinDIII/SpeI blunt
FLUWT (accession number AF348197):
Plasmid fragment SpeI blunt in T7TS BglII blunt/SpeI blunt

FLUGC-rich codes for a matrix M1 protein which [exhibits] 60%, preferably 65%, more preferably 70%, likewise more preferably 80%, likewise more preferably 85%, most preferably 90% sequence homology with the protein with the accession number V01099: plasmid fragment BglII/SpeI in T7TS BlgII/SpeI

GP100 (accession number M77348):
PCR fragment SpeI in T7TS HinDIII blunt/SpeI
MAGE-A1 (accession number M77481):
Plasmid fragment HinDIII/SpeI in T7TS HinDIII/SpeI
MAGE-A6 (accession number: NM_{—}005363):
PCR fragment SpeI in T7TS HinDIII blunt/SpeI
Her2/Neu (accession number: M11730):
PCR fragment HinDIII/SpeI in T7TS HinDIII/SpeI
Tyrosinase (accession number: NM_{—}000372):
Plasmid fragment EcoRI blunt in T7TS HinDIII blunt/SpeI blunt
Melan-A (accession number: NM_{—}005511):
Plasmid fragment NotI blunt in T7TS HinDIII blunt/SpeI blunt
CEA (accession number: NM_{—}004363):
PCR fragment HinDIII/SpeI in T7TS HinDIII/SpeI
CMV pp 65 (accession number: M15120):
PCR fragment BamHI/SpeI in T7TS BglII/SpeI
Tert (accession number: NM_{—}003219):
PCR fragment HindIII/SpeI in T7TS HinDIII/SpeI
WT1 (accession number: NM_{—}000378):
Plasmid fragment EcoRV/KpnI: blunt in T7TS HinDIII blunt/SpeI blunt
PR3 (accession number: NM_{—}002777):
Plasmid fragment EcoRI blunt/Xba1 in T7TS HinDIII blunt/SpeI
PRAME (accession number: NM_{—}006115):
Plasmid fragment BamHI blunt/XbaI in T7TS HinDIII blunt/SpeI
Survivin (accession number AF077350):
PCR fragment HinDIII/SpeI in T7TS HinDIII/SpeI
Mucin1 (accession number NM_{—}002456):
Plasmid fragment: SacI blunt/BamHI in T7TS HinDIII blunt/BglII
Tenascin (accession number X78565):
PCR fragment BglII blunt/SpeI in T7TS HinDIII blunt/SpeI
EGFR1 (accession number AF288738):
PCR fragment HinDIII/SpeI in T7TS HinDIII/SpeI
Sox9 (accession number Z46629):
PCR fragment HinDIII/SpeI in T7TS HinDIII/SpeI
Sec61G (accession number NM 014302):
PCR fragment HinDIII/SpeI in T7TS HinDIII/SpeI
PTRZ1 (accession number NM-002851):
PCR fragment EcoRV/SpeI in T7TS HinDIII blunt/SpeI

### 3. In Vitro Transcription

### 3.1. Production of Protein-Free DNA

500 μg of each of the above-described plasmids were linearized in a volume of 2.5 ml in a 15 ml Falcon tube by digestion with the restriction enzyme PstI or XbaI. This cut DNA construct was transferred into the RNA production unit. 2.5 ml of a mixture of phenol/chloroform/isoamyl alcohol was added to the linearized DNA. The reaction vessel was vortexed for 2 minutes and centrifuged for 5 minutes at 4,000 rpm. The aqueous phase was drawn off and mixed with 1.75 ml of 2-propanol in a 15 ml Falcon tube. This vessel was centrifuged for 30 minutes at 4,000 rpm, the supernatant discarded and 5 ml of 75% of ethanol were added. The reaction vessel was centrifuged for 10 minutes at 4,000 rpm and the ethanol was removed. The vessel was centrifuged for a further 2 minutes and the ethanol residues were removed with microliter pipette tip. The DNA pellet was then dissolved in 500 μl of RNase-free water (1 μg/μl).

3.2. Enzymatic mRNA Synthesis

### Materials:

T7 polymerase: purified from an E. coli strain which contains a plasmid with the gene for the polymerase. This RNA polymerase uses only T7 phage promoter sequences as substrate (from Fermentas),
NTPs: chemically synthesized and purified by HPLC. Purity greater than 96% (from Fermentas),
CAP analogue: chemically synthesized and purified by HPLC. Purity greater than 90% (Trilink),
RNase inhibitor: RNasin, injectable grade, produced by recombinant methods (E. coli) (from Fermentas),
DNase: sold as an over the counter medicine as Pulmozym® (dornase alfa) (from Roche).

The following reaction mixture is pipetted into a 15 ml Falcon tube:

100 μg of linearized protein-free DNA,
400 μl of 5× buffer (tris-HCl pH 7.5, MgCl₂, spermidine, DTT, inorganic pyrophosphatase 25 U),
20 μl of ribonuclease inhibitor (recombinant, 40 U/μl);
80 μl of rNTP mix (ATP, CTP, UTP 100 mM), 29 μl of GTP (100 mM);
116 μl of CAP analogue (100 mM);
50 μl of T7 RNA polymerase (200 U/μl);
1045 μl of RNase-free water.

Total volume was 2 ml and the mixture was incubated for 2 hours at 37° C. in a heating block. 300 μl of DNAse: Pulmozyme™ (1 U/μl) were then added and the mixture was incubated for a further 30 minutes at 37° C., so enzymatically degrading the DNA template.

5. Purification of mRNAs

### 5.1. LiCl Precipitation (Lithium Chloride/Ethanol Precipitation)

The procedure was carried out as follows relative to 20-40 μg of RNA:

### LiCl Precipitation, 25 μl of LiCl Solution [8M]

30 μl of WFI (“water for injection”) were added to the transcription batch (20 μl) and carefully mixed. 25 μl of LiCl solution were added to the reaction vessel and the solutions were vortexed for at least 10 seconds. The batch was incubated at −20° C. for at least 1 hour. The sealed vessel was then centrifuged at 4,000 rpm for 30 minutes at 4° C. The supernatant was discarded.

### Washing

5 μl of 75% ethanol were added to each pellet (in safety cabinet). The sealed vessels were centrifuged at 4,000 rpm for 20 minutes at 4° C. The supernatant was discarded (in safety cabinet) and the mixture was centrifuged at 4,000 rpm for a further 2 minutes at 4° C. The supernatant was carefully removed with a pipette (in safety cabinet). The pellet was then dried for approx. 1 hour (in safety cabinet).

### Resuspension

10 μl of WFI were added to each of the thoroughly dried pellets (in safety cabinet). Each case pellet was then dissolved overnight in a shaker at 4° C.

### 5.2. Final Purification

Final purification was carried out by phenol/chloroform extraction. It may likewise be performed by means of anion exchange chromatography (for example MEGAclear™ from Ambion or Rneasy from Qiagen). After this mRNA purification, the RNA was precipitated against isopropanol and NaCl (1 M NaCl 1:10, isopropanol 1:1, vortexed, centrifuged for 30′ at 4,000 rpm and 4° C. and the pellet was washed with 75% ethanol). The RNA purified by means of phenol/chloroform extraction was dissolved in RNase-free water and incubated for at least 12 hours at 4° C. The concentration of each mRNA was measured at OD260 absorption. (The chloroform/phenol extraction success was carried out in accordance with Sambrook J., Fritsch E. F., and Maniatis T., in Molecular Cloning: A Laboratory Manual, Cold Spring Harbor Laboratory Press, NY, vol. 1, 2, 3 (1989)).

6. Mixing of mRNAs

The purified mRNAs were mixed in the composition desired for the particular mRNA mixture according to the invention.

Identical quantities of each mRNA contained in the particular mixture according to the invention were mixed and the solution was lyophilized by freeze drying or by alcohol precipitation (isopropanol or ethanol with NaCl). The pellet was resuspended at a concentration of 5 mg/ml in RNase-free water.

This solution was subsequently diluted with buffer as required. The buffers preferably used for this purpose were:

Ringer's lactate solution (Fresenius) or PBS (phosphate buffered saline) or isotonic saline, which may also be buffered by HEPES.

It should be particularly emphasized here that the inventors achieved 5 times greater effectiveness when using Ringer's lactate solution as the buffer than when using any of the other buffers. Such values have not previously been known in the prior art. Further data in this connection may be found in Example 2 (see below).

Dilutions were prepared up to a final concentration for injection (approx. 0.6 μg/μl of RNA using a range of variation from 0.1 μg/μl up to 10 μg/μl, the concentration preferably being adjusted to 0.6 or 0.8 or 1 μg/μl). The mixture was combined as required with stabilizing cationic agents, such as for example protamine (approx. 0.12 μg/μl using a range of variation from 0.01 μg/l up to 10 μg/μl, the concentration preferably being adjusted to 0.12 or 0.16 or 0.2 μg/μl). The mixture was stored at a stable temperature of −20 to −80° C., possibly also being stored for a brief period before injection at room temperature of 4° C.

### Example 2

### Effect of Different Buffers on Expression Rate

The following experiment was performed to investigate the effectiveness of various buffers:

Identical quantities of mRNA which codes for luciferase from Photinus pyralis were injected into the ear of several mice, the mRNA being resuspended in different buffers (see Example 1) in separate test batches. The injection batches per ear contained the following compositions:

Injection Batch with Ringer's Lactate Solution
20 μl of 1 mg/ml mRNA
80 μl of 1×Ringer's lactate (#2620521, from Fresensius-Kabi)
Injection Batch with PBS
20 μl of 1 mg/ml mRNA
50 μl of 2×PBS (standard solution)
30 μl of water (H₂O)
Injection batch with HEPES/NaCl
20 μl of 1 mg/ml mRNA
50 μl of 2× buffer (20 mM Hepes, pH 7.4, 300 mM NaCl)
30 μl of water (H₂O)

The mice were killed by cervical dislocation 15 hours after the injection. The ears were removed, shaved, comminuted in nitrogen and then homogenized in lysis buffer (25 mM tris-HCl pH 7.5, 2 mM EDTA, 10% glycerol, 1% Triton X-100; further addition of DTT to 2 mM and PMSF to 1 mM). Homogenization was performed over ice. The homogenizate was then centrifuged in a microcentrifuge (Microfuge) at 4° C. for 10 min at maximum rotational speed. The supernatant was then removed, the lysate aliquoted and stored at −80° C.

Luciferase activity was detected using a standard method (“Luciferase reporter gene assay, constant light signal chemiluminescence assay for the quantitative determination of luciferase activity in transfected cells”, optimized for use with luminometers, from Roche, item no. 1 897 667). In summary, the determination (in each case carried out in duplicate) of luciferase activity was performed by measuring the light emission of 50 μl of lysate after addition of 300 μl of measurement buffer (25 mM glycyl-glycine pH 7.8, mM magnesium sulfate, 5 mM ATP (freshly added)) and 100 μl of luciferin (250 μM in water) as substrate. The measurements were made relative to an empty plate (EP) and relative to mRNA which codes for lacZ in PBS (“lacZ mRNA”) as negative controls. The result (see FIG. 13) reveals far higher expression of luciferase if the luciferase mRNA was resuspended in Ringer's lactate solution, in comparison with those injection batches in which the luciferase mRNA was resuspended in PBS or in HEPES/NaCl buffer.

### Example 3

### Stabilization of the mRNA of the Mixture According to the Invention

The nucleic acid sequence of the coding domain of the mRNAs contained in the mixture was optimized with regard to its G/C content as an example embodiment of the mixture according to the invention. The sequence of a modified mRNA according to the invention was determined using the computer software which has already been mentioned above and is described in the WO 02/098443, which, on the basis of the genetic code or the degeneracy thereof, modifies the nucleotide sequence of any desired mRNA in such a manner that a maximum G/C content is obtained in conjunction with the use of codons, which code for tRNAs which occur maximally frequently in the cell, the amino acid sequence coded by the modified mRNA preferably being identical to the unmodified sequence. Alternatively, it is also possible to modify only the G/C content or only the codon usage relative to the original sequence. The source code in Visual Basic 6.0 (development environment used: Microsoft Visual Studio Enterprise 6.0 with service pack 3) is likewise disclosed in WO 02/098443.

### Example 4

### Clinical Testing

FIG. 14 describes the performance of the clinical testing in patients treated with RNA mixtures according to the invention. The clinical results were verified by carrying out parallel investigations on blood samples which were taken from the patient before and during treatment. The blood samples were then tested for IFN-α positive cells (CD4 and CD8 cells) and the proportion of such cells in the total number of cells in each individual treated patient was determined. The increase or decrease in these cell counts correlates with the clinical phenotype of the patient and is thus a marker for therapeutic success.

Specifically, peripheral blood monocytes (PBMCs) were taken from the patient on the day of the first vaccination, on the day of the fourth vaccination (T4) etc., purified by Ficoll gradients and frozen in liquid nitrogen. At the end of the vaccination period, a tube (vial) was thawed and transfected with 10 μg of an mRNA mixture according to the invention containing identical quantities of tumor antigens (survivin+Mage-A1+Her2Neu+mucin1+CEA) or identical quantities of viral antigens (influenza GC rich+HBs). Transfection was performed by electroporation. The cells were cultured and, after one week, restimulated by newly transfected, thawed autologous peripheral blood monocytes. After a further week's culturing, some newly transfected, autologous, thawed peripheral blood monocytes were added to the culture. 6 hours later, the cells were collected, fixed and permeabilized, specifically by using the Cytofix-Cytoperm kit from BD-Pharmingen. A cocktail of antibodies was used to stain the cells: CD4-FITC, antiferon-γ PE and CD8 PercP. After washing, the cells were analyzed by FACS.

The plots according to FIGS. 16 and 17 show the number of CD4 or CD8 T lymphocytes which are reactive towards the antigen cocktail (interferon-γ positive cells). There are more reactive cells among the cells collected at the end of treatment than among the cells in the blood collected before the beginning of treatment. It may be concluded from this that the injections of the active RNA mixture has triggered an antiviral (flu and/or Hbs) and an antitumor (Her2neu and/or survivin and/or mucin-1 and/or Her2 and/or CEA) cell response. In some patients (essentially four and ten), more reactive cells were collected in the blood at the end of treatment than at the beginning of treatment. In FIG. 17, SSS is a stabilized condition after three consecutive CT scan s. P means progression.

1. A mixture which contains mRNA for vaccination, wherein at least one mRNA contains a domain which codes for at least one antigen from a tumor and at least one further mRNA contains a domain which codes for at least one immunogenic protein.

2-32. (canceled)
